SEQUENCE LISTING (93 sequences)

SEQ ID NO: 1 - DNA, 2363 bp, Homo sapiens
tgaatacgcaattagaactttcagaacaacttaaatttcagaacaactctgaagataatgttaaaaaactacaagaagagattgagaaaattaggccaggctttgaggagcaaattttatatctgcaaaagcaattagacgctaccactgatgaaaagaaggaaacagttactcaactccaaaatatcattgaggctaattctcagcattaccaaaaaaatattaatagtttgcaggaagagcttttacagttgaaagctatacaccaagaagaggtgaaagagttgatgtgccagattgaagcatcagctaaggaacatgaagcagagataaataagttgaacgagctaaaagagaacttagtaaaacaatgtgaggcaagtgaaaagaacatccagaagaaatatgaatgtgagttagaaaatttaaggaaagccacctcaaatgcaaaccaagacaatcagatatgttctattctcttgcaagaaaatacatttgtagaacaagtagtaaatgaaaaagtcaaacacttagaagataccttaaaagaacttgaatctcaacacagtatcttaaaagatgaggtaacttatatgaataatcttaagttaaaacttgaaatggatgctcaacatataaaggatgagttttttcatgaacgggaagacttagagtttaaaattaatgaattattactagctaaagaagaacagggctgtgtaattgaaaaattaaaatctgagctagcaggtttaaataaacagttttgctatactgtagaacagcataacagagaagtacagagtcttaaggaacaacatcaaaaagaaatatcagaactaaatgagacatttttgtcagattcagaaaaagaaaaattaacattaatgtttgaaatacagggtcttaaggaacagtgtgaaaacctacagcaagaaaagcaagaagcaattttaaattatgagagtttacgagagattatggaaattttacaaacagaactgggggaatctgctggaaaaataagtcaagagttcgaatcaatgaagcaacagcaagcatctgatgttcatgaactgcagcagaagctcagaactgcttttactgaaaaagatgcccttctcgaaactgtgaatcgcctccagggagaaaatgaaaagttactatctcaacaagaattggtaccagaacttgaaaataccataaagaaccttcaagaaaagaatggagtatacttacttagtctcagtcaaagagataccatgttaaaagaattagaaggaaagataaattctcttactgaggaaaaagatgattttataaataaactgaaaaattcccatgaagaaatggataatttccataagaaatgtgaaagggaagaaagattgattcttgaacttgggaagaaagtagagcaaacaatccagtacaacagtgaactagaacaaaaggtaaatgaattaacaggaggactagaggagactttaaaagaaaaggatcaaaatgaccaaaaactagaaaaacttatggttcaaatgaaagttctctctgaagacaaagaagtattgtcagctgaagtgaagtctctttatgaggaaacaataaactcagttcagaaaaaaaaacagttgagtagggatttggaggtttttttgtctcaaaaagaagatgttatccttaaagaacatattactcaattagaaaagaaacttcagttaatggttgaagagcaagataatttaaataaactgcttgaaaatgagcaagttcagaagttatttgttaaaactcagttgtatggttttcttaaagaaatgggatcagaagtttcagaagacagtgaagagaaagatgttgttaatgtcctacaggcagtcggtgaatccttggcaaaaataaatgaggaaaaatgcaacctggcttttcagcgtgatgaaaaagtattagagttagaaaaagagattaagtgccttcaagaagagagtgtagttcagtgtgaagaacttaagtctttattgagagactatgagcaagagaaagttctcttaaggaaagagttagaagaaatacagtcagaaaaagaggccctgcagtctgatcttctagaaatgaagaatgctaatgaaaaaacaaggcttgaaaatcagaatcttttaattcaagttgaagaagtatctcaaacatgtagcaaaagtgaaatccataatgaaaaagaaaaatgttttataaaggaacatgaaaacctaaagccactactagaacaaaaagaattacgagataggagagcagagttgatactattaaaggattccttagcaaaatcaccttactgtaaaaaatgatacctctgtcttcagtaa

SEQ ID NO: 2 - DNA, 3962 bp, Homo sapiens
aattcggcacgaggatgagtatagggctgtttcttgcagggttctttcgaaattattgctaggttgacttttaactaaatccaagtgatgttatttgtaatgtagtacttaaatgtttttcttgttgttttagccaaaactggacaagccaaggcatctgtagccaaagtaaacaaatctacagggaaatcagcaagttctgtaaaatctgtggtaacggtagctgttaaaggtaataaagcttcaatcaaaacagcaaaatctggtggaaagaagtctctagaagccaaaaagactgggaatgtcaaaaacaaagactctaacaaacctgtgactataccagaaaactctgaaataaagaccagtattgaagtcaaagccactgaaaactgtgctaaagaagctatttctgatgctgctttggaggccacagagaatgaaccacttaacaaggaaacagaagaaatgtgtgtgatgcttgtctctaatttgcctaataaaggatattctgtagaagaagtttatgacttagcaaaaccatttggtggtttaaaggatatcttgattttatcatctcataaaaaggcatatatagaaataaatagaaaagctgctgagtctatggtaaaattttatacctgcttcccagtattgatggatggaaatcaactctcaataagtatggctcctgaaagcatgaatataaaagatgaggaagctatatttataaccttggtaaaagaaaatgacccagaggcaaacatagatacaatttatgatcgatttgtacatcttgataatttaccggaagatggacttcagtgtgtactttgtgttggacttcagtttggaaaagtggatcaccatgtattcataagtaatagaaacaaggcaattcttcagttagatagtcctgaatctgctcagtcaatgtatagctttctgaaacaaaatccacaaaatattggtgaccatatgttgacctgctcattatctccaaagatagacttaccagaggtgcaaattgagcatgacccagaattagaaaaagaaagccctggcttgaaaaacagtccaattgatgaaagtgaggtgcaaacagcaactgatagtccctctgttaaacctaatgagcttgaagaagaaagtactcccagcattcaaacagaaactttggtacagcaggaagagccttgtgaggaagaagctgaaaaagcaacatgtgattctgactttgctgttgaaactttggagcttgaaactcaaggagaggaggtcaaagaagaaattcctcttgtagcatccgcttcagtcagtattgaacaattcactgaaaatgccgaggagtgtgctttaaatcagcagatgtttaacagtgacttggagaagaaaggggcagaaattattaaccctaaaacagcattgttaccatctgacagtgtgtttgcagaagaaaggaacctcaaaggaattctagaagaatctccatctgaagcagaagatttcatttctggaattacacagactatggtagaagctgtagctgaagtagaaaaaaatgaaactgtttcggaaatattgccatcaacttgtattgtgacgttagtaccaggaattcccactggggatgagaagacagtggacaaaaagaatatttctgaaaaaaaaggtaacatggatgaaaaggaggagaaggaatttaatactaaggaaaccagaatggatcttcaaataggaacagagaaggctgaaaagaatgaaggtaggatggatgcagaaaaggtggaaaagatggcagcaatgaaagaaaagcctgcagaaaacactttattcaaggcatacccaaataaaggagtgggtcaggctaataagcctgatgaaactagtaaaactagtattctggctgtatcagatgtatctagcagtaaaccaagcatcaaggctgttatagtctcttctcctaaggcaaaagctacagtttcaaaaactgaaaatcagaaaagttttccaaaatctgtgcccagagatcaaataaatgctgaaaagaaactttcagccaaggaatttggtctgcttaaacccacaagtgccaggtcaggcttggcagaaagcagcagtaaattcaaacctactcagagcagtcttaccagaggaggcagtggaaggatctcagccctgcaaggcaagctttctaaactggattacagagatataacaaaacaatctcaggaaacagaggctagaccttccatcatgaaacgggatgacagcaacaataagactttggctgagcaaaacactaagaatcctaaaagcactactggtagaagttccaaatctaaagaggagccattatttccatttaatttggatgaatttgttactgtggatgaggttatagaagaagtgaatccttctcaggccaagcagaatccactaaagggaaaaaggaaagaaactctcaaaaatgttcctttctctgaacttaacttaaagaagaaaaaggggaaaacttccactcctcgtggtgttgagggagaactatcttttgtgacattggatgagattggggaagaggaagatgcagctgcacatctagcacaagctctagtcactgtggatgaagtaattgatgaagaagaactaaatatggaagaaatggtaaaaaattcaaattcactttttacattagatgaattaattgaccaagatgattgcatttcccacagtgaacctaaagatgttactgttctgtcagtggctgaagaacaagatctcctcaaacaggaacgcttggtaactgtggatgaaattggagaagtggaagagctacctttgaatgagtcagcagacataacttttgccactttaaatactaaaggaaatgaaggagatatcgtaagggattccattggcttcatttcttctcaggtgcccgaagacccttctactttagttactgtagatgaaatacaagatgacagcagtgatttgcatttagtgactttggatgaagtaactgaagaggatgaagactctctggcggattttaacaaccttaaagaagagcttaattttgttactgttgatgaagttggagaggaggaagatggagataatgatttaaaagttgagttagcacaaagcaaaaatgaccatcccacagataaaaaagggaatagaaagaagagagctgtggacacaaaaaagacaaaacttgaatccttgtcccaagtgggtccagtaaatgagaatgttatggaagaagatctaaaaaccatgattgaaagacacttaacagctaaaactccaaccaagagagttagaattgggaaaactctgccatcagaaaaagctgttgtgacagaaccagcaaaaggtgaagaggccttccagatgagtgaagttgatgaggaatctggattaaaggattcagaaccagagcgaaaacgcaagaagactgaagactcttcttcaggcaaatcagtggtgtctgatgtccctgaggaattagactttcttgtacctaaggctggattcttctgtccaatttgttccctcttctactcaggtgaaaaagcaatgacaaatcactgcaagagtacacgtcataagcaaaatactgagaaattcatggccaagcaaagaaaggaaaaggagcagaatgaggctgaagaaagaagctctaggtgattgggggaaaggaaagaattcactagaaatttgtttagggtccagttgatttgtgtatttttgttatcatttaatttgtaattttcgtttcagaagcaaatattcgtgttgtacaaatttctgattgccctaaatgtagagagactgatggggaaagtatgatgggtttgatttttatatcaaatcatcaggcatggagaaatatcttttagaagtgttaaaataaatgttcctactgtatatttaaaataaaaaaaaaaaaaaaaaaa

SEQ ID NO: 3 - DNA, 2710 bp, Homo sapiens
ggagatacaagtttggaagcaatcttggggtacttacccacaaggctggtggagaccagatcaggagaacctcagtctgacgacattgaagctagccgaatgaagcgagcagctgcaaagcatctaatagaacgctactaccaccagttaactgagggctgtggaaatgaagcctgcacgaatgagttttgtgcttcctgtccaacttttcttcgtatggataataatgcagcagctattaaagccctcgagctttataagattaatgcaaaactctgtgatcctcatccctccaagaaaggagcaagctcagcttaccttgagaactcgaaaggtgcccccaacaactcctgctctgagataaaaatgaacaagaaaggcgctagaattgattttaaagatgtgacttacttaacagaagagaaggtatatgaaattcttgaattatgtagagaaagagaggattattcccctttaatccgtgttattggaagagttttttctagtgctgaggcattggtacagagcttccggaaagttaaacaacacaccaaggaagaactgaaatctcttcaagcaaaagatgaagacaaagatgaagatgaaaaggaaaaagctgcatgttctgctgctgctatggaagaagactcagaagcatcttcctcaaggataggtgatagctcacagggagacaacaatttgcaaaaattaggccctgatgatgtgtctgtggatattgatgccattagaagggtctacaccagattgctctctaatgaaaaaattgaaactgcctttctcaatgcacttgtatatttgtcacctaacgtggaatgtgacttgacgtatcacaatgtatactctcgagatcctaattatctgaatttgttcattatcgtaatggagaatagaaatctccacagtcctgaatatctggaaatggctttgccattattttgcaaagcgatgagcaagctaccccttgcagcccaaggaaaactgatcagactgtggtctaaatacaatgcagaccagattcggagaatgatggagacatttcagcaacttattacttataaagtcataagcaatgaatttaacagtcgaaatctagtgaatgatgatgatgccattgttgctgcttcgaagtgcttgaaaatggtttactatgcaaatgtagtgggaggggaagtggacacaaatcacaatgaagaagatgatgaagagcccatccctgagtccagcgagctgacacttcaggaacttttgggagaagaaagaagaaacaagaaaggtcctcgagtggaccccctggaaactgaacttggtgttaaaaccctggattgtcgaaaaccacttatcccttttgaagagtttattaatgaaccactgaatgaggttctagaaatggataaagattatacttttttcaaagtagaaacagagaacaaattctcttttatgacatgtccctttatattgaatgctgtcacaaagaatttgggattatattatgacaatagaattcgcatgtacagtgaacgaagaatcactgttctctacagcttagttcaaggacagcagttgaatccatatttgagactcaaagttagacgtgaccatatcatagatgatgcacttgtccggctagagatgatcgctatggaaaatcctgcagacttgaagaagcagttgtatgtggaatttgaaggagaacaaggagttgatgagggaggtgtttccaaagaattttttcagctggttgtggaggaaatcttcaatccagatattggtatgttcacatacgatgaatctacaaaattgttttggtttaatccatcttcttttgaaactgagggtcagtttactctgattggcatagtactgggtctggctatttacaataactgtatactggatgtacattttcccatggttgtctacaggaagctaatggggaaaaaaggaacttttcgtgacttgggagactctcacccagttctatatcagagtttaaaagatttattggagtatgaagggaatgtggaagatgacatgatgatcactttccagatatcacagacagatctttttggtaacccaatgatgtatgatctaaaggaaaatggtgataaaattccaattacaaatgaaaacaggaaggaatttgtcaatctttattctgactacattctcaataaatcagtagaaaaacagttcaaggcttttcggagaggttttcatatggtgaccaatgaatctcccttaaagtacttattcagaccagaagaaattgaattgcttatatgtggaagccggaatctagatttccaagcactagaagaaactacagaatatgacggtggctataccagggactctgttctgattagggagttctgggaaatcgttcattcatttacagatgaacagaaaagactcttcttgcagtttacaacgggcacagacagagcacctgtgggaggactaggaaaattaaagatgattatagccaaaaatggcccagacacagaaaggttacctacatctcatacttgctttaatgtgcttttacttccggaatactcaagcaaagaaaaacttaaagagagattgttgaaggccatcacgtatgccaaaggatttggcatgctgtaaaacaaaacaaaacaaaataaaacaaaaaaaaaaaaaaaaaaaaaaaaaaaaa

SEQ ID NO: 4 - DNA, 3997 bp, Homo sapiens
ctggccccaaggtccgatcgcccaggggaggagcagcaccgggaccccgcgtcggctgggcgccccacaagggaagccagtcttaatatgatggaaacatctctgaacttctaaaagaccaaggttggcgttttagctctattaattttacttcgtcttggccagaattcacaatgacaacagtgacagtgaccacagaaattcccccaagggataagatggaagataattctgccttgtatgagtctacgtccgctcacattattgaagaaaccgagtatgtgaaaaagattcgaactactctgcaaaagatcaggacccagatgtttaaagatgaaataagacatgacagtacaaatcacaaactagatgcaaagcactgtggaaaccttcaacagggctctgattctgaaatggatccttcttgttgcagtttggatttgcttatgaaaaagataaaaggaaaagacctacagctcttagaaatgaacaaagagaatgaagtattgaaaatcaagctgcaagcctccagagaagcaggagcagcagctctgagaaacgtggcccagagattatttgaaaactaccaaacgcaatctgaagaagtgagaaagaagcaggaggacagtaaacaattactccaggttaacaagcttgaaaaagaacagaaattgaaacaacatgttgaaaatctgaatcaagttgctgaaaaacttgaagaaaaacacagtcaaattacagaattggagaaccttgtacagagaatggaaaaggaaaagagaacactactagaaagaaaactgtctttggaaaacaagctactgcaactcaaatccagtgctacatatggaaaaagttgccaggatcttcagagggagatttccattctccaggagcagatctctcatctgcagtttgtgattcactcccaacatcagaacctgcgcagtgtcatccaggagatggaaggattaaaaaataatttaaaagaacaagacaaaagaattgaaaatctcagagaaaaggttaacatacttgaagcccagaataaagaactaaaaacccaggtagcactttcatctgaaactcctaggacaaaggtatctaaggctgtctctacaagtgaattgaagaccgaaggtgtttccccttatttaatgttgattaggttacggaaatgaactggctggatgaagatctgatttagaaagactgcgtgagtcttatttattctctgaaacacagcccaagtttcatgttaaaatggcaaaatgccattatttaaatggaacttattacataccaatggctttgcaagaagatgacatttcagaagatcaaacaaatctatatttaatggatggactcttcaaaacttaccaaatagttgaagaaaccaggtgccttctcatgatggaagacagattctgctttaaattaaaaaaaaaaaaaatctgaatcttgttttcagatttttttttctactgggattgttttaagattgtcaattctgacttttttatagtggtttttaagagtataaatagaagggagagtgtatatgtgtatgaatgaacatacatttcctgcatatatatgtatgaagggcatgtatatgtatgaatgagcaaacatatttgaaagttaacttttggatgataggaaagatcgtacagtgacataagttcatctcctgatccattgtttgtgggagaattatacttgactgaattatgggcaggagaaagagcagattccttttagctaattccccaacccatatgcccctctgaagttgagaatcatggctgcctcaccacacatcagagaatgactgttcttcttagttctgggattaaaaattggtttctagaggtaacctgtacacacaaacgcacagggatgcacatggtttcctctgcctttgtgactaattttcttcttgatagttattaatagtatctaaataaaatattggggggatagaaaaataatgctgttagctcatacttccatgaaaatgtatatattataggctcaaaggaataatgactgctgtctgcagccagaaagaatctgaatttatgaattggaaagatatatatagtgtatttgtgaaagtttgcttaaattctgatacatgccttctttgtaggtggagtttgtgattgcagtgaatagaacaaattctgacttgcagaaatgcagacatacagtcaggaaagaacaaacttcaattaaatgtataatgagagaccttggtccccctaaaggatgaatttcctttaggcctttgatcttcctctctagtgtataactttaaatatttgctcagaaaagatgctgactctttcattatggaatgtgaaatatcagtgttgtctataaatatttgaaggtatataaaaatgagatgatgtaatgtatttataaatttatccaagtactgtaatccttgaattgttgtgtaactgtgtgtgagttttatgcttcatggtatttttggaaacatttttattgcttgcttattttgaaggtatatttatctgttaacatttagggcatagttaccttataccagcaacaagtctaagcactttacttgtattaatttattttatcttccccaaggccctcagaagcagattattcctgttttacagagaaagatactgagagtggttgagtaatttgcccagctgctaactgtgaagcaaaaatttgaaccttttgacttggctattgatattcattctacttgctcacatggtggtctaagaaatttcccagctatagaaatctctctatttttgccactttaatcaacacatagcttcctggatgactgcctgtgttattttgtggatgacagtaagaaacaacaaatactgataaaatcaatattttgctgaaatgagttgatctttcaccagctggactaccattgtgagaactcagttcagacaaacttccctgctaaaaatctgtttatcatacatttattatttatgactttatgtcacattgaagaatttcttcatgatacattttcaggcacacttgtaggaaaattaggatcatgagtcctgctttaagtatttgcagtgtagtaagagaatccatcttttactaggagaccagattccttttatacctcattcatcatgctggattgtaataaatttcagattttggaatgggcttatttaactgacctaacaatcttgatgatttccattagaataacttattctaaggtcaaaagtggaaagacactgttggtttttattttgatttcactatactcatttttgaacatggaaatacagtggtgaaaccacctatgcaaaaatgataacagtgaggaaattatgacagtgaaagagatctgacctaactatctatcttgcctcgaaactgcccttggtcgttcctgagtgtgggccaagctaactttgggagaaatttactttataggttaaattataatagcccttcccaaaactaaacgattctcctgcctcagcctcccgagtagctgtctttataataccatcagccttatcatttattcgtcatgtatggattgtttcctatatccactatcataaaattatcatttgaaatatttttttatgaaaaaaaaaacacttctcagtgaaaaaacaagattacaagggggaataatatttattcagctaaaatagtacttcacaggaaatataggaaaagaaacactagttcagttttattccaaataatgtacttctaattatactcttgaatttattggataagaaggtctgaggtgggtctgaaattcatacatgaagtcaggaaaagaaaagaaacttagttctaccttgattacatatgttgttacaaattatcattataaaatgtttaaacaattagtatagtatatcttttgaataattgcttataatatgccttaccataaagaaaattgatgctaaaaaaaaaaaaaaaaaaaaaa

SEQ ID NO: 5 - DNA, 1592 bp, Homo sapiens
ctgctttgaaggctgcacagaaaaccttacttgtttccacctctgcagttgataataatgaagcacagaaaaaaaaacaggaggcattgaaacttcagcaggatgtaaggaaaaggaaacaagaaattttagaaaagcacattgaaacacagaagatgttaatttcaaaactggagaaaaacaaaacaatgaagtctgaagataaagcagaaataatgaaaactttagaggttttgacaaaaaatattaccaagttgaaagatgaggtcaaagctgcttctcctggacgctgtcttccaaaaagtataaaaaccaagactcagatgcagaaggaattacttgacacagaactggatttatataagaagatgcaggctggagaagaagtcactgaacttaggagaaagtatacagaattacagctggaagctgccaaacgagggattctttcatctggtcggggcagaggaattcattcaagaggtcgaggtgcagttcatggccgaggcagggggcgagggcgagggcgaggtgtgcctggtcatgctgtggtggatcaccgtcccagggcattggagatttctgcatttacggagagcgatagagaagatcttcttcctcattttgcgcaatatggtgaaattgaagattgtcagattgatgattcctcacttcatgcagtaattacattcaagacaagagcagaagctgaagcagctgcagttcatggagctcgtttcaaagggcaagatctaaaactggcatggaataaaccagtaactaatatttcagctgttgaaacagaagaagttgagcctgatgaagaagaatttcaggaagagtctttggtggatgactcattacttcaagatgatgatgaagaagaagaggacaatgaatctcgttcttggagaagatgatttgactgatcattgatctgcatatgctagaactctacctgtgtttcattagtattatctaatgtacttttacatatttgtaaaaacaatttttggtaaaatgtgatgaagatggatttcacaaatagacaaaaaagaagaaaactaccttctgatcttgtattttgaaagattgatgtttgcattttacttcagtaaacaattgctaaagacatcacactagaaacatatgcaatgtttttattacatacttctactggacatcacagaattctttgggttctttgtaatttaatgaataggtctgaaaacttatgaccaatacttgttataacttagaggactttgttttattccaaataaggaatgaatttgcatttaaaatcttaatgaatgttttcaaaactgaatagataacatagtactctaactaaagtctccaagttatgtattataatattacatagtagtatgcttaggctttactatgtattagccttttgttggactgtgtatgtattttaccatatgggttttaatgataatggtgtatgactgctttacatgagtccttatgcatccagatgttataataaagtggaatggtctctttaaaaaaaaaaaaggaaagaaaagagaaaagcaatgacaaaaaaaaaaaaaaaaaaaaaa

SEQ ID NO: 6 - DNA, 2171 bp, Homo sapiens
aattcggcacgaggattcttgtgccaaaacagacataggctcagaaaattctgaacaaatagctaattttcctagtggaaattttgctaaacatatttcaaaaacaaatgaaacagaacagaaagtaacacaaatattggtggaattaaggtcatctacatttccagaatcagctaatgaaaagacttattcagaaagcccctatgatacagactgcaccaagaaatttatttcaaaaataaagagcgtttcagcatcagaggatttgttggaagaaatagaatctgagctcttatctacggagtttgcagaacatcaagtaccaaatggaatgaataagggagaacatgcattagttctgtttgaaaagtgtgtgcaagataaatatttgcagcaggaacatatcataaaaaagttaattaaagaaaataagaagcatcaggagctcttcgtagacatttgttcagaaaaagacaatttaagagaagaactaaagaaaagaacagaaactgagaagcagcatatgaacacaattaaacagttagaatcaagaatagaagaacttaataaagaagttaaagcttccagagataaactaatagctcaagacgttacagctaaaaatgcagttcagcagttacacaaagagatggcccaacggatggaacaggccaacaagaaatgtgaagaggcacgccaagaaaaagaagcaatggtaatgaaatatgtaagaggtgagaaggaatctttagatcttcgaaaggaaaaagagacacttgagaaaaaacttagagatgcaaataaggaacttgagaaaaacactaacaaaattaagcagctttctcaggagaaaggacggttgcaccagctgtatgaaactaaggaaggcgaaacgactagactcatcagagaaatagacaaattaaaggaagacattaactctcacgtcatcaaagtaaagtgggcacaaaacaaattaaaagctgaaatggattcacacaaggaaaccaaagataaactcaaagaaacaacaacaaaattaacacaagcaaaggaagaagcagatcagatacgaaaaaactgtcaggatatgataaaaacatatcaggagtcagaagaaattaaatcaaatgagcttgatgcaaagcttagagtcacaaaaggagaacttgaaaaacaaatgcaagaaaaatctgaccagctagagatgcatcatgccaaaataaaggaactagaagatctgaagagaacatttaaggagggtatggatgagttaagaacactgagaacaaaggtgaaatgtctagaagatgaacgattaagaacagaagatgaattatcaaaatataaggaaattattaatcgccaaaaagctgaaattcagaatttattggacaaggtgaaaactgcagatcagctacaggagcagcttcaaagaggtaagcaagaaattgaaaatttgaaagaagaagtggaaagtcttaattctttgattaatgacctacaaaaagacatcgaaggcagtaggaaaagagaatctgagctgctgctgtttacagaaaggctcactagtaagaatgcacagcttcagtctgaatccaattctttgcagtcacaatttgataaagtttcctgtagtgaaagtcagttacaaagccagtgtgaacaaatgaaacagacaaatattaatttggaaagtaggttgttgaaagaggaagaactgcgaaaagaggaagtccaaactctgcaagctgaactcgcttgtagacaaacagaagttaaagcattgagtacccaggtagaagaattaaaagatgagttagtaactcagagacgtaaacatgcctctagtatcaaggatctcaccaaacaacttcagcaagcacgaagaaaattagatcaggttgagagtggaagctatgacaaagaagtcagcagcatgggaagtcgttctagttcatcagggtccctgaatgctcgaagcagtgcagaagatcgatctccagaaaatactgggtcctcagtagctgtggataactttccacaagtagataaggccatgttgattgagagaatagttaggctgcaaaaagcacatgcccggaaaaatgaaaagatagaatttatggaggaccacatcaaacaactggtggaaaaaaaaaaaaaaaaaa

SEQ ID NO: 7 - DNA, 3513 bp, Homo sapiens
ctcgaaattaaccctcactaaagggaacaaaagctggagctccaccgcggtggcggccgctctagaactagtggatcccccgggctgcaggaattcggcacgaggtgatgctgctgctgctgctgctgctgccgccgccgcctctattgctgatactctagtggggctggaagggtggttcctattcgcaccatcgccaaccagagacagagggaaaaaaaaaaccggcagccactgctgatgttgggttcggaggctgcatccgactcggtcacaaggaaaatggattcagtttgcatctctccctcctttaaacagcttctccgggtctcagcatgggcttccagggcagcgattgaggagaccttaccaaggagcaccacacagtagatgctgagacatcgtactccaggataagaaacagtaacatggcagcacctgcttgaaagaaattaaaaaccaacagactccatttagaaaggaacaatgtccaagaaagggcgaaataagggcgagaagcccgaggcactcattgttgcccttcaagctgccaatgaagacctcaggaccaagctcacagacattcagatagagctgcatcaagagaagtccaaggtatcaaagcttgaaagagagaagactcaagaagcgaagaggattcgtgagctggagcagcgcaagcacacggtgctggtgacagaactcaaagccaagctccatgaggagaagatgaaggagctgcaggctgtgagggagaaccttatcaagcagcacgagcaggaaatgtcaaggacggtgaaggtacgtgatggagagatccagaggctcaagtctgctctctgtgctctccgcgacggcagcagtgacaaagtaaggacagcgctcaccattgaggcccgggaggaggcccggaaactgtttgacacagagcgccttaagctcttacaggaaattgcggacctgaaaacggccaagaagcaggtggacgaggctctgagcaatatgatccaagcagataaaatcaaggctggggaccttcggagtgagcatcagtcccaccaagaagccatctcgaagatcaagtgggagtcggagcgggatattcggaggctgatggatgaaatcaaagccaaggacaggatcatcttttccctggaaaaggaactggagacccagacaggctatgtacagaaactccaacttcagaaggaggctttggacgaacaactctttctggtcaaggaggctgagtgcaacatgagcagcccaaaacgagaaattccaggaagggcaggtgatggttccgaacactgcagcagtcctgatttgcgaagaaatcaaaagagaatagctgaattgaatgccactataagaaaattagaagacaggaataccttgcttggagatgaacgaaatgaactgttaaaacgtgtgcgggaaaccgaaaagcaatgtaaacctctcctggaaaggaacaagtgcctcgccaagagaaacgatgaactgatggtgtccttgcagcgcatggaagaaaaactaaaagccgttaccaaggaaaattcagaaatgagagaaaaaataacatcccatccacccctgaagaaattaaaatctctgaatgacctcgaccaagctaatgaagaacaagaaacagagtttctaaaacttcaggtcattgagcaacagaacattattgatgagctcacaagggaccgagaaaagctcatccgtagaagaaagcatagaagaagttccaagccaattaagaggcctgttttggacccgtttattggctatgatgaggactctatggattcagagacatcatccatggcctcatttagaacagacagaacaccagctactcctgatgatgacttggatgaaagtttagcagctgaagaatctgaactaagatttcgacaattaacaaaagaatatcaggccctccaaagagcatatgccctcctacaggagcagacgggaggcatcatcgacgctgaacgagaagccaaggctcaagaacagctccaagcagaggtgctaaggtataaagccaaaattgaagacctggaagcgactctggctcagaaagggcaggattcacactgggtagaagataaacaacttttcattaagagaaaccaggagcttttagaaaagatagaaaaacaggaggcagaaaatcaccggttacaacaagaactacaggacgccagagaccagaatgagctgctggagtttcgaaacctagagctagaagagagagagagacgatcccctccatttaatctccaaattcacccattctcagatggtgtgagtgctctacagatctactgtatgaaagaaggtgttaaggatgtgaacatccctgatctcataaagcagcttgatatcttgggtgataatgggaatttaagaaatgaagaacaagtggccataattcaggccagcactgtgctgtccctggcagagaagtggatccagcagattgaaggagctgaggctgccctacaccagaaaatgatggaattggaaagtgacatggaacagttctgcaaaataaaaggctatctggaggaagaactagactacagaaaacaagctcttgaccaagcatatatgagaatccaggaactagaagctactttgtacaatgctctacagcaagaaactgttatcaagtttggtgaattattaagtgaaaaacagcaagaggagctgaggacggcagtagaaaagttacggcggcaaatgctgaggaagagcagagagtatgactgtcagattcttcaggagagaatggagctcttacagcaagcccatcagagaattcgtgacttagaagataaaacagacatccagaaaagacaaataaaagacttagaagaaaagagtaaccgaaaacatggataagatcccaggaagacaagtgcttctaaaccttcaaagatggcaaaattgtttacaccagtgagagggagatcaaaagctaagaactaccctgtagccaggactacaactgtgtattttaaagccattattcaaggtttcttacttgacagttcctacacaaccctgttgaaaatctacaatatatgctgcatttaatgaaacatgtatatgtcaaatcagaagagaagaactataaacatatattgtgtaaagaaaaagttcagcaatggaactagtttctgcagatcaagcaaagatgtgtcttgggcatggaaccaaagttacaatgaaatattcaacccctgctgtgcaggggggtcattttaatgtaacaccacaccccatggaaacactagtcctgataataaacatcattttaaaagatcaaaacaaacaaacaaaaaaaacaagggtgggtggggagtgaagcacgaggaatacctatgaagagctatttacaataaaatgtttcatttgaaaagtcaaaaaaaaaaaaaaaaaaactcgagggggggcccggtacccaattcgccctatagtgagtcgtattacaat

SEQ ID NO: 8 - DNA, 1465 bp, Homo sapiens
aattcggcacgaggcgcgggccgctgtgaggcgcggcggcgagcgacgggcgcggggccgcggagcagcgagcgagcgagcgagcgcgaggccggagccccggccaggcccggccgacccgccgagcccgcgatgcgccccggggccgccccccggcgcagctgacgccccgcggccccgcgaagaccccggccggccggtcccggaggaagcggccgccgccgccgccgcccagcccagcgcccgcgccgcccgggcaccatggcggggaaggcggccgccccgggcaccgcggtgctgctggtcacggccaacgtgggctcgctcttcgacgacccagaaaacctgcagaagaactggcttcgggaattttaccaggtcgtgcacacacacaggccgcacttcatggccttgcactgtcaggagtttggagggaagaactacgaggcctccatgtcccacgtggacaagttcgtcaaagaactattgtcgagtgatgcgatgaaagaatataacagggctcgagtctacctggatgaaaactacaaatcccaggagcacttcacggcactaggaagcttttattttcttcatgagtccttaaaaaacatctaccagtttgactttaaagctaagaagtatagaaaggtcgctggcaaagagatctactcggataccttagagagcacgcccatgctggagaaggagaagtttccgcaggactacttccccgagtgcaaatggtcaagaaaaggcttcatccggacgaggtggtgcattgcagactgtgcctttgacttggtgaatatccatcttttccatgatgcttccaatctggtcgcctgggaaacaagcccttccgtgtactcgggaatccggcacaaggcactgggctacgtgctggacagaatcattgatcagcgattcgagaaggtttcctactttgtatttggtgatttcaacttccggctggattccaagtccgtcgtggagacgctctgcacaaaagccaccatgcagacggtccgggccgccgacaccaatgaagtggtgaagctcatatttcgtgagtcggacaacgaccggaaggttatgctccagttagaaaagaaactcttcgactacttcaaccaggaggttttccgagacaacaacggcaccgcgctcttggagtttgacaaggagttgtctgtctttaaggacagactgtatgaactggacatctcgttccctcccagctacccgtacagtgaggacgcccgccagggtgagcagtacatgaacacccggtgcccagcctggtgtgaccgcatcctcatgtccccgtctgccaaggagctggtgctgcgggtgagtgtgtgctgccccagccctgggcacagagggatgtggagcgctgggtctggtctggcccagccctggtgacagggccccaggggtgggggaaaaaaaaaaaaaaaaaaaa

SEQ ID NO: 9 - DNA, 1681 bp, Homo sapiens
gagggttagatcgagcaaccctctaaaagcagtttagagtggtaaaaaaaaaaaaaacacaccaaacgctcgcagccacaaaagggatgaaatttcttctggacatcctcctgcttctcccgttactgatcgtctgctccctagagtccttcgtgaagctttttattcctaagaggagaaaatcagtcaccggcgaaatcgtgctgattacaggagctgggcatggaattgggagactgactgcctatgaatttgctaaacttaaaagcaagctggttctctgggatataaataagcatggactggaggaaacagctgccaaatgcaagggactgggtgccaaggttcatacctttgtggtagactgcagcaaccgagaagatatttacagctctgcaaagaaggtgaaggcagaaattggagatgttagtattttagtaaataatgctggtgtagtctatacatcagatttgtttgctacacaagatcctcagattgaaaagacttttgaagttaatgtacttgcacatttctggactacaaaggcatttcttcctgcaatgacgaagaataaccatggccatattgtcactgtggcttcggcagctggacatgtttcggtccccttcttactggcttactgttcaagcaagtttgctgctgttggatttcataaaactttgacagatgaactggctgccttacaaataactggagtcaaaacaacatgtctgtgtcctaatttcgtaaacactggcttcatcaaaaatccaagtacaagtttgggacccactctggaacctgaggaagtggtaaacaggctgatgcatgggattctgactgagcagaagatgatttttattccatcttctatagcttttttaacaacattggaaaggatccttcctgagcgtttcctggcagttttaaaacgaaaaatcagtgttaagtttgatgcagttattggatataaaatgaaagcgcaataagcacctagttttctgaaaactgatttaccaggtttaggttgatgtcatctaatagtgccagaattttaatgtttgaacttctgttttttctaattatccccatttcttcaatatcatttttgaggctttggcagtcttcatttactaccacttgttctttagccaaaagctgattacatatgatataaacagagaaatacctttagaggtgactttaaggaaaatgaagaaaaagaaccaaaatgactttattaaaataatttccaagattatttgtggctcacctgaaggctttgcaaaatttgtaccataaccgtttatttaacatatatttttatttttgattgcacttaaattttgtataatttgtgtttctttttctgttctacataaaatcagaaacttcaagctctctaaataaaatgaaggactatatctagtggtatttcacaatgaatatcatgaactctcaatgggtaggtttcatcctacccattgccactctgtttcctgagagatacctcacattccaatgccaaacatttctgcacagggaagctagaggtggatacacgtgttgcaagtataaaagcatcactgggatttaaggagaattgagagaatgtacccacaaatggcagcaataataaatggatcacacttaaaaaaaaaaaaaaaaaaaaaaaaaaaaa

SEQ ID NO: 10 - DNA, 1980 bp, Homo sapiens
aattcggcacgaggttcactctttgcaataaatcttgctgctgctcactctttgggtccacactgcctttatgagctgtaacactcactgggaatgtctgcagcttcactcctgaagccagcgagaccacgaacccaccaggaggaacaaacaactccagacgcgcagccttaagagctgtaacactcaccgcgaaggtctgcagcttcactcctgagccagccagaccacgaacccaccagaaggaagaaactccaaacacatccgaacatcagaaggagcaaactcctgacacgccacctttaagaaccgtgacactcaacgctagggtccgcggcttcattcttgaagtcagtgagaccaagaacccaccaattccggacacgctaattgttgtagatcatcacttcaaggtgcccatatctttctagtggaaaaattattctggcctccgctgcatacaaatcaggcaaccagaattctacatatataaggcaaagtaacatcctagacatggctttagagatccacatgtcagaccccatgtgcctcatcgagaactttaatgagcagctgaaggttaatcaggaagctttggagatcctgtctgccattacgcaacctgtagttgtggtagcgattgtgggcctctatcgcactggcaaatcctacctgatgaacaagctggctgggaagaacaagggcttctctgtgcatctacggtgcagtctcacaccaagggaatttggatatggtgtgtgcctcatcccaactggccaaatcacacattagttctgcttgacaccgagggcctgggagatgtagagaaggctgacaacaagaatgatatccagatctttgcactggcactcttagtgagcagcacctttgtgtacaatactgtgaacaaaattgatcagggtgctatcgacctactgcacaatgtgacagaactgacagatctgctcaaggcaagaaactcacccgaccttgacagggttgaagatcctgctgactctgcgagcttcttcccagacttagtgtggactctgagagatttctgcttaggcctggaaatagatgggcaacttgtcacaccagatgaatacctggagaattccctaaggccaaagcaaggtagtgatcaaagagttcaaaatttcaatttgccccgtctgtgtatacagaagttctttccaaaaaagaaatgctttatctttgacttacctgctcaccaaaaaaagcttgcccaacttgaaacactgcctgatgatgagctagagcctgaatttgtgcaacaagtgacagaattctgttcctacatctttagccattctatgaccaagactcttccaggtggcatcatggtcaatggatctcgtctaaagaacctggtgctgacctatgtcaatgccatcagcagtggggatctgccttgcatagagaatgcagtcctggccttggctcagagagagaactcagctgcagtgcaaaaggccattgcccactatgaccagcaaatgggccagaaagtgcagctgcccatggaaaccctccaggagctgctggacctgcacaggaccagtgagagggaggccattgaagtcttcatgaaaaactctttcaaggatgtagaccaaagtttccagaaagaattggagactctactagatgcaaaacagaatgacatttgtaaacggaacctggaagcatcctcggattattgctcggctttacttaaggatatttttggtcctctagaagaagcagtgaagcagggaatttattctaagccaggaggccataatctcttcattcagaaaacagaagaactgaaggcaaagtactatcgggagcctcggaaaggaatacaggctgaagaagttctgcagaaatatttaaagtccaaggagtctgtgagtcatgcaatattacagactgaccaggctctcacagagacggaaaaaaaaaaaaaaaaaa

SEQ ID NO: 11 - DNA, 1494 bp, Homo sapiens
aattcggcacgagggtggaaatttgagaccagcaagtactatgtgactatcattgatgccccaggacacagagactttatcaaaaacatgattacagggacatctcaggctgactgtgctgtcctgattgttgctgctggtgttggtgaatttgaagctggtatctccaagaatgggcagacccgagagcatgcccttctggcttacacactgggtgtgaaacaactaattgtcggtgttaacaaaatggattccactgagccaccctacagccagaagagatatgaggaaattgttaaggaagtcagcacttacattaagaaaattggctacaaccccgacacagtagcatttgtgccaatttctggttggaatggtgacaacatgctggagccaagtgctaacatgccttggttcaagggatggaaagtcacccgtaaggatggcaatgccagtggaaccacgctgcttgaggctctggactgcatcctaccaccaactcgtccaactgacaagcccttgcgcctgcctctccaggatgtctacaaaattggtggtattggtactgttcctgttggccgagtggagactggtgttctcaaacccggtatggtggtcacctttgctccagtcaacgttacaacggaagtaaaatctgtcgaaatgcaccatgaagctttgagtgaagctcttcctggggacaatgtgggcttcaatgtcaagaatgtgtctgtcaaggatgttcgtcgtggcaacgttgctggtgacagcaaaaatgacccaccaatggaagcagctggcttcactgctcaggtgattatcctgaaccatccaggccaaataagcgccggctatgcccctgtattggattgccacacggctcacattgcatgcaagtttgctgagctgaaggaaaagattgatcgccgttctggtaaaaagctggaagatggccctaaattcttgaagtctggtgatgctgccattgttgatatggttcctggcaagcccatgtgtgttgagagcttctcagactatccacctttgggtcgctttgctgttcgtgatatgagacagacagttgcggtgggtgtcatcaaagcagtggacaagaaggctgctggagctggcaaggtcaccaagtctgcccagaaagctcagaaggctaaatgaatattatccctaatacctgccaccccactcttaatcagtggtggaagaacggtctcagaactgtttgtttcaattggccatttaagtttagtagtaaaagactggttaatgataacaatgcatcgtaaaaccttcagaaggaaaggagaatgttttgtggaccactttggttttcttttttgcgtgtggcagttttaagttattagtttttaaaatcagtactttttaatggaaacaacttgaccaaaaatttgtcacagaattttgagacccattaaaaaagttaaatgagaaaaaaaaaaaaaaaaaaaaaaaaaaaaa

SEQ ID NO: 12 - DNA, 2564 bp, Homo sapiens
ccacgcgctccgggccgctcaggctgagcgatttcccgccttttctgaggttctgaggcgggagccattggttctttctgttgccctcatagaccgtatgtagcagttcgcgtgggcacagaacccacggtttcccgctagttcttcaaagtagatatttacaaccgtaacagagaaaatggaaaagcaaaagccctttgcattgttcgtaccaccgagatcaagcagcagtcaggtgtctgcggtgaaacctcagaccctgggaggcgattccactttcttcaagagtttcaacaaatgtactgaagatgattttgagtttccatttgcaaagactaatctctccaaaaatggggaaaacattgattcagatcctgctttacaaaaagttaatttcttgcccgtgcttgagcaggttggtaattctgactgtcactatcaggaaggactaaaagactctgatttggagaattcagagggattgagcagagtgtattcaaaactgtataaggaggctgaaaagataaaaaaatggaaagtaagtacagaagctgaactgagacagaaagaaagtaagttgcaagaaaacagaaagataattgaagcacagcgaaaagccattcaggaactgcaatttggaaatgaaaaagtaagtttgaaattagaagaaggaatacaagaaaataaagatttaataaaagagaataatgccacaaggcatttatgtaatctactcaaagaaacctgtgctagatctgcagaaaagacaaagaaatatgaatatgaacgggaagaaaccaggcaagtttatatggatctaaataataacattgagaaaatgataacagcttttgaggaacttcgtgtgcaagctgagaattccagactggaaatgcattttaagttaaaggaagattatgaaaaaatccaacaccttgaacaagaatacaagaaggaaataaatgacaaggaaaagcaggtatcactactattgatccaaatcactgagaaagaaaataaaatgaaagatttaacatttctgctagaggaatccagagataaagttaatcaattagaggaaaagacaaaattacagagtgaaaacttaaaacaatcaattgagaaacagcatcatttgactaaagaactagaagatattaaagtgtcattacaaagaagtgtgagtactcaaaaggctttagaggaagatttacagatagcaacaaaaacaatttgtcagctaactgaagaaaaagaaactcaaatggaagaatctaataaagctagagctgctcattcgtttgtggttactgaatttgaaactactgtctgcagcttggaagaattattgagaacagaacagcaaagattggaaaaaaatgaagatcaattgaaaatacttaccatggagcttcaaaagaaatcaagtgagctggaagagatgactaagcttacaaataacaaagaagtagaacttgaagaattgaaaaaagtcttgggagaaaaggaaacacttttatatgaaaataaacaatttgagaagattgctgaagaattaaaaggaacagaacaagaactaattggtcttctccaagccagagagaaagaagtacatgatttggaaatacagttaactgccattaccacaagtgaacagtattattcaaaagaggttaaagatctaaaaactgagcttgaaaacgagaagcttaagaatactgaattaacttcacactgcaacaagctttcactagaaaacaaagagctcacacaggaaacaagtgatatgaccctagaactcaagaatcagcaagaagatattaataataacaaaaagcaagaagaaaggatgttgaaacaaatagaaaatcttcaagaaacagaaacccaattaagaaatgaactagaatatgtgagagaagagctaaaacagaaaagagatgaagttaaatgtaaattggacaagagtgaagaaaattgtaacaatttaaggaaacaagttgaaaataaaaacaagtatattgaagaacttcagcaggagaataaggccttgaaaaaaaaaggtacagcagaaagcaagcaactgaatgtttatgagataaaggtcaataaattagagttagaactagaaagtgccaaacagaaatttggagaaatcacagacacctatcagaaagaaattgaggacaaaaagatatcagaagaaaatcttttggaagaggttgagaaagcaaaagtaatagctgatgaagcagtaaaattacagaaagaaattgataagcgatgtcaacataaaatagctgaaatggtagcacttatggaaaaacataagcaccaatatgataagatcattgaagaaagagactcagaattaggactttataagagcaaagaacaagaacagtcatcactgagagcatctttggagattgaactatccaatctcaaagctgaacttttgtctgttaagaagcaacttgaaatagaaagagaagagaaggaaaaactcaaaagagaggcaaaagaaaacacagctactcttaaagaaaaaaaaaaaaaaaaaa

SEQ ID NO: 13 - DNA, 2317 bp, Homo sapiens
ctaccaacaagcattttattcgtctggctgagatggaacagacagtagcagaacaagatgactctctttcctcactcttggtcaaactaaagaaagtatcacaagatttggagagacaaagagaaatcactgaattaaaagtaaaagaatttgaaaatatcaaattacagcttcaagaaaaccatgaagatgaagtgaaaaaagtaaaagcggaagtagaggatttaaagtatcttctggaccagtcacaaaaggagtcacagtgtttaaaatctgaacttcaggctcaaaaagaagcaaattcaagagctccaacaactacaatgagaaatctagtagaacggctaaagagccaattagccttgaaggagaaacaacagaaagcacttagtcgggcacttttagaactccgggcagaaatgacagcagctgctgaagaacgtattatttctgcaacttctcaaaaagaggcccatctcaatgttcaacaaatcgttgatcgacatactagagagctaaagacacaagttgaagatttaaatgaaaatcttttaaaattgaaagaagcacttaaaacaagtaaaaacagagaaaactcactaactgataatttgaatgacttaaataatgaactgcaaaagaaacaaaaagcctataataaaatacttagagagaaagaggaaattgatcaagagaatgatgaactgaaaaggcaaattaaaagactaaccagtggattacagggcaaacccctgacagataataaacaaagtctaattgaagaactccaaaggaaagttaaaaaactagagaaccaattagagggaaaggtggaggaagtagacctaaaacctatgaaagaaaagaatgctaaagaagaattaattaggtgggaagaaggtaaaaagtggcaagccaaaatagaaggaattcgaaacaagttaaaagagaaagagggggaagtctttactttaacaaagcagttgaatactttgaaggatctttttgccaaagccgataaagagaaacttactttgcagaggaaactaaaaacaactggcatgactgttgatcaggttttgggaatacgagctttggagtcagaaaaagaattggaagaattaaaaaagagaaatcttgacttagaaaatgatatattgtatatgagggcccaccaagctcttcctcgagattctgttgtagaagatttacatttacaaaatagatacctccaagaaaaacttcatgctttagaaaaacagttttcaaaggatacatattctaagccttcaatttcaggaatagagtcagatgatcattgtcagagagaacaggagcttcagaaggaaaacttgaagttgtcatctgaaaatattgaactgaaatttcagcttgaacaagcaaataaagatttgccaagattaaagaatcaagtcagagatttgaaggaaatgtgtgaatttcttaagaaagaaaaagcagaagttcagcggaaacttggccatgttagagggtctggtagaagtggaaagacaatcccagaactggaaaaaaccattggtttaatgaaaaaagtagttgaaaaagtccagagagaaaatgaacagttgaaaaaagcatcaggaatattgactagtgaaaaaatggctaatattgagcaggaaaatgaaaaattgaaggctgaattagaaaaacttaaagctcatcttgggcatcagttgagcatgcactatgaatccaagaccaaaggcacagaaaaaattattgctgaaaatgaaaggcttcgtaaagaacttaaaaaagaaactgatgctgcagagaaattacggatagcaaagaataatttagagatattaaatgagaagatgacagttcaactagaagagactggtaagagattgcagtttgcagaaagcagaggtccacagcttgaaggtgctgacagtaagagctggaaatccattgtggttacaagaatgtatgaaaccaagttaaaagaattggaaactgatattgccaaaaaaaatcaaagcattactgaccttaaacagcttgtaaaagaagcaacagagagagaacaaaaagttaacaaatacaatgaagaccttgaacaacagattaagattcttaaacatgttcctgaaggtgctgagacagagcaaggccttaaacgggagcttcaagttcttagattagctaatcatcagctggataaagagaaagcagaattaatccatcagatagaagctaacaaggaccaaagtggagctgaaagcaccatacctgatgctgatcaactaaaaaaaaaaaaaaaaaa

SEQ ID NO: 14 - DNA, 2234 bp, Homo sapiens
aggatgaggatgggacagaagaggataacagtcgtgttgaacctgttggacatgctgacacgggtttggagcatatacccaacttttctctggatgatatggtaaagctcgtagaagtccccaacgatggagggcctctgggaatccatgtagtgcctttcagtgctcgaggcggcagaaccctggggttattagtaaaacgattggagaaaggtggtaaagctgaacatctgaacatgaaaatctttttcgtgagaatgattgcattgtcaggattaatgatggcgaccttcgaaatagaagatttgaacaagcacaacatatgtttcgccaagccatgcgtacacccatcatttggttccatgtggttcctgcagcaaataaagagcagtatgaacaactatcccaaagtgagaagaacaattactattcaagccgttttagccctgacagccagtatattgacaacaggagtgtgaacagtgcagggcttcacacggtgcagagagcaccccgactgaaccacccgcctgagcagatagactctcactcaagactacctcatagcgcacacccctcgggaaaaccaccatccgctccagcctcggcacctcagaatgtatttagtacgactgtaagcagtggttataacaccaaaaaaataggcaagaggcttaatatccagcttaagaaaggtacagaaggtttggaattcagcatcacttccagagatgtaacaataggtggctcagctccaatctatgtgaaaaacattctcccccggggggcggccattcaggatggccgacttaaggcaggagacagacttatagaggtaaatggagtagatttagtgggcaaatcccaagaggaagttgtttcgctgttgagaagcaccaagatggaaggaactgtgagccttctggtctttcgccaggaagacgccttccacccaagggaactgaatgcagagccaagccagatgcagattccaaaagaaacgaaagcagaagatgaggatattgttcttacacctgatggcaccagggaatttctgacatttgaagtcccacttaatgattcaggatctgcaggccttggtgtcagtgtcaaaggtaaccggtcaaaagagaaccacgcagatttgggaatctttgtcaagtccattattaatggaggagcagcatctaaagatggaaggcttcgggtgaatgatcaactgatagcagtaaatggagaatccctgttgggcaagacaaaccaagatgccatggaaaccctaagaaggtctatgtctactgaaggcaataaacgaggaatgatccagcttattgttgcaaggagaataagcaagtgcaatgagctgaagtcacctgggagcccccctggacctgagctgcccattgaaacagcgttggatgatagagaacgaagaatttcccattccctctacagtgggattgaggggcttgatgaatcgcccagcagaaatgctgccctcagtaggataatgggtgagtcaggtaaataccagctgtcccctacagtgaatatgccccaagatgacactgtcattatagaagatgacaggttgccagtgcttcctccacatctctctgaccagtcctcttccagctcccatgatgatgtggggtttgtgacggcagatgctggtacttgggccaaggctgcaatcagtgattcagccgactgctctttgagtccagatgttgatccagttcttgcttttcaacgagaaggatttggacgtcagatagctgacgagactaaactcaatacagtggatgaccagaaagcaggttctcccagcagagatgtgggtccttccctgggtctgaagaagtcaagctcgttggagagtctgcagaccgcagttgccgaggtgactttgaatggggatattcctttccatcgtccacggccgcggataatcagaggcaggggatgcaatgagagcttcagagctgccatcgacaaatcttatgataaacccgcggtagatgatgatgatgaaggcatggagaccttggaagaagacacagaagaaagttcaagatcagggagagagtctgtatccacagccagtgatcagccttcccactctctggagagacaaatgaatggaaaccaagagaaaggtgataagactgatagaaaaaaaaaaaaaaaaaaaaaaaaaaaaaaaaaaaaaaaaaaaaaaaaaaaaaaaaaaa

SEQ ID NO: 15 - DNA, 2620 bp, Homo sapiens
atcaaatgctgctcgatcccaccaaccccagcgccggcactgccaagatagacaagcaggagaaggtcaagctcaactttgacatgacggcatcccccaagatcctgatgagcaagcctgtgctgagtgggggcacaggccgccggatttccttgtcggatatgccgcgctcccccatgagcacaaactcttctgtgcacacgggctccgacgtggagcaggatgctgagaagaaggccacgtcgagccacttcagtgcgagcgaggagtccatggacttcctggataagagcacagcttcaccagcctccaccaagacgggacaagcagggagtttatccggcagcccaaagcccttctctcctcaactgtcagctcctatcacgacgaaaacggacaaaacctccaccaccggcagcatcctgaatcttaacctggatcgaagcaaagctgagatggatttgaaggagctgagcgagtcggtccagcaacagtccacccctgttcctctcatctctcccaagcgccagattcgtagcaggttccagctgaatcttgacaagaccatagagagttgcaaagcacaattaggcataaatgaaatctcggaagatgtctatacggccgtagagcacagcgattcggaggattctgagaagtcagatagtagcgatagtgagtatatcagtgatgatgagcagaagtctaagaacgagccagaagacacagaggacaaagaaggttgtcagatggacaaagagccatctgctgttaaaaaaaagcccaagcctacaaacccagtggagattaaagaggagctgaaaagcacgtcaccagccagcgagaaggcagaccctggagcagtcaaggacaaggccagccctgagcctgagaaggacttttccgaaaaggcaaaaccttcacctcaccccataaaggataaactgaagggaaaagatgagacggattccccaacagtccatttgggcctggactctgattcagagagcgaacttgtcatagatttaggagaagaccattctgggcgggagggtcgaaaaaataagaaggaacccaaagaaccatctcccaaacaggatgttgtaggtaaaactccaccatccacgacggtgggcagccattctcccccggaaacaccggtgctcacccgctcttccgcccaaacttccgcggctggcgccacagccaccaccagcacgtcctccacggtcaccgtcacggccccggcccccgccgccacaggaagcccagtgaaaaagcagaggccgcttttaccgaaggagactgccccggccgtgcagcgggtcgtgtggaactcatcaactgtccagcagaaggagatcacacagagcccatccacgtccaccatcaccctggtgaccagcacacagtcatcggccctggtcaccagctcggggtccatgagcacccttgtgtcctcagtcaacgctgacctgcccatcgccactgcctcagctgatgtcgccgctgatattgccaagtacactagcaaaatgatggatgcaataaaaggaacaatgacagaaatatacaacgatctttctaaaaacactactggaagcacaatagctgagattcgcaggctgaggatcgagatagagaagctccagtggctgcaccagcaagagctctccgaaatgaaacacaacttagagctgaccatggcggagatgcggcagagcctggagcaggagcgggaccggctcatcgccgaggtgaagaagcagctggagttggagaagcagcaggcggtggatgagaccaagaagaagcagtggtgcgccaactgcaagaaggaggccatcttttactgctgttggaacactagctactgtgactacccctgccagcaagcccactggcctgagcacatgaagtcctgcacccagtcagctactgctcctcagcaggaagcggatgctgaggtgaacacagaaacactaaataagtcctcccaggggagctcctcgagcacacaatcagcaccttcagaaacggccagcgcctccaaagagaaggagacgtcagctgagaaaagcaaggagagtggctcgacccttgacctttctggctccagagagacgccctcctccattctcttaggctccaaccaaggctctgaccattcccggagtaataaatccagttggagcagcagtgatgagaagaggggatcgacacgttccgatcacaacaccagtaccagcacgaagagcctcctcccgaaagagtctcggctggacaccttctgggactagcagtgaatcgggacacaaaccacccaccccattgggagaaaaacccagacgccaggaaaagaagaaacaacaaaggcaggagaacagccactttcagacttgaaaatgacaaaaccctcagttgagcctgagcccccggcgcgggggctgctacactacaggacacccagcatcggctttgactgcagactgttcacccacacgagccctgtgcttttggtgtaaataatgtacaatttgtggatgtcattgaatctagaggactttcccctttttatatttgtattaactttaacttattaaaaaaaaaaaaaaaaaaa

SEQ ID NO: 16 - DNA, 2830 bp, Homo sapiens
aattcggcacgaggagagctggttgcgtgagtctcctcagctctgcttaccggtgcgactagcggcagcgacgcggctaaaagcgaaggggcgagtgcgagtcccctgagctgtacgaacgcggtcgccatggaccgcccagatgaggggcctccggccaagacccgccgcctgagcagctccgagtctccacagcgcgacccgcccccgccgccgccgccgccgccgctcctccgactgccgctgcctccaccccagcagcgcccgaggctccaggaggaaacggaggcggcacaggtgctggccgatatgaggggggtgggactgggccccgcgctgcccccgccgcctccctatgtcattctcgaggagggggggatccgcgcatacttcacgctcggtgctgagtgtcccggctgggattctaccatcgagtcggggtatggggaggcgcccccgcccacggagagcctggaagcactccccactcctgaggcctcgggggggagcctggaaatcgattttcaggttgtacagtcgagcagttttggtggagagggggccctagaaacctgtagcgcagtggggtgggcgccccagaggttagttgacccgaagagcaaggaagaggcgatcatcatagtggaggatgaggatgaggatgagcgggagagtatgaggagcagcaggaggcggcggcggcggcggaggaggaagcagaggaaggtgaagagggaaagcagagagagaaatgccgagaggatggagagcatcctgcaggcactggaggatattcagctggatctggaggcagtgaacatcaaggcaggcaaagccttcctgcgtctcaagcgcaagttcatccagatgcgaagacccttcctggagcgcagagacctcatcatccagcatatcccaggcttctgggtcaaagcattcctcaaccaccccagaatttcaattttgatcaaccgacgtgatgaagacattttccgctacttgaccaatctgcaggtacaggatctcagacatatctccatgggctacaaaatgaagctgtacttccagactaacccctacttcacaaacatggtgattgtcaaggagttccagcgcaaccgctcaggccggctggtgtctcactcaaccccaatccgctggcaccggggccaggaaccccaggcccgtcgtcacgggaaccaggatgcgagccacagctttttcagctggttctcaaaccatagcctcccagaggctgacaggattgctgagattatcaagaatgatctgtgggttaaccctctacgctactacctgagagaaaggggctccaggataaagagaaagaagcaagaaatgaagaaacgtaaaaccaggggcagatgtgaggtggtgatcatggaagacgcccctgactattatgcagtggaagacattttcagcgagatctcagacattgatgagacaattcatgacatcaagatctctgacttcatggagaccaccgactacttcgagaccactgacaatgagataactgacatcaatgagaacatctgcgacagcgagaatcctgaccacaatgaggtccccaacaacgagaccactgataacaacgagagtgctgatgaccacgaaaccactgacaacaatgagagtgcagatgacaacaacgagaatcctgaagacaataacaagaacactgatgacaacgaagagaaccctaacaacaacgagaacacttacggcaacaacttcttcaaaggtggcttctggggcagccatggcaacaaccaggacagcagcgacagtgacaatgaagcagatgaggccagtgatgatgaagataatgatggcaacgaaggtgacaatgagggcagtgatgatgatggcaatgaaggtgacaatgaaggcagcgatgatgacgacagagacattgagtactatgagaaagttattgaagactttgacaaggatcaggctgactacgaggacgtgatagagatcatctcagacgaatcagtggaagaagagggcattgaggaaggcatccagcaagatgaggacatctatgaggaaggaaactatgaggaggaaggaagtgaagatgtctgggaagaaggggaagattcggacgactctgacctagaggatgtgcttcaggtcccaaacggttgggccaatccggggaagagggggaaaaccggataagggttttccccttttggggatcacctctctgtatcccccacccactatcccatttgccctcctcctcagctagggccacgcggccccacattgcacttctggggggtgaccgacttcgtacacgggtttaaagtttatttttatggtttagtcattgcagagttcttattttggggggagggaaagggggctagtccccttcttttggccctccgcccccgcaggcttctgtgtgctgctaactgtatttattgtgatgccttggtcagggcccctctacccacttctcccagtcagttgtggccccagcccctctccctgtgctgtgtggagtggacaccctgacccccgaagcggggagggccgctgtggccttcgtcacagccgcgcagtgcccatggaggcgctgctgccaccttcctctcccaagttctttctccatccctctcctcttcccgccgcgccgctagcccgcctcggtgtctatgcaaggccgcttcgccattgcggtattctttgcggtattcttgtccccgtcccccagaaggctcgcctctccccgtggaccctgttaatcccaataaaattctgagcaagttcaaaaaaaaaaaaaaaaaaa

SEQ ID NO: 17 - DNA, 2100 bp, Homo sapiens
agagaatccagaaagtgatggagagccagtagtggaagatggaacttctgtaaaaacactggaaacactccagcaaagagtgaagcgtcaagagaacctacttaagcgttgtaaggaaacaattcagtcacataaggaacaatgtacactattaactagtgaaaaagaagctctgcaagaacaactggatgaaagacttcaagaactagaaaagataaaggaccttcatatggccgagaagactaaacttatcactcagttgcgtgatgcaaagaacttaattgaacagcttgaacaagataagggaatggtaatcgcagagacaaaacgtcagatgcatgaaaccctggaaatgaaagaagaagaaattgctcaactccgtagtcgcatcaaacagatgactacccagggagaggaattacgggaacagaaagaaaagtccgaaagagctgcttttgaggaacttgaaaaagctttgagtacagcccaaaaaacagaggaagcacggagaaaactgaaggcagaaatggatgaacaaataaaaactatcgaaaaaacaagtgaggaggaacgcatcagtcttcaacaggaattaagtcgggtgaaacaggaggttgttgatgtaatgaaaaaatcctcagaagaacaaattgctaagctacagaagcttcatgaaaaggagctggccagaaaagagcaggaactgaccaagaagcttcagacccgagaaagggaatttcaggaacaaatgaaagtagctcttgaaaagagtcaatcagaatatttgaagatcagccaagaaaaagaacagcaagaatctttggccctagaagagttagagttgcagaaaaaagcaatcctcacagaaagtgaaaataaacttcgggaccttcagcaagaagcagagacttacagaactagaattcttgaattggaaagttctttggaaaaaagcttacaagaaaacaaaaatcagtcaaaagatttggctgttcatctggaagctgaaaaaaataagcacaatatggagattacagtcatggttgaaaaacacaagacagaattggaaagccttaagcatcagcaggatgccctttggactgaaaaactccaagtcttaaagcaacaatatcagactgaaatggaaaaacttagggaaaagtgtgaacaagaaaaagaaacattgttgaaagacaaagagattatcttccaggcccacatagaagaaatgaatgaaaagactttagaaaagcttgatgtgaagcaaacagaactagaatcattatcttctgaactgtcagaagtattaaaagcccgtcacaaactagaagaggaactttctgttctgaaagatcaaacagataaaatgaagcaggaattagaggccaagatggatgaacagaaaaatcatcaccagcagcaagttgacagtatcattaaagaacacgaggtatctatccagaggactgagaaggcattaaaagatcaaattaatcaacttgagcttctcttgaaggaaagggacaagcatttgaaagagcatcaggctcatgtagaaaatttagaggcagatattaaaaggtctgaaggggaactccagcaggcatctgctaagctggacgtttttcagtcttaccagagtgccacacatgagcagacaaaagcatatgaggaacagttggcccaattgcagcagaagttgttggatttggaaacagaaagaattcttcttaccaaacaggttgctgaagttgaagcacaaaagaaagatgtttgtactgagttagatgctcacaaaatccaggtgcaggacttaatgcagcaacttgaaaaacaaaatagtgaaatggagcaaaaagtaaaatctttaacccaagtctatgagtccaaacttgaagatggtaacaaagaacaggaacagacaaagcaaatcttggtggaaaaggaaaatatgattttacaaatgagagaaggacagaagaaagaaattgagatactcacacagaaattgtcagccaaggaggacagtattcatattttgaatgaggaatatgaaaccaaatttaaaaaaaaaaaaaaaaaaaaaaaaaaaaaaaaaaa

SEQ ID NO: 18 - DNA, 5629 bp, Homo sapiens
cttaagtgcaaggaactctgtgttgggaggaaaaatgtccttcttcaatttccgtaagatcttcaagttggggagcgagaagaagaagaagcagtacgaacacgtgaagagggacctgaaccccgaagacttttgggagattataggagaactgggcgacggagcctttgggaaagtgtacaaggcccagaataaagagaccagtgttttagctgctgcaaaagtgattgacactaaatctgaagaagaacttgaagattacatggtagagattgacatattagcatcttgtgatcacccaaatatagtcaagcttctagatgccttctattatgagaacaatctttggatcctcattgaattttgtgcaggtggagcagtagatgctgtgatgcttgaacttgagagaccattaactgagtcccaaatacaagtagtttgcaagcagactttagatgcattgaactacttacatgataataagatcatccacagagatctgaaggctggcaacattctctttaccttagatggagatatcaaattggcggattttggagtatcagctaaaaacacgaggacaattcaaagaagagattcctttattggtacaccatattggatggctcctgaagtagtcatgtgtgaaacatctaaggacagaccctatgactacaaagctgatgtttggtccctgggtatcactttaatagaaatggctgagatagaaccacctcatcatgaattaaatccaatgcgagtgctgctaaaaatagcaaaatctgagccacctacattagcacagccatccagatggtcttcaaattttaaggactttctaaagaaatgcttagaaaagaatgtggatgccaggtggactacatctcagctgctgcagcatccctttgttactgttgattccaacaaacccatccgagaattgattgcagaggcgaaggctgaagtaacagaagaagttgaagatggcaaagaggaagatgaagaggaggaaacagaaaattctctgccaatacctgcaagtaagcgtgcatcttctgaccttagtatcgccagctctgaagaagataaactttcacaaaatgcttgtattttggagtctgtctcagaaaaaacagaacgtagtaactctgaagataaactcaacagcaaaattcttaatgaaaaacccaccactgatgaacctgaaaaggctgtggaggatattaatgaacatattaccgatgctcagttagaagcaatgactgaactccatgacagaacagcagtaatcaaggagaatgaaagagagaagaggcccaagcttgaaaatctgcctgacacagaagaccaagaaactgtggacattaattcagtcagtgaaggaaaagagaataatataatgataaccttagaaacaaatattgaacataatctaaaatctgaggaagaaaaggatcaggaaaagcaacagatgtttgaaaataagcttataaaatctgaagaaattaaagatactattttgcaaacagtagatttagtttctcaagagactggagaaaaagaggcaaatattcaggcagttgatagtgaagttgggcttacaaaggaagacacccaagagaaattgggggaagacgacaaaactcaaaaagatgtgatcagcaatacaagtgatgtgataggaacatgtgaggcagcagatgtggctcagaaagtggatgaagacagtgctgaggatacgcagagtaatgatgggaaagaagtggtcgaagtaggccagaaattaattaataagcccatggtgggtcctgaggctggtggtactaaggaagttcctattaaagaaatagttgaaatgaatgaaatagaagaaggtaaaaataaggaacaagcaataaacagttcagagaacataatggacatcaatgaggaaccaggaacaactgaaggtgaagaaatcactgagtcaagtagcactgaagaaatggaggtcagaagtgtggtggctgatactgaccaaaaggctttaggaagtgaagttcaggatgcttctaaagtcactactcagatagataaagagaaaaaagaaattccagtgtcaattaaaaaagagcctgaagttactgtagtttcacagcccactgaacctcagcctgttctaatacccagtattaatatcaactctgacagtggagaaaataaagaagaaataggttctttatcaaaaactgaaactattctgccaccagaatctgagaatccaaaggaaaatgataatgattcaggcactggttccactgctgatactagcagtattgacttgaatttatccatctctagctttctaagtaaaactaaagacagtggatcgatatctttacaagaaacaagaagacaaaagaaaacattgaagaaaacacgcaaatttattgttgatggtgtagaagtgagtgtaacaacatcaaagatagttacagatagtgattccaaaactgaagaattgcggtttcttagacgtcaggaacttcgggaattaagatttcttcagaaagaagagcaaagagcccaacaacagctcaatagcaaactacagcaacaacgagaacaaattttccggcgctttgagcaggaaatgatgagtaaaaagcgacaatatgaccaggaaattgagaatctagaaaaacagcagaaacagactatcgaacgcctggaacaagagcacacaaatcgcttgcgagatgaagccaaacgcatcaaaggagaacaagagaaagagttgtccaaatttcagaatatgctgaagaaccgaaagaaggaggttataaatgaagtggagaaagcacccaaagagctgagaaaagagctcatgaaacgcaggaaagaggagcttgcacaaagccagcatgctcaggaacaagagtttgttcagaaacaacagcaagaattagatggctctctgaaaaagatcatccagcagcagaaggcagagttagctaatattgagagagagtgcctgaataacaagcaacagctcatgagagctcgagaagctgcaatttgggagctcgaagaacgacacttacaagaaaaacaccagctgctcaaacagcagcttaaagatcagtatttcatgcaaagacatcagctacttaagcgccacgagaaggaaacagagcaaatgcagcgttacaatcaaagacttattgaggaattgaaaaacagacagactcaagaaagagcaagactgcccaagattcagcgcagtgaagccaagactcgaatggccatgtttaagaagagtttgagaattaactcaacagccacaccagatcaggaccgtgataaaattaaacagtttgctgcacaagaagaaaagaggcagaaaaatgagagaatggctcagcatcagaaacatgagaatcaaatgcgagatcttcagttgcagtgtgaagccaatgtccgcgaactgcatcagctgcagaatgaaaaatgccacttgttggttgagcatgagactcagaaactgaaggagttagatgaggaacatagccaagaattaaaggagtggagagagaaattgagacctaggaaaaagacactggaagaagagtttgccaggaaactacaggaacaggaagtattctttaaaatgactggggagtctgaatgccttaacccatcaacacagagccggatttccaaattttatcctattcccagcttgcattccaccggatcataacaaagggaagcattctgtgcgtgggtttggctctttcagtatgtcattctgttctcatcttctgccacagtctctcagatagctcatgaagacaatcacctgcctcaccttctaggtgttttccttttttgttttttttgttttgttttgtttttaagcaaagatgaagggaaaacgaactaagacagacgctaggccatgttggcaaagtagcatcttggtgactaaggtgactttgtatattcatcttaaaaattatgttctttagacactgctacctgaaaactgttggagaaataatgtttaaagttatttaagaaaaactgttacatcactaagtattaataaattcttcttacctgacgtaacttctcaatgcctaaattctgtagttgaagctctgctgcagagagttgggataattttcttttggtggatcagctctcataaaaaagctatgatttgctcaaatatgctgttgactcagtaaatgaatatatttttttctttaaataggaacaacctcttttaaaagagaaaaattatttcagtgatttgtcaaaacgaattacctcttttggcatgagctaataattgagggtgctaattttcttaagatagtgcctaaaacactaaatttcagtcaagtcgtaagtaggattttctttttgatcaacagggacaaaaacatctttagaattaaaaacatggttgttttggaatttttgcttctcttaccgtttgatagaaattttcatcctaaaatacatgtacaaagtttggaaagatgaaaaaaagaggtagcttttagattgcaaattggaaatgtaaaactcatgaaatttaagcaatataggtttagctatctgtgtttattttctaaaataatacctgagctggttaaatgatttctctccatcttagctaattctgtttaaaactctgtcagaggcctgcaggctgtgagttatatttataaatatatcttcagaaattaatcttaaaagaggcattagttcagaatacttttttaaaagtttaaattaaatatttaggcacgtcagaaattacttttccttattttgaaatgaggctacttatgtcttggttttattttgttccatgtttaaatcattcactttgatttgagtgggaaaagcctgaagcctttatcatgtggttgctggtgtgtgtaattattaatgaaatgttcactcctagtcccttatgaggcttagaatttcaaccacgtgtcaggtcagacagtattataaactgtactttgctgtctgagacagcacatttgtgaatgatgcttgctgcctgccattttcaacctattctctcttaagagtgctaggtaccaaattgtgaaagtttgttttcagttatattacttttgaggctggtgaaaaatttaaatgtaactttgtgggaacactgattcatatttagaaaatgtaaatgtctgtagcactttcttgcagttaatttgaaaactttggatgctgaaccttgtttgtcagtgatttagatgatttaaaaatgcatgtgtgatttgaattttataattgttttgacaagcataatttacttggacaacttcgtaggtagccttaacttctggccaagtttgttttttatataaatatatatacatatatacatattatgtatggttgtaaattcatacacttatcacatgaatgtgttactgtatacaaaactcttaatgctttattctcaaatgctgggttgaaaaatgttttgaaagccttttaaaatatatatctttataaagtaatattcaggatgatgataaaaattgtttatattgttatgataaaaatgacagtataatgttaaaaaaaaaaaaaaaaaaaaaaa

SEQ ID NO: 19 - DNA, 1991 bp, Homo sapiens
aaaacgctttttgcatacaagcaggaaaatgagatgttatccagtagtagagatcagagagttgtgacatctgaggaccaagttcaagaagggactaaagtgctgaaacttaaaacaaaaatggctgataaagaaaacatgaagagacctgcagagagcaaaaataatacagtggtggggaaacattgtattcctttaaaaccttcaaatgaactaaccaattcaactgtagtaattgacacacataaacctaaggatagtaatcaaactccgcatttgttactaactgaagatgatccccaaagtcaacatatgacattaagccaggcatttcaccttaaaaacaatagtaaaaagaaacaaatgactacagaaaaacaaaagcaagatgctaacatgcccaagaaacctgtgcttggatcttatcgtggccagattgttcagtctaagattaattcatttagaaaacctctacaagtcaaagatgagagttctgcagcaacaaagaaactttcagccactatacctaaagccacaaaacctcagcctgtaaacaccagcagtgtaacagtgaaaagtaatagatcctccaataagactgccactactaaatttgtgagcactacatctcagaacacacaacttgtgcgacctcctattagaagtcatcacagtaatacccgggacactgtgaaacaaggcatcagtagaacctctgccaatgttacaatccggaaagggcctcatgaaaaagaactattacaatcaaaaacagctttatctagtgtcaaaaccagttcttctcaaggtataataagaaataagactctatcaagatccatagcatctgaagttgtagccaggcctgcttcattgtctaatgataaactgatggaaaagtcagagcccgttgaccagcgaagacatactgcaggaaaagcaattgttgatagtagatcagctcagcccaaagaaacctcggaagagagaaaagctcgtctgagtgagtggaaagctggcaaaggaagagtgctaaaaaggccccctaattcagtagttactcagcatgagcctgcaggacaaaatgaaaaaccagttgggtctttttggactaccatggcagaagaagatgaacaaagattatttactgaaaaagtaaacaacacattttctgaatgcctgaacttgattaatgagggatgtccaaaagaagatatactggtcacactgaatgacctgattaaaaatattccagatgccaaaaagcttgttaagtattggatatgtcttgcacttattgaaccaatcacaagtcctattgaaaatattattgcaatctatgagaaagccattctggcaggggctcagcctattgaagagatgcgacacacgattgtagatattctaacaatgaagagtcaagaaaaagctaatttaggagaaaatatggagaagtcttgtgcaagcaaggaagaagtcaaagaagtcagtattgaagatacaggtgttgatgtagatccagaaaaactggaaatggagagtaaacttcatagaaatttgctatttcaagattgtgaaaaagagcaagacaacaaaacaaaagatccaacccatgatgttaaaacccccaatacagaaacgaggacaagttgcttaattaaatataatgtgtctactacgccatacttgcaaagtgtgaaaaaaaaaggtgcagtttgatggaacaaattccgcatttaaagagctgaagtttttaacaccagtgagacgttctcgacgtcttcaagagaaaacttctaaattgccagatatgttaaaagatcattatccttgtgtgtcttcattggaacagctaacggagttgggaagagaaactgatgcttttgtatgccgccctaatgcagcactgtgccgggtgtactatgaggctgatacaacataagagaaataaagctctgttagggaaaaaaaaaaaaaaaaaaaaaaaaaaa

SEQ ID NO: 20 - DNA, 3462 bp, Homo sapiens
cagatgtattaaaaatagctcagtttttacaattttctttgattcagtgtcgaaaggaattcaaaaatataagcgccataaattttcattctgttgttgaaaagtatgtaagtgaattttttaagcgaggttttggttcaggtaaacgagagtttattatgtttccatatgattcacgattagatgataaaaaattcttatactcagctcccagaaataaatcccatattgatacttgtttgcatgcctatatttttcggcctgaagtgtatcagttacctatttgtaaattaaaagaactatttgaagaaaatagaaaacttcagcagtttagtccactttcagattatgaaggtcaagaagaagaaatgaatggtacaaaaatgaaatttggaaaacgaaataactcaagaggtgaagccattatatctggaaagcaaagatcatctcattctttggattatgataaggatagagtcaaagaattgattaatttaattcagtgtaggaaaaagagtgtgggtggggactcagacacagaagatatgagaagcaaaactgtcttgaagaggaagcttgaggatctacctgaaaatatgagaaagctcgccaaaaccagtaatttatctgaaaattgccatctgtatgaagagtctccacagcctattggctcacttggacatgatgctgacttgaggcggcagcagcaggatacctgtaactccggcattgctgacatccataggctgtttaattggttatcagaaacactagcaaatgcgcgccattctgatgcatctctgacagacacagtcaacaaagccttaggattgagcactgatgatgcctatgaagagctgaggcaaaaacatgagtatgagttgaactctaccccagataagaaagactatgagcagcctacttgtgcaaaagttgaaaatgcacagtttaagggtactcagagcttattactagaagttgatgcaacatctaagtattctgttgctatttctaccagcgaagtgggcactgaccataagctacatttgaaagaagatccaaatttaattagcgtgaataattttgaagattgcagtttgtgtcccagtgttcccattgaacatggatttcgtagacaacagtctaagtcaaataatgttgaagagactgaaatacattggaaactgattccaattacagacacactaaagggcaccactgaggatgacgtgttgacaggtcaggtggaggagcagtgtgtgccagcagcagaggcagagccgcctgcagtgagcgaaaccacagagaggacagtgttaggagagtacaatctcttttctaggaagatagaagagattttgaagcaaaagaatgtttcatatgtcagtagagtttccacacctatcttttcaacacaagagaagatgaaacggctttccgagttcatatattctaagacttccaaagctggtgtgcaggagtttgtagatggtttgcatgagaagctaaatactattattattaaagcatcagccaagggtgggaatttgccaccagtcagtcctaacgattctggtgctaagatagcatcgaatcctctggaaaggcatgtcataccagtttcctcaagtgacttcaacaataaacatctccttgagccactgtgtagtgatcctttgaaagataccaactctgatgagcagcattccacttcggctttaactgaagtagaaatgaaccagcctcaacatgccacagagttaatggtgacttctgatcatattgtacctggtgatatggcccgggaaccagtagaagaaacaacaaaatcccccagtgatgtaaacatttctgctcagccagctctttcaaattttataagccagttagaacctgaagtatttaatagtttggttaaaatcatgaaagacgtccagaaaaatactgtgaaattttatattcatgaagaagaagagagtgtgctctgtaaagaaataaaggaatatcttatcaaattaggcaatacagaatgtcatcctgaacagtttttggaaagaagatcaaaattagataaactattgattattattcaaaatgaagacattgcaggtttcattcacaagatacctggcttggtgactttaaagaagctcccctgtgttagttttgctggtgttgatagcctggatgatgttaaaaatcatacatacaatgaattatttgtatctggaggttttatcgtatctgatgaatcaattctaaacccagaggttgtcacagttgagaaccttaaaaattttttgacattccttgaggaacttagtactccagaaggaaaatggcaatggaaagtccactgtaaatttcagaagaaactaaaggaactaggcagattgaatgctaaagctctaagtctgttgacgcttctgaatgtctatcagaagaaacatctggttgaaattttgtcataccacaattgtgattcacaaactcgaaatgctccagaattggattgccttatcagacttcaggctcagaacatacagcaacgacacgtagtctttttaacagagaagaacatcaagatgctttccagttatacagataatggaatagtggttgcaactgctgaagacttcatgcaaaactttaaaaatcttgtgggctatcacaattcaatcacagaagaaaaccttccacagcttggtgctaatgagaatcttgagtcgcagtcagctcttttagaaaacgatgaaaaggatgaagaggatatgtctctggattcaggggatgaaatctcacatatagaagtatgcagcaattttcattcagaaatatgggagaaagagaccaaaggatcacgtggaacagatcaaaaaaagaatactcaaattgagttgcaatcgtctcctgatgtgcaaaacagtttattagaagataagacttaccttgattctgaagagagaacttctattgatatagtatgctctgaaggagagaacagcaattcaacagaacaagattcatatagtaactttcaggtttatcatagtcaattaaatatgtcccatcagtttagtcattttaatgttctcactcatcagacatttttggggacaccatatgccctttcatcaagtcagtctcaagaaaatgagaattacttcttatctgcttatactgaaagcttggatagagataaatctccacctcccttaagttgggggaaaagtgattcttccaggccatattcacaagagaaataactgtagtaactttttttttaagagattgttgtggactttgtttattaacaatttatatttcattctctaaacaaaaggttcttgttctttctcaaatgttttttcttttatttaaatcatgatggcctgtaacagttgaagcatctaaaaattgaaataaatatatatttttaacataaaaaaaaaaaaaaaaaaa

SEQ ID NO: 21 - DNA, 1368 bp, Homo sapiens
aattcggcacgaggggagcgcagcagccatggcaagccgtctcctgctcaacaacggcgccaagatgcccatcctggggttgggtacctggaagtcccctccagggcaggtgactgaggccgtgaaggtggccattgacgtcgggtaccgccacatcgactgtgcccatgtgtaccagaatgagaatgaggtgggggtggccattcaggagaagctcagggagcaggtggtgaagcgtgaggagctcttcatcgtcagcaagctgtggtgcacgtaccatgagaagggcctggtgaaaggagcctgccagaagacactcagcgacctgaagctggactacctggacctctaccttattcactggccgactggctttaagcctgggaaggaatttttcccattggatgagtcgggcaatgtggttcccagtgacaccaacattctggacacgtgggcggccatggaagagctggtggatgaagggctggtgaaagctattggcatctccaacttcaaccatctccaggtggagatgatcttaaacaaacctggcttgaagtataagcctgcagttaaccagattgagtgccacccatatctcactcaggagaagttaatccagtactgccagtccaaaggcatcgtggtgaccgcctacagccccctcggctctcctgacaggccctgggccaagcccgaggacccttctctcctggaggatcccaggatcaaggcgatcgcagccaagcacaataaaactacagcccaggtcctgatccggttccccatgcagaggaacttggtggtgatccccaagtctgtgacaccagaacgcattgctgagaactttaaggtctttgactttgaactgagcagccaggatatgaccaccttactcagctacaacaggaactggagggtctgtgccttgttgagctgtacctcccacaaggattaccccttccatgaagagttttgaagctgtggttgcctgctcgtccccaagtgacctatacctgtgtttcttgcctcatttttttccttgcaaatgtagtatggcctgtgtcactcagcagtgggacagcaacctgtagagtggccagcgagggcgtgtctagcttgatgttggatctcaagagccctgtcagtagagtagaagtctcttccagtttgctttgcccttctttctaccctgctggggaaagtacaacctgaatacccttttctgaccaaagagaagcaaaatctaccaggtcaaaatagtgccactaacggttgagttttgactgcttggaactggaatcctttcagcaagacttctctttgcctcaaataaaaagtgcttttgtgaaaaaaaaaaaaaaaaaa

SEQ ID NO: 22 - DNA, 398 bp, Homo sapiens
  modified_base (1): a, t, c, g, other or unknown
ngthctbrtynchckaattcggcacgaggctgcggacataaatcttaaagctagtaacatgttgttcttctaggaattccattcagctacagatttaaggtttatcagtagtatttccagaaagatggtccgacacagtggctcacgtttataatcccagcactttgggaggccgaggtgggtgaattgcttgagtccaggagttcaagaccagcctgggcaacatggcaaaaccctgtctttgcctgtagtacccccagctatttgagaggctgaggtggaagaatcacctgagcctggggaggtcagggctgcagtgtgctgaaattgcacaactgcactccagcctgggcaatcagagtgagaccctgtctttaagaaaaaaaaaaaaaaaaaaa

SEQ ID NO: 23 - protein, 685 aa, Homo sapiens [sequence omitted]

SEQ ID NO: 24 - protein, 1075 aa, Homo sapiens [sequence omitted]

SEQ ID NO: 25 - protein, 852 aa, Homo sapiens
MKRAAAKHLIERYYHQLTEGCGNEACTNEFCASCPTFLRMDNNAAAIKALELYKINAKLCDPHPSKKGASSAYLENSKGAPNNSCSEIKMNKKGARIDFKDVTYLTEEKVYEILELCREREDYSPLIRVIGRVFSSAEALVQSFRKVKQHTKEELKSLQAKDEDKDEDEKEKAACSAAAMEEDSEASSSRIGDSSQGDNNLQKLGPDDVSVDIDAIRRVYTRLLSNEKIETAFLNALVYLSPNVECDLTYHNVYSRDPNYLNLFIIVMENRNLHSPEYLEMALPLFCKAMSKLPLAAQGKLIRLWSKYNADQIRRMMETFQQLITYKVISNEFNSRNLVNDDDAIVAASKCLKMVYYANVVGGEVDTNHNEEDDEEPIPESSELTLQELLGEERRNKKGPRVDPLETELGVKTLDCRKPLIPFEEFINEPLNEVLEMDKDYTFFKVETENKFSFMTCPFILNAVTKNLGLYYDNRIRMYSERRITVLYSLVQGQQLNPYLRLKVRRDHIIDDALVRLEMIAMENPADLKKQLYVEFEGEQGVDEGGVSKEFFQLVVEEIFNPDIGMFTYDESTKLFWFNPSSFETEGQFTLIGIVLGLAIYNNCILDVHFPMVVYRKLMGKKGTFRDLGDSHPVLYQSLKDLLEYEGNVEDDMMITFQISQTDLFGNPMMYDLKENGDKIPITNENRKEFVNLYSDYILNKSVEKQFKAFRRGFHMVTNESPLKYLFRPEEIELLICGSRNLDFQALEETTEYDGGYTRDSVLIREFWEIVHSFTDEQKRLFLQFTTGTDRAPVGGLGKLKMIIAKNGPDTERLPTSHTCFNVLLLPEYSSKEKLKERLLKAITYAKGFGML

SEQ ID NO: 26 - protein, 335 aa, Homo sapiens
MTTVTVTTEIPPRDKMEDNSALYESTSAHIIEETEYVKKIRTTLQKIRTQMFKDEIRHDSTNHKLDAKHCGNLQQGSDSEMDPSCCSLDLLMKKIKGKDLQLLEMNKENEVLKIKLQASREAGAAALRNVAQRLFENYQTQSEEVRKKQEDSKQLLQVNKLEKEQKLKQHVENLNQVAEKLEEKHSQITELENLVQRMEKEKRTLLERKLSLENKLLQLKSSATYGKSCQDLQREISILQEQISHLQFVIHSQHQNLRSVIQEMEGLKNNLKEQDKRIENLREKVNILEAQNKELKTQVALSSETPRTKVSKAVSTSELKTEGVSPYLMLIRLRK

SEQ ID NO: 27 - protein, 254 aa, Homo sapiens
MLISKLEKNKTMKSEDKAEIMKTLEVLTKNITKLKDEVKAASPGRCLPKSIKTKTQMQKELLDTELDLYKKMQAGEEVTELRRKYTELQLEAAKRGILSSGRGRGIHSRGRGAVHGRGRGRGRGRGVPGHAVVDHRPRALEISAFTESDREDLLPHFAQYGEIEDCQIDDSSLHAVITFKTRAEAEAAAVHGARFKGQDLKLAWNKPVTNISAVETEEVEPDEEEFQEESLVDDSLLQDDDEEEEDNESRSWRR

SEQ ID NO: 28 - protein, 1035 aa, Homo sapiens [sequence omitted]

SEQ ID NO: 29 - protein, 613 aa, Homo sapiens
MNKGEHALVLFEKCVQDKYLQQEHIIKKLIKENKKHQELFVDICSEKDNLREELKKRTETEKQHMNTIKQLESRIEELNKEVKASRDKLIAQDVTAKNAVQQLHKEMAQRMEQANKKCEEARQEKEAMVMKYVRGEKESLDLRKEKETLEKKLRDANKELEKNTNKIKQLSQEKGRLHQLYETKEGETTRLIREIDKLKEDINSHVIKVKWAQNKLKAEMDSHKETKDKLKETTTKLTQAKEEADQIRKNCQDMIKTYQESEEIKSNELDAKLRVTKGELEKQMQEKSDQLEMHHAKIKELEDLKRTFKEGMDELRTLRTKVKCLEDERLRTEDELSKYKEIINRQKAEIQNLLDKVKTADQLQEQLQRGKQEIENLKEEVESLNSLINDLQKDIEGSRKRESELLLFTERLTSKNAQLQSESNSLQSQFDKVSCSESQLQSQCEQMKQTNINLESRLLKEEELRKEEVQTLQAELACRQTEVKALSTQVEELKDELVTQRRKHASSIKDLTKQLQQARRKLDQVESGSYDKEVSSMGSRSSSSGSLNARSSAEDRSPENTGSSVAVDNFPQVDKAMLIERIVRLQKAHARKNEKIEFMEDHIKQLVEKKKKK

SEQ ID NO: 30 - protein, 810 aa, Homo sapiens
MSKKGRNKGEKPEALIVALQAANEDLRTKLTDIQIELHQEKSKVSKLEREKTQEAKRIRELEQRKHTVLVTELKAKLHEEKMKELQAVRENLIKQHEQEMSRTVKVRDGEIQRLKSALCALRDGSSDKVRTALTIEAREEARKLFDTERLKLLQEIADLKTAKKQVDEALSNMIQADKIKAGDLRSEHQSHQEAISKIKWESERDIRRLMDEIKAKDRIIFSLEKELETQTGYVQKLQLQKEALDEQLFLVKEAECNMSSPKREIPGRAGDGSEHCSSPDLRRNQKRIAELNATIRKLEDRNTLLGDERNELLKRVRETEKQCKPLLERNKCLAKRNDELMVSLQRMEEKLKAVTKENSEMREKITSHPPLKKLKSLNDLDQANEEQETEFLKLQVIEQQNIIDELTRDREKLIRRRKHRRSSKPIKRPVLDPFIGYDEDSMDSETSSMASFRTDRTPATPDDDLDESLAAEESELRFRQLTKEYQALQRAYALLQEQTGGIIDAEREAKAQEQLQAEVLRYKAKIEDLEATLAQKGQDSHWVEDKQLFIKRNQELLEKIEKQEAENHRLQQELQDARDQNELLEFRNLELEERERRSPPFNLQIHPFSDGVSALQIYCMKEGVKDVNIPDLIKQLDILGDNGNLRNEEQVAIIQASTVLSLAEKWIQQIEGAEAALHQKMMELESDMEQFCKIKGYLEEELDYRKQALDQAYMRIQELEATLYNALQQETVIKFGELLSEKQQEELRTAVEKLRRQMLRKSREYDCQILQERMELLQQAHQRIRDLEDKTDIQKRQIKDLEEKSNRKHG

SEQ ID NO: 31 - protein, 387 aa, Homo sapiens [sequence omitted]

SEQ ID NO: 32 - protein, 300 aa, Homo sapiens
MKFLLDILLLLPLLIVCSLESFVKLFIPKRRKSVTGEIVLITGAGHGIGRLTAYEFAKLKSKLVLWDINKHGLEETAAKCKGLGAKVHTFVVDCSNREDIYSSAKKVKAEIGDVSILVNNAGVVYTSDLFATQDPQIEKTFEVNVLAHFWTTKAFLPAMTKNNHGHIVTVASAAGHVSVPFLLAYCSSKFAAVGFHKTLTDELAALQITGVKTTCLCPNFVNTGFIKNPSTSLGPTLEPEEVVNRLMHGILTEQKMIFIPSSIAFLTTLERILPERFLAVLKRKISVKFDAVIGYKMKAQ

SEQ ID NO: 33 - protein, 361 aa, Homo sapiens
MITGTSQADCAVLIVAAGVGEFEAGISKNGQTREHALLAYTLGVKQLIVGVNKMDSTEPPYSQKRYEEIVKEVSTYIKKIGYNPDTVAFVPISGWNGDNMLEPSANMPWFKGWKVTRKDGNASGTTLLEALDCILPPTRPTDKPLRLPLQDVYKIGGIGTVPVGRVETGVLKPGMVVTFAPVNVTTEVKSVEMHHEALSEALPGDNVGFNVKNVSVKDVRRGNVAGDSKNDPPMEAAGFTAQVIILNHPGQISAGYAPVLDCHTAHIACKFAELKEKIDRRSGKKLEDGPKFLKSGDAAIVDMVPGKPMCVESFSDYPPLGRFAVRDMRQTVAVGVIKAVDKKAAGAGKVTKSAQKAQKAK

SEQ ID NO: 34 - protein, 795 aa, Homo sapiens
MEKQKPFALFVPPRSSSSQVSAVKPQTLGGDSTFFKSFNKCTEDDFEFPFAKTNLSKNGENIDSDPALQKVNFLPVLEQVGNSDCHYQEGLKDSDLENSEGLSRVYSKLYKEAEKIKKWKVSTEAELRQKESKLQENRKIIEAQRKAIQELQFGNEKVSLKLEEGIQENKDLIKENNATRHLCNLLKETCARSAEKTKKYEYEREETRQVYMDLNNNIEKMITAFEELRVQAENSRLEMHFKLKEDYEKIQHLEQEYKKEINDKEKQVSLLLIQITEKENKMKDLTFLLEESRDKVNQLEEKTKLQSENLKQSIEKQHHLTKELEDIKVSLQRSVSTQKALEEDLQIATKTICQLTEEKETQMEESNKARAAHSFVVTEFETTVCSLEELLRTEQQRLEKNEDQLKILTMELQKKSSELEEMTKLTNNKEVELEELKKVLGEKETLLYENKQFEKIAEELKGTEQELIGLLQAREKEVHDLEIQLTAITTSEQYYSKEVKDLKTELENEKLKNTELTSHCNKLSLENKELTQETSDMTLELKNQQEDINNNKKQEERMLKQIENLQETETQLRNELEYVREELKQKRDEVKCKLDKSEENCNNLRKQVENKNKYIEELQQENKALKKKGTAESKQLNVYEIKVNKLELELESAKQKFGEITDTYQKEIEDKKISEENLLEEVEKAKVIADEAVKLQKEIDKRCQHKIAEMVALMEKHKHQYDKIIEERDSELGLYKSKEQEQSSLRASLEIELSNLKAELLSVKKQLEIEREEKEKLKREAKENTATLKEKKKKK

SEQ ID NO: 35 - protein, 761 aa, Homo sapiens
MEQTVAEQDDSLSSLLVKLKKVSQDLERQREITELKVKEFENIKLQLQENHEDEVKKVKAEVEDLKYLLDQSQKESQCLKSELQAQKEANSRAPTTTMRNLVERLKSQLALKEKQQKALSRALLELRAEMTAAAEERIISATSQKEAHLNVQQIVDRHTRELKTQVEDLNENLLKLKEALKTSKNRENSLTDNLNDLNNELQKKQKAYNKILREKEEIDQENDELKRQIKRLTSGLQGKPLTDNKQSLIEELQRKVKKLENQLEGKVEEVDLKPMKEKNAKEELIRWEEGKKWQAKIEGIRNKLKEKEGEVFTLTKQLNTLKDLFAKADKEKLTLQRKLKTTGMTVDQVLGIRALESEKELEELKKRNLDLENDILYMRAHQALPRDSVVEDLHLQNRYLQEKLHALEKQFSKDTYSKPSISGIESDDHCQREQELQKENLKLSSENIELKFQLEQANKDLPRLKNQVRDLKEMCEFLKKEKAEVQRKLGHVRGSGRSGKTIPELEKTIGLMKKVVEKVQRENEQLKKASGILTSEKMANIEQENEKLKAELEKLKAHLGHQLSMHYESKTKGTEKIIAENERLRKELKKETDAAEKLRIAKNNLEILNEKMTVQLEETGKRLQFAESRGPQLEGADSKSWKSIVVTRMYETKLKELETDIAKKNQSITDLKQLVKEATEREQKVNKYNEDLEQQIKILKHVPEGAETEQGLKRELQVLRLANHQLDKEKAELIHQIEANKDQSGAESTIPDADQLKKKKK

SEQ ID NO: 36 - protein, 637 aa, Homo sapiens
MFRQAMRTPIIWFHVVPAANKEQYEQLSQSEKNNYYSSRFSPDSQYIDNRSVNSAGLHTVQRAPRLNHPPEQIDSHSRLPHSAHPSGKPPSAPASAPQNVFSTTVSSGYNTKKIGKRLNIQLKKGTEGLEFSITSRDVTIGGSAPIYVKNILPRGAAIQDGRLKAGDRLIEVNGVDLVGKSQEEVVSLLRSTKMEGTVSLLVFRQEDAFHPRELNAEPSQMQIPKETKAEDEDIVLTPDGTREFLTFEVPLNDSGSAGLGVSVKGNRSKENHADLGIFVKSIINGGAASKDGRLRVNDQLIAVNGESLLGKTNQDAMETLRRSMSTEGNKRGMIQLIVARRISKCNELKSPGSPPGPELPIETALDDRERRISHSLYSGIEGLDESPSRNAALSRIMGESGKYQLSPTVNMPQDDTVIIEDDRLPVLPPHLSDQSSSSSHDDVGFVTADAGTWAKAAISDSADCSLSPDVDPVLAFQREGFGRQIADETKLNTVDDQKAGSPSRDVGPSLGLKKSSSLESLQTAVAEVTLNGDIPFHRPRPRIIRGRGCNESFRAAIDKSYDKPAVDDDDEGMETLEEDTEESSRSGRESVSTASDQPSHSLERQMNGNQEKGDKTDRKKKKKKKKKKKKKKKKKKK

SEQ ID NO: 37 - protein, 764 aa, Homo sapiens [sequence omitted]

SEQ ID NO: 38 - protein, 623 aa, Homo sapiens
MAEKTKLITQLRDAKNLIEQLEQDKGMVIAETKRQMHETLEMKEEEIAQLRSRIKQMTTQGEELREQKEKSERAAFEELEKALSTAQKTEEARRKLKAEMDEQIKTIEKTSEEERISLQQELSRVKQEVVDVMKKSSEEQIAKLQKLHEKELARKEQELTKKLQTREREFQEQMKVALEKSQSEYLKISQEKEQQESLALEELELQKKAILTESENKLRDLQQEAETYRTRILELESSLEKSLQENKNQSKDLAVHLEAEKNKHNMEITVMVEKHKTELESLKHQQDALWTEKLQVLKQQYQTEMEKLREKCEQEKETLLKDKEIIFQAHIEEMNEKTLEKLDVKQTELESLSSELSEVLKARHKLEEELSVLKDQTDKMKQELEAKMDEQKNHHQQQVDSIIKEHEVSIQRTEKALKDQINQLELLLKERDKHLKEHQAHVENLEADIKRSEGELQQASAKLDVFQSYQSATHEQTKAYEEQLAQLQQKLLDLETERILLTKQVAEVEAQKKDVCTELDAHKIQVQDLMQQLEKQNSEMEQKVKSLTQVYESKLEDGNKEQEQTKQILVEKENMILQMREGQKKEIEILTQKLSAKEDSIHILNEEYETKFKKKKKKKKKKK

SEQ ID NO: 39 - protein, 1235 aa, Homo sapiens
MSFFNFRKIFKLGSEKKKKQYEHVKRDLNPEDFWEIIGELGDGAFGKVYKAQNKETSVLAAAKVIDTKSEEELEDYMVEIDILASCDHPNIVKLLDAFYYENNLWILIEFCAGGAVDAVMLELERPLTESQIQVVCKQTLDALNYLHDNKIIHRDLKAGNILFTLDGDIKLADFGVSAKNTRTIQRRDSFIGTPYWMAPEVVMCETSKDRPYDYKADVWSLGITLIEMAEIEPPHHELNPMRVLLKIAKSEPPTLAQPSRWSSNFKDFLKKCLEKNVDARWTTSQLLQHPFVTVDSNKPIRELIAEAKAEVTEEVEDGKEEDEEEETENSLPIPASKRASSDLSIASSEEDKLSQNACILESVSEKTERSNSEDKLNSKILNEKPTTDEPEKAVEDINEHITDAQLEAMTELHDRTAVIKENEREKRPKLENLPDTEDQETVDINSVSEGKENNIMITLETNIEHNLKSEEEKDQEKQQMFENKLIKSEEIKDTILQTVDLVSQETGEKEANIQAVDSEVGLTKEDTQEKLGEDDKTQKDVISNTSDVIGTCEAADVAQKVDEDSAEDTQSNDGKEVVEVGQKLINKPMVGPEAGGTKEVPIKEIVEMNEIEEGKNKEQAINSSENIMDINEEPGTTEGEEITESSSTEEMEVRSVVADTDQKALGSEVQDASKVTTQIDKEKKEIPVSIKKEPEVTVVSQPTEPQPVLIPSININSDSGENKEEIGSLSKTETILPPESENPKENDNDSGTGSTADTSSIDLNLSISSFLSKTKDSGSISLQETRRQKKTLKKTRKFIVDGVEVSVTTSKIVTDSDSKTEELRFLRRQELRELRFLQKEEQRAQQQLNSKLQQQREQIFRRFEQEMMSKKRQYDQEIENLEKQQKQTIERLEQEHTNRLRDEAKRIKGEQEKELSKFQNMLKNRKKEVINEVEKAPKELRKELMKRRKEELAQSQHAQEQEFVQKQQQELDGSLKKIIQQQKAELANIERECLNNKQQLMRAREAAIWELEERHLQEKHQLLKQQLKDQYFMQRHQLLKRHEKETEQMQRYNQRLIEELKNRQTQERARLPKIQRSEAKTRMAMFKKSLRINSTATPDQDRDKIKQFAAQEEKRQKNERMAQHQKHENQMRDLQLQCEANVRELHQLQNEKCHLLVEHETQKLKELDEEHSQELKEWREKLRPRKKTLEEEFARKLQEQEVFFKMTGESECLNPSTQSRISKFYPIPSLHSTGS

SEQ ID NO: 40 - protein, 561 aa, Homo sapiens
MLSSSRDQRVVTSEDQVQEGTKVLKLKTKMADKENMKRPAESKNNTVVGKHCIPLKPSNELTNSTVVIDTHKPKDSNQTPHLLLTEDDPQSQHMTLSQAFHLKNNSKKKQMTTEKQKQDANMPKKPVLGSYRGQIVQSKINSFRKPLQVKDESSAATKKLSATIPKATKPQPVNTSSVTVKSNRSSNKTATTKFVSTTSQNTQLVRPPIRSHHSNTRDTVKQGISRTSANVTIRKGPHEKELLQSKTALSSVKTSSSQGIIRNKTLSRSIASEVVARPASLSNDKLMEKSEPVDQRRHTAGKAIVDSRSAQPKETSEERKARLSEWKAGKGRVLKRPPNSVVTQHEPAGQNEKPVGSFWTTMAEEDEQRLFTEKVNNTFSECLNLINEGCPKEDILVTLNDLIKNIPDAKKLVKYWICLALIEPITSPIENIIAIYEKAILAGAQPIEEMRHTIVDILTMKSQEKANLGENMEKSCASKEEVKEVSIEDTGVDVDPEKLEMESKLHRNLLFQDCEKEQDNKTKDPTHDVKTPNTETRTSCLIKYNVSTTPYLQSVKKKGAV

SEQ ID NO: 41 - protein, 214 aa, Homo sapiens
MTKTLPGGIMVNGSRLKNLVLTYVNAISSGDLPCIENAVLALAQRENSAAVQKAIAHYDQQMGQKVQLPMETLQELLDLHRTSEREAIEVFMKNSFKDVDQSFQKELETLLDAKQNDICKRNLEASSDYCSALLKDIFGPLEEAVKQGIYSKPGGHNLFIQKTEELKAKYYREPRKGIQAEEVLQKYLKSKESVSHAILQTDQALTETEKKKKK

SEQ ID NO: 42 - protein, 693 aa, Homo sapiens
MDRPDEGPPAKTRRLSSSESPQRDPPPPPPPPPLLRLPLPPPQQRPRLQEETEAAQVLADMRGVGLGPALPPPPPYVILEEGGIRAYFTLGAECPGWDSTIESGYGEAPPPTESLEALPTPEASGGSLEIDFQVVQSSSFGGEGALETCSAVGWAPQRLVDPKSKEEAIIIVEDEDEDERESMRSSRRRRRRRRRKQRKVKRESRERNAERMESILQALEDIQLDLEAVNIKAGKAFLRLKRKFIQMRRPFLERRDLIIQHIPGFWVKAFLNHPRISILINRRDEDIFRYLTNLQVQDLRHISMGYKMKLYFQTNPYFTNMVIVKEFQRNRSGRLVSHSTPIRWHRGQEPQARRHGNQDASHSFFSWFSNHSLPEADRIAEIIKNDLWVNPLRYYLRERGSRIKRKKQEMKKRKTRGRCEVVIMEDAPDYYAVEDIFSEISDIDETIHDIKISDFMETTDYFETTDNEITDINENICDSENPDHNEVPNNETTDNNESADDHETTDNNESADDNNENPEDNNKNTDDNEENPNNNENTYGNNFFKGGFWGSHGNNQDSSDSDNEADEASDDEDNDGNEGDNEGSDDDGNEGDNEGSDDDDRDIEYYEKVIEDFDKDQADYEDVIEIISDESVEEEGIEEGIQQDEDIYEEGNYEEEGSEDVWEEGEDSDDSDLEDVLQVPNGWANPGKRGKTG

SEQ ID NO: 43 - protein, 316 aa, Homo sapiens
MASRLLLNNGAKMPILGLGTWKSPPGQVTEAVKVAIDVGYRHIDCAHVYQNENEVGVAIQEKLREQVVKREELFIVSKLWCTYHEKGLVKGACQKTLSDLKLDYLDLYLIHWPTGFKPGKEFFPLDESGNVVPSDTNILDTWAAMEELVDEGLVKAIGISNFNHLQVEMILNKPGLKYKPAVNQIECHPYLTQEKLIQYCQSKGIVVTAYSPLGSPDRPWAKPEDPSLLEDPRIKAIAAKHNKTTAQVLIRFPMQRNLVVIPKSVTPERIAENFKVFDFELSSQDMTTLLSYNRNWRVCALLSCTSHKDYPFHEEF

SEQ ID NO: 44 - DNA, 22 bp, Artificial Sequence
  Description of Artificial Sequence Primer
gcaaaagcaattagacgctacc

SEQ ID NO: 45 - DNA, 22 bp, Artificial Sequence
  Description of Artificial Sequence Primer
cacagccctgttcttctttagc

SEQ ID NO: 46 - DNA, 27 bp, Artificial Sequence
  Description of Artificial Sequence Primer
gtacagcagaaagcaagcaactgaatg

SEQ ID NO: 47 - DNA, 28 bp, Artificial Sequence
  Description of Artificial Sequence Primer
ggaaattggattctaaagcagttccttc

SEQ ID NO: 48 - DNA, 22 bp, Artificial Sequence
  Description of Artificial Sequence Primer
ctatgaatccaagaccaaaggc

SEQ ID NO: 49 - DNA, 22 bp, Artificial Sequence
  Description of Artificial Sequence Primer
ctccactttggtccttgttagc

SEQ ID NO: 50 - DNA, 20 bp, Artificial Sequence
  Description of Artificial Sequence Primer
acccacgcagatttggaatc

SEQ ID NO: 51 - DNA, 19 bp, Artificial Sequence
  Description of Artificial Sequence Primer
aggctgatcactggctgtg

SEQ ID NO: 52 - DNA, 22 bp, Artificial Sequence
  Description of Artificial Sequence Primer
ccttattgtacaatggggcttc

SEQ ID NO: 53 - DNA, 24 bp, Artificial Sequence
  Description of Artificial Sequence Primer
cagacacaaggaactgaagtaacg

SEQ ID NO: 54 - DNA, 22 bp, Artificial Sequence
  Description of Artificial Sequence Primer
cactgccaagatagacaagcag

SEQ ID NO: 55 - DNA, 22 bp, Artificial Sequence
  Description of Artificial Sequence Primer
gctcttatccaggaagtccatg

SEQ ID NO: 56 - DNA, 26 bp, Artificial Sequence
  Description of Artificial Sequence Primer
tacaggatctcagacatatctccatg

SEQ ID NO: 57 - DNA, 26 bp, Artificial Sequence
  Description of Artificial Sequence Primer
aaatgtcttcccactgcataatagtc

SEQ ID NO: 58 - DNA, 26 bp, Artificial Sequence
  Description of Artificial Sequence Primer
taaggaaacaattcagtcacataagg

SEQ ID NO: 59 - DNA, 26 bp, Artificial Sequence
  Description of Artificial Sequence Primer
ctgtagcttagcaatttgttcttctg

SEQ ID NO: 60 - DNA, 25 bp, Artificial Sequence
  Description of Artificial Sequence Primer
ttatgaggcttagaatttcaaccac

SEQ ID NO: 61 - DNA, 25 bp, Artificial Sequence
  Description of Artificial Sequence Primer
aaaggctttcaaaacatttttcaac

SEQ ID NO: 62 - DNA, 27 bp, Artificial Sequence
  Description of Artificial Sequence Primer
gtagagatcagagagttgtgacatctg

SEQ ID NO: 63 - DNA, 27 bp, Artificial Sequence
  Description of Artificial Sequence Primer
tattacttttcactgttacactgctgg

SEQ ID NO: 64 - DNA, 24 bp, Artificial Sequence
  Description of Artificial Sequence Primer
gccacagagaatgaaccacttaac

SEQ ID NO: 65 - DNA, 23 bp, Artificial Sequence
  Description of Artificial Sequence Primer
gagggactatcagttgctgtttg

SEQ ID NO: 66 - DNA, 26 bp, Artificial Sequence
  Description of Artificial Sequence Primer
gcatctaatagaacgctactaccacc

SEQ ID NO: 67 - DNA, 25 bp, Artificial Sequence
  Description of Artificial Sequence Primer
ctgtgagctatcacctatccttgag

SEQ ID NO: 68 - DNA, 26 bp, Artificial Sequence
  Description of Artificial Sequence Primer
gtgacagtgaccacagaaattccccc

SEQ ID NO: 69 - DNA, 24 bp, Artificial Sequence
  Description of Artificial Sequence Primer
cacgtttctcagagctgctgctcc

SEQ ID NO: 70 - DNA, 30 bp, Artificial Sequence
  Description of Artificial Sequence Primer
gctgcacagaaaaccttacttgtttccacc

SEQ ID NO: 71 - DNA, 28 bp, Artificial Sequence
  Description of Artificial Sequence Primer
ctcgtaaatgcagaaatctccaatgccc

SEQ ID NO: 72 - DNA, 26 bp, Artificial Sequence
  Description of Artificial Sequence Primer
tccacagcctattggctcacttggac

SEQ ID NO: 73 - DNA, 30 bp, Artificial Sequence
  Description of Artificial Sequence Primer
gccctttagtgtgtctgtaattggaatcag

SEQ ID NO: 74 - DNA, 24 bp, Artificial Sequence
  Description of Artificial Sequence Primer
gcacacactgctcctccacctgac

SEQ ID NO: 75 - DNA, 27 bp, Artificial Sequence
  Description of Artificial Sequence Primer
gctgctgctgtttacagaaaggctcac

SEQ ID NO: 76 - DNA, 29 bp, Artificial Sequence
  Description of Artificial Sequence Primer
ggaaagttatccacagctactgaggaccc

SEQ ID NO: 77 - DNA, 29 bp, Artificial Sequence
  Description of Artificial Sequence Primer
tcccctccatttaatctccaaattcaccc

SEQ ID NO: 78 - DNA, 23 bp, Artificial Sequence
  Description of Artificial Sequence Primer
ctcagcatttgccgccgtaactt

SEQ ID NO: 79 - DNA, 24 bp, Artificial Sequence
  Description of Artificial Sequence Primer
gaaaactacaaatcccaggagcac

SEQ ID NO: 80 - DNA, 24 bp, Artificial Sequence
  Description of Artificial Sequence Primer
ctcacgaaatatgagcttcaccac

SEQ ID NO: 81 - DNA, 27 bp, Artificial Sequence
  Description of Artificial Sequence Primer
ttactgatcgtctgctccctagagtcc

SEQ ID NO: 82 - DNA, 27 bp, Artificial Sequence
  Description of Artificial Sequence Primer
atcttctgctcagtcagaatcccatgc

SEQ ID NO: 83 - DNA, 23 bp, Artificial Sequence
  Description of Artificial Sequence Primer
tgttgtagatcacttcaaggtgc

SEQ ID NO: 84 - DNA, 26 bp, Artificial Sequence
  Description of Artificial Sequence Primer
ccatatccaaattcccttggtgtgag

SEQ ID NO: 85 - DNA, 20 bp, Artificial Sequence
  Description of Artificial Sequence Primer
tgagaatgaggtgggggtgg

SEQ ID NO: 86 - DNA, 19 bp, Artificial Sequence
  Description of Artificial Sequence Primer
tggggaaccggatcaggac

SEQ ID NO: 87 - DNA, 22 bp, Artificial Sequence
  Description of Artificial Sequence Primer
gcatcctaccaccaactcgtcc

SEQ ID NO: 88 - DNA, 23 bp, Artificial Sequence
  Description of Artificial Sequence Primer
agttctgagaccgttcttccacc

SEQ ID NO: 89 - DNA, 23 bp, Artificial Sequence
  Description of Artificial Sequence Primer
gctgcggacataaatcttaaagc

SEQ ID NO: 90 - DNA, 20 bp, Artificial Sequence
  Description of Artificial Sequence Primer
agggtctcactctgattgcc

SEQ ID NO: 91 - DNA, 2428 bp, Homo sapiens
gcactgaggtcaccctccaggctgtggaacctttgttctttcactctttgcaataaatcttgctgctgctcactctttgggtccacactgcctttatgagctgtaacactcactgggaatgtctgcagcttcactcctgaagccagcgagaccacgaacccaccaggaggaacaaacaactccagacgcgcagccttaagagctgtaacactcaccgcgaaggtctgcagcttcactcctgagccagccagaccacgaacccaccagaaggaagaaactccaaacacatccgaacatcagaaggagcaaactcctgacacgccacctttaagaaccgtgacactcaacgctagggtccgcggcttcattcttgaagtcagtgagaccaagaacccaccaattccggacacgctaattgttgtagatcatcacttcaaggtgcccatatctttctagtggaaaaattattctggcctccgctgcatacaaatcaggcaaccagaattctacatatataaggcaaagtaacatcctagacatggctttagagatccacatgtcagaccccatgtgcctcatcgagaactttaatgagcagctgaaggttaatcaggaagctttggagatcctgtctgccattacgcaacctgtagttgtggtagcgattgtgggcctctatcgcactggcaaatcctacctgatgaacaagctggctgggaagaacaagggcttctctgttgcatctacggtgcagtctcacaccaagggaatttggatatggtgtgtgcctcatcccaactggccaaatcacacattagttctgcttgacaccgagggcctgggagatgtagagaaggctgacaacaagaatgatatccagatctttgcactggcactcttactgagcagcacctttgtgtacaatactgtgaacaaaattgatcagggtgctatcgacctactgcacaatgtgacagaactgacagatctgctcaaggcaagaaactcacccgaccttgacagggttgaagatcctgctgactctgcgagcttcttcccagacttagtgtggactctgagagatttctgcttaggcctggaaatagatgggcaacttgtcacaccagatgaatacctggagaattccctaaggccaaagcaaggtagtgatcaaagagttcaaaatttcaatttgccccgtctgtgtatacagaagttctttccaaaaaagaaatgctttatctttgacttacctgctcaccaaaaaaagcttgcccaacttgaaacactgcctgatgatgagctagagcctgaatttgtgcaacaagtgacagaattctgttcctacatctttagccattctatgaccaagactcttccaggtggcatcatggtcaatggatctcgtctaaagaacctggtgctgacctatgtcaatgccatcagcagtggggatctgccttgcatagagaatgcagtcctggccttggctcagagagagaactcagctgcagtgcaaaaggccattgcccactatgaccagcaaatgggccagaaagtgcagctgcccatggaaaccctccaggagctgctggacctgcacaggaccagtgagagggaggccattgaagtcttcatgaaaaactctttcaaggatgtagaccaaagtttccagaaagaattggagactctactagatgcaaaacagaatgacatttgtaaacggaacctggaagcatcctcggattattgctcggctttacttaaggatatttttggtcctctagaagaagcagtgaagcagggaatttattctaagccaggaggccataatctcttcattcagaaaacagaagaactgaaggcaaagtactatcgggagcctcggaaaggaatacaggctgaagaagttctgcagaaatatttaaagtccaaggagtctgtgagtcatgcaatattacagactgaccaggctctcacagagacggaaaaaaagaagaaagaggcacaagtgaaagcagaagctgaaaaggctgaagcgcaaaggttggcggcgattcaaaggcagaacgagcaaatgatgcaggagagggagagactccatcaggaacaagtgagacaaatggagatagccaaacaaaattggctggcagagcaacagaaaatgcaggaacaacagatgcaggaacaggctgcacagctcagcacaacattccaagctcaaaatagaagccttctcagtgagctccagcacgcccagaggactgttaataacgatgatccatgtgttttactctaaagtgctaaatatgggagtttcctttttttactctttgtcactgatgacacaacagaaaagaaactgtagaccttgggacaatcaacatttaaataaactttataattattttttcaaactttcaaaaaa

SEQ ID NO: 92 - protein, 586 aa, Homo sapiens
MALEIHMSDPMCLIENFNEQLKVNQEALEILSAITQPVVVVAIVGLYRTGKSYLMNKLAGKNKGFSVASTVQSHTKGIWIWCVPHPNWPNHTLVLLDTEGLGDVEKADNKNDIQIFALALLLSSTFVYNTVNKIDQGAIDLLHNVTELTDLLKARNSPDLDRVEDPADSASFFPDLVWTLRDFCLGLEIDGQLVTPDEYLENSLRPKQGSDQRVQNFNLPRLCIQKFFPKKKCFIFDLPAHQKKLAQLETLPDDELEPEFVQQVTEFCSYIFSHSMTKTLPGGIMVNGSRLKNLVLTYVNAISSGDLPCIENAVLALAQRENSAAVQKAIAHYDQQMGQKVQLPMETLQELLDLHRTSEREAIEVFMKNSFKDVDQSFQKELETLLDAKQNDICKRNLEASSDYCSALLKDIFGPLEEAVKQGIYSKPGGHNLFIQKTEELKAKYYREPRKGIQAEEVLQKYLKSKESVSHAILQTDQALTETEKKKKEAQVKAEAEKAEAQRLAAIQRQNEQMMQERERLHQEQVRQMEIAKQNWLAEQQKMQEQQMQEQAAQLSTTFQAQNRSLLSELQHAQRTVNNDDPCVLL

SEQ ID NO: 93 - DNA, 26 bp, Artificial Sequence
  Description of Artificial Sequence Primer
agaaggaagaaactccaaacacatcc

(19) United States
(12) Patent Application Publication
(10) Pub. No.: US 2004/0197782 A1
(43) Pub. Date: Oct. 7, 2004

(54) Novel marker for the diagnosis and therapy of tumours

(76) Inventors: Stefan Elchmuller, Edingen-Neckarhausen (DE); Dirk Schadendorf, Mannheim (DE); Dirk Usener, Wiesbaden (DE)

Correspondence Address: HELLER EHRMAN WHITE & MCAULIFFE LLP, 1666 K STREET,NW, SUITE 300, WASHINGTON, DC, 20006, US

(21) Appl. No.: 10/416,330

(22) PCT Filed: Nov. 8, 2001
(86) PCT No.: PCT/DE01/04229

(30) Foreign Application Priority Data
Nov. 8, 2000 (DE) 100 55 285.4

(51) Int. Cl. C12Q 1/68

(52) U.S. Cl. 435/6

(57) ABSTRACT

The present invention relates to novel markers for tumors, preferably CTCL. The present invention also relates to the use thereof for the diagnosis and/or therapy of tumoral diseases, preferably CTCL.

The present invention relates to the use of novel markers for the diagnosis and/or therapy of tumoral diseases, preferably CTCL.

Cutaneous T-cell lymphomas (CTCL) represent a heterogenous group of diseases in which CD4 T-cells prevail as the malignant cell type. In most cases, the monoclonal or at least oligoclonal origin of the malignant cells is documented by means of T-cell receptor rearrangements. Along with various other subtypes, mycosis fungoides and the Sézary syndrome (SS) represent the most frequent forms of CTCL. Both diseases are monoclonal T-helper memory lymphomas which are characterized by cutaneous plaques, tumors or erythrodermia, SS being additionally characterized by a generalized lymphadenopathy and the presence of neoplastic T-cells in the peripheral blood.

The therapeutic approaches comprise the stage-dependent selection of PUVA (psoralen and UV-A), retinoids, interferon α-2a in combination with acitretin or PUVA, various immunomodulators, electron irradiation or extracorporeal photopheresis. These methods are successful in the early disease stages but not in the aggressive subsequent stages. Immunological therapies, e.g. vaccination with peptides or peptide-loaded dendritic cells as used already for treating melanomas, are counted among the possibly useful future therapies for CTCL.

The presence and activity of CD8⁺ cells in the case of CTCL was correlated with the prognosis. It was possible to show that CD8⁺-reactive infiltrates are CTCL-specific and lytic. Thus, although immunotherapies might represent a promising conception for treating CTCL, a precondition for such a strategy is the identification of tumor-specific antigens. In this connection, the T-cell receptor as such was proposed as an antigen (similar to the idiotype-immunoglobulins as a target for B-cell-specific T-cells). However, in both cases there is the drawback that the antigen T-cell receptor of each individual patient should be identified. In summary, it should, however, be pointed out that no tumor-associated antigens are currently known for tumor kinds, such as CTCL, and therefore the chances of a specific diagnosis and/or therapy are greatly limited.

The present invention is thus substantially based on the technical problem of identifying and providing markers (genes and/or their products) which are correlated with tumors, in particular CTCL, and are optionally of diagnostic use and/or, based on a vaccination therapy, of therapeutic use.

The solution to this technical problem was achieved by providing the embodiments characterized in the claims.

Surprisingly, a number of genes were found the expression of which is correlated with CTCL. In the experiments resulting in the present invention, CTCL-specific antigens were identified by screening a testis cDNA library and/or a cDNA library established from tumor RNA of different cutaneous lymphomas, with serums from tumor patients. About 3×10⁶ recombinants were screened with serums from patients suffering from Sézary syndrome or Mycosis fungoides. The results show that tumor antigens from CTCL tumors can be identified using antibodies derived from tumor patients. It was possible to identify positive clones belonging to 19 different genes/ORFs which also comprised five formerly unknown sequences. All of the tumor antigens found are specific, i.e. only tumor patients but no healthy persons produce antibodies directed thereagainst although 13 of these tumor antigens are expressed in at least 21 % of the tested control tissues. Moreover, a tumor-specific antigen was found which is only expressed in testis and tumor tissues. This antigen is se2-1, which was found in a CTCL tumor. This gene shows some similarity with SCP-1, a protein correlated with mitosis. Four serums from CTCL patients reacted with different SCP-1-similar clones. Thus, it was possible by means of the experiments leading to the present invention to identify CTCL-associated antigens for the first time (irrespective of the T-cell receptor) which are thus valuable tumor markers. The identification of such antigens is of interest since the coded proteins and peptides derived therefrom serve as target structures, e.g. for cytotoxic cells, and can be used as antigens for the production of diagnostic or therapeutic antibodies. For tumor therapy, the peptides encoded by the nucleic acids according to the invention and/or fragments thereof can be applied either directly or be loaded onto antigen-presenting cells. The peptides representing antigens can also be expressed in different cells (e.g. dendritic cells as antigen-presenting cells) by means of vectors. Furthermore, the identified nucleic acids serve as a basis for developing diagnostic tests to ensure a more reliable and early diagnosis in affected persons in future. Moreover, functional analyses of the proteins will no doubt contribute to an understanding of tumor development. The nucleic acids according to the invention should thus be regarded as candidate genes for studying the pathomechanisms underlying different tumoral diseases, such as CTCL.

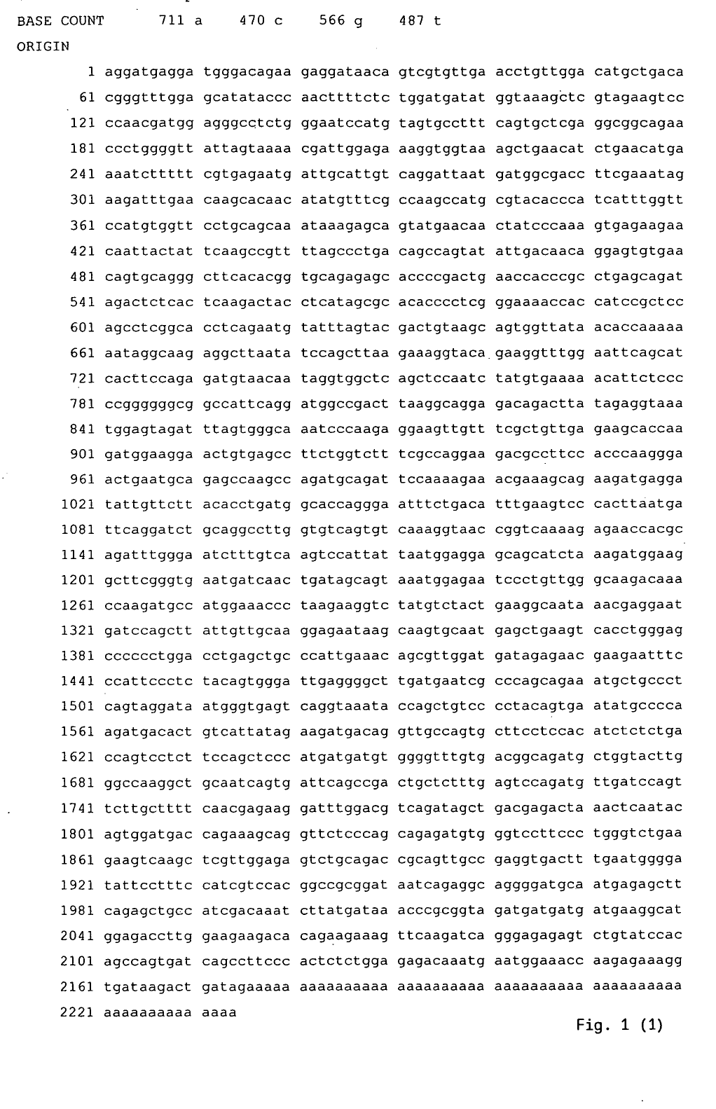

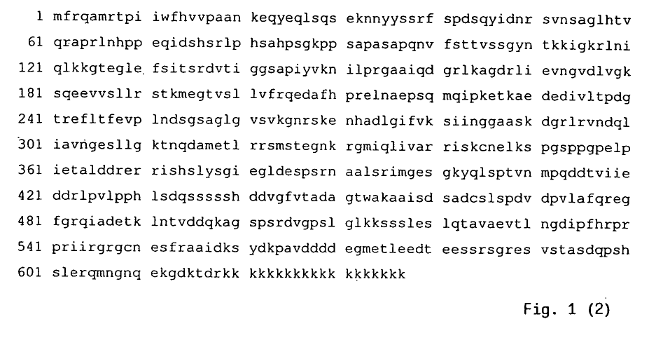

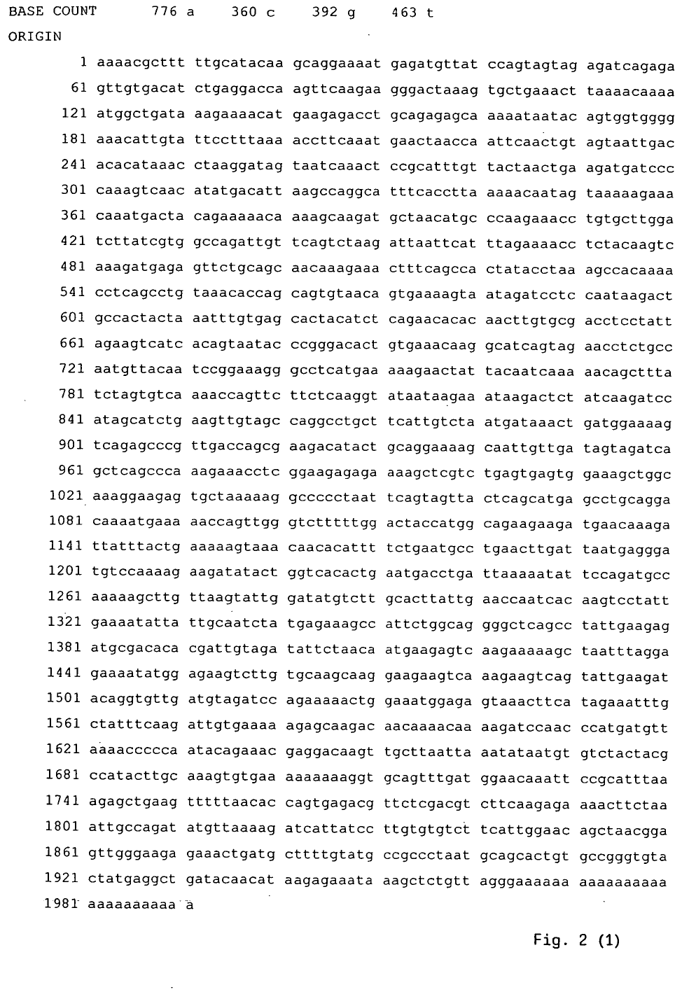

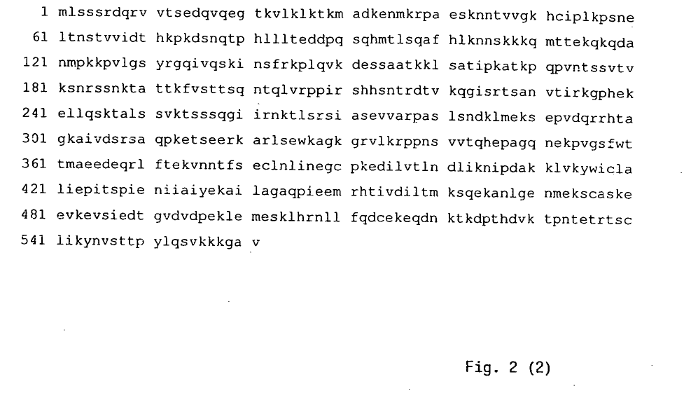

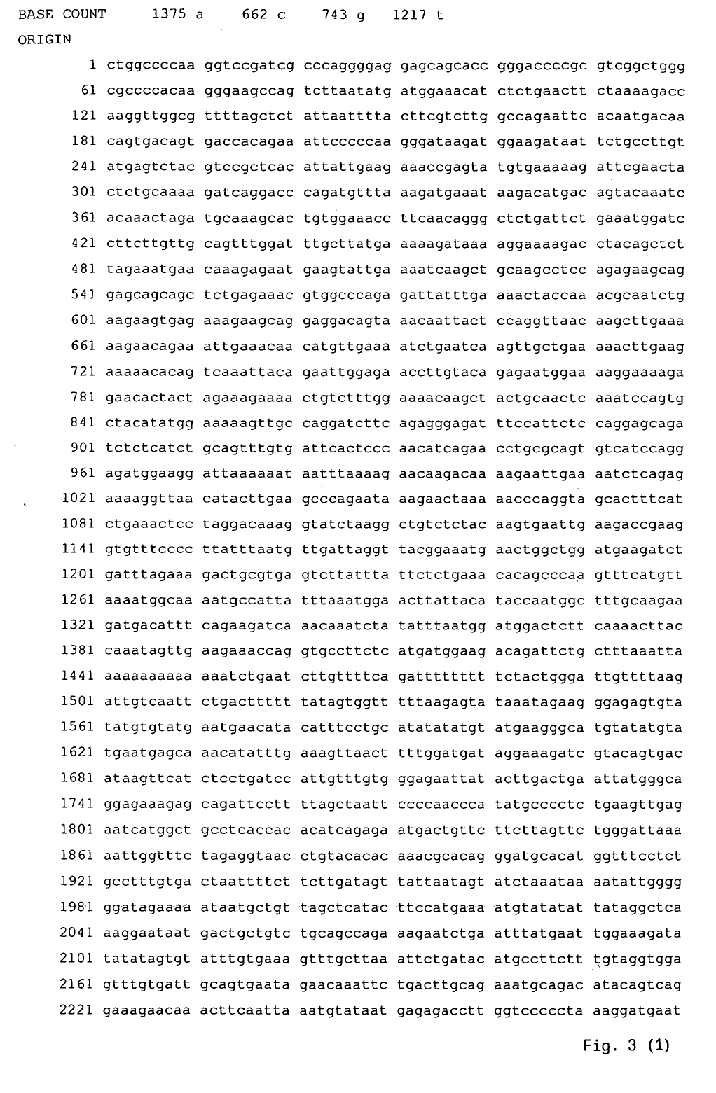

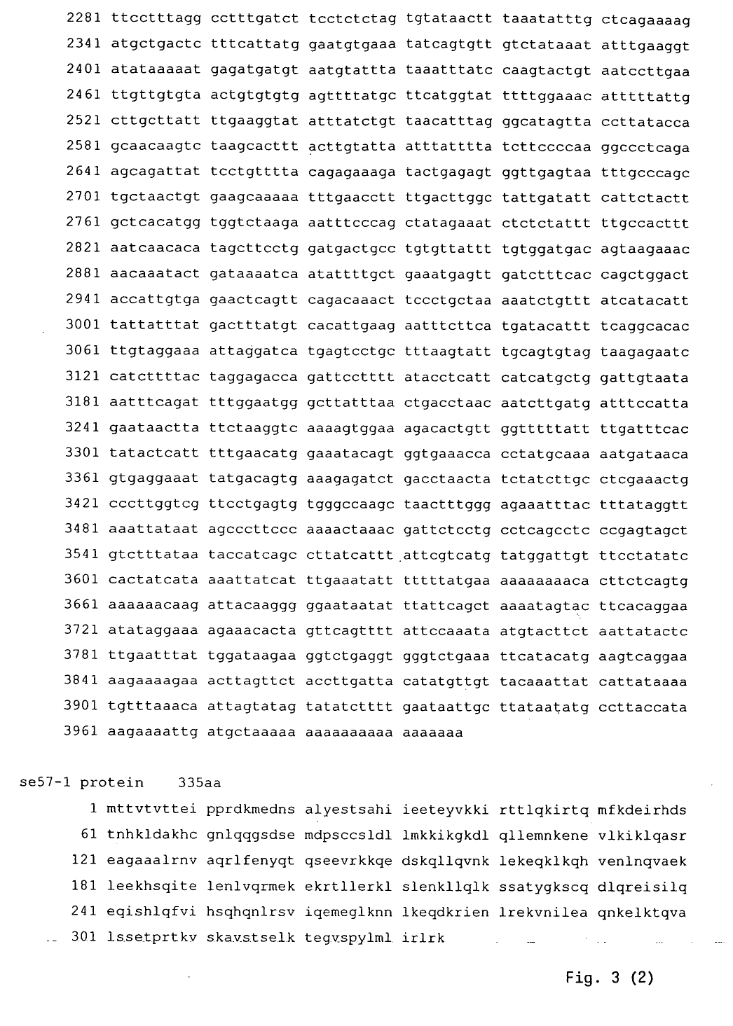

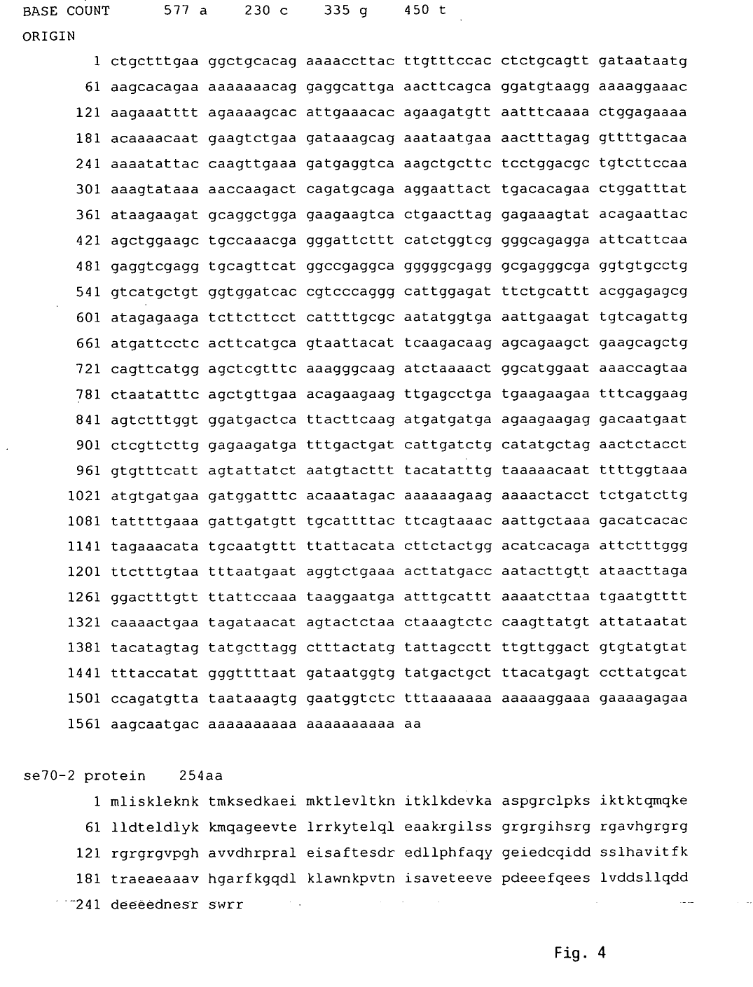

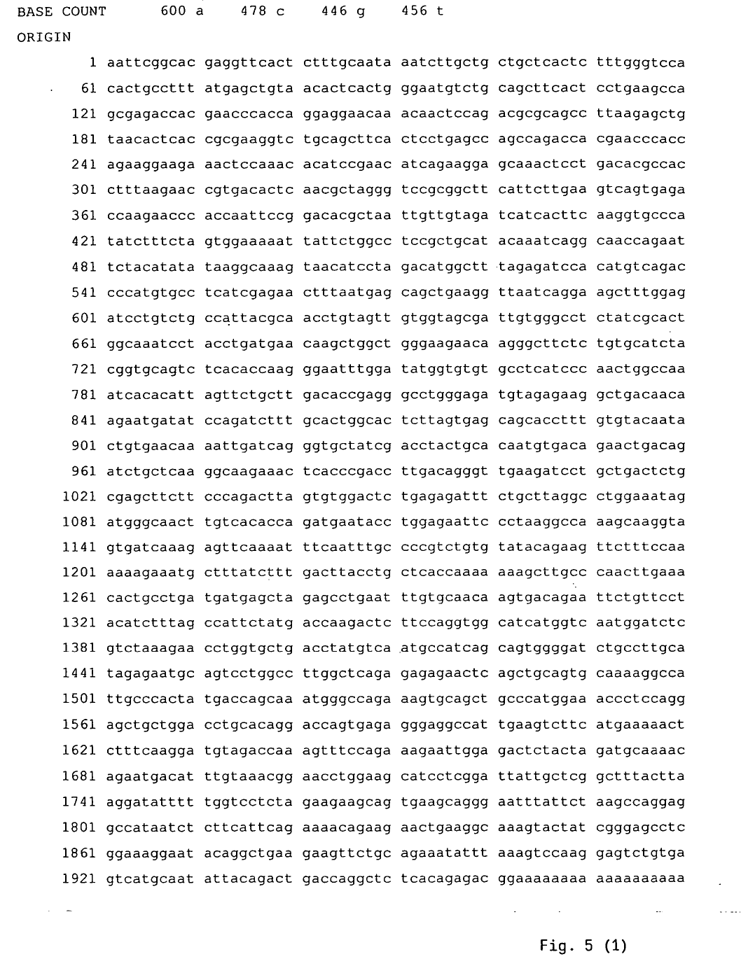

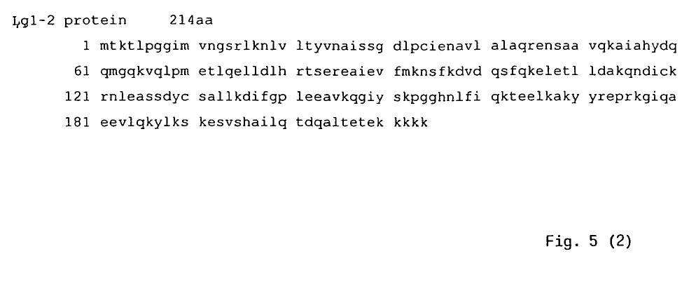

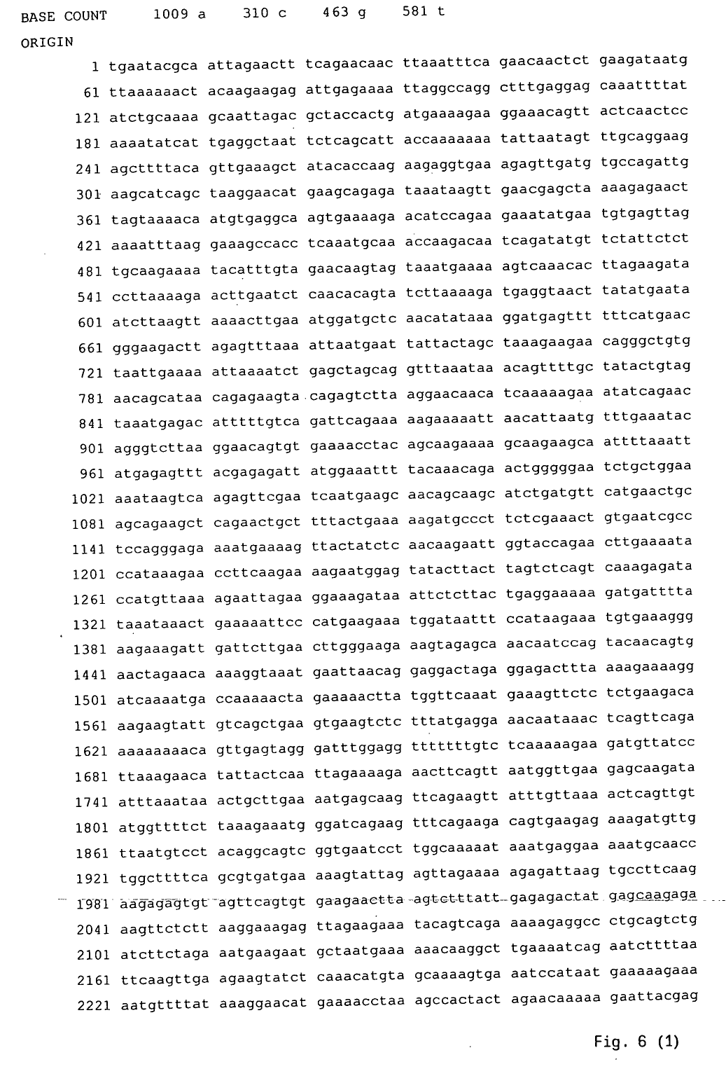

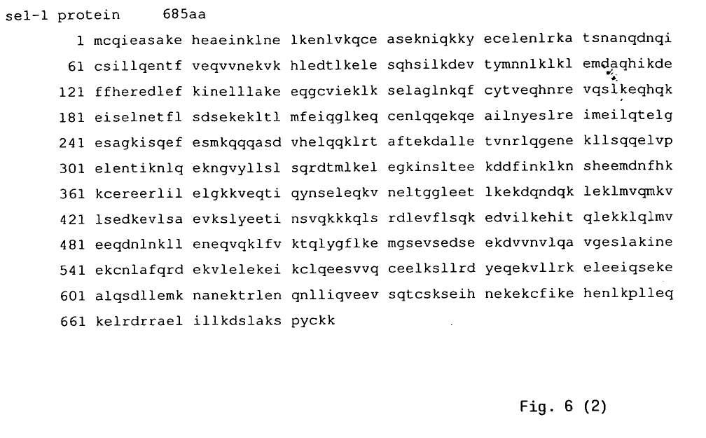

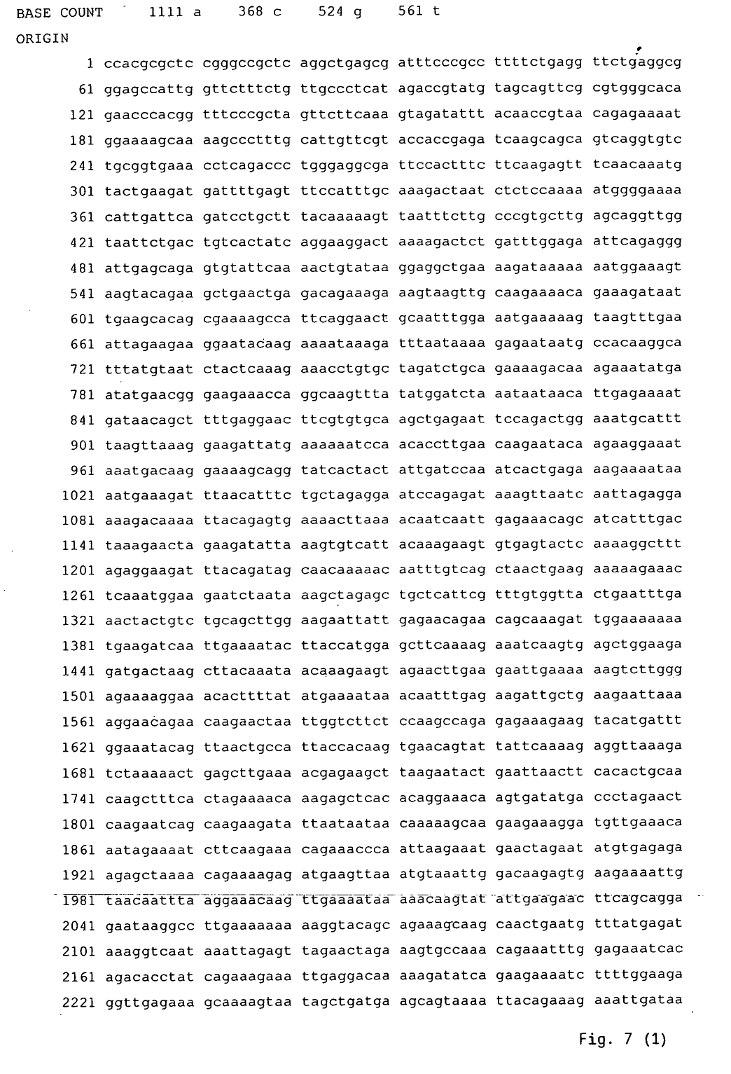

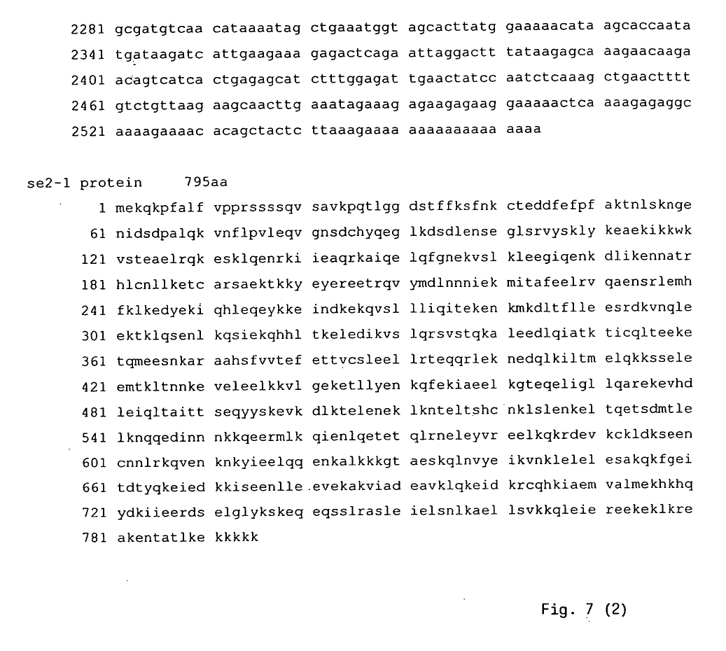

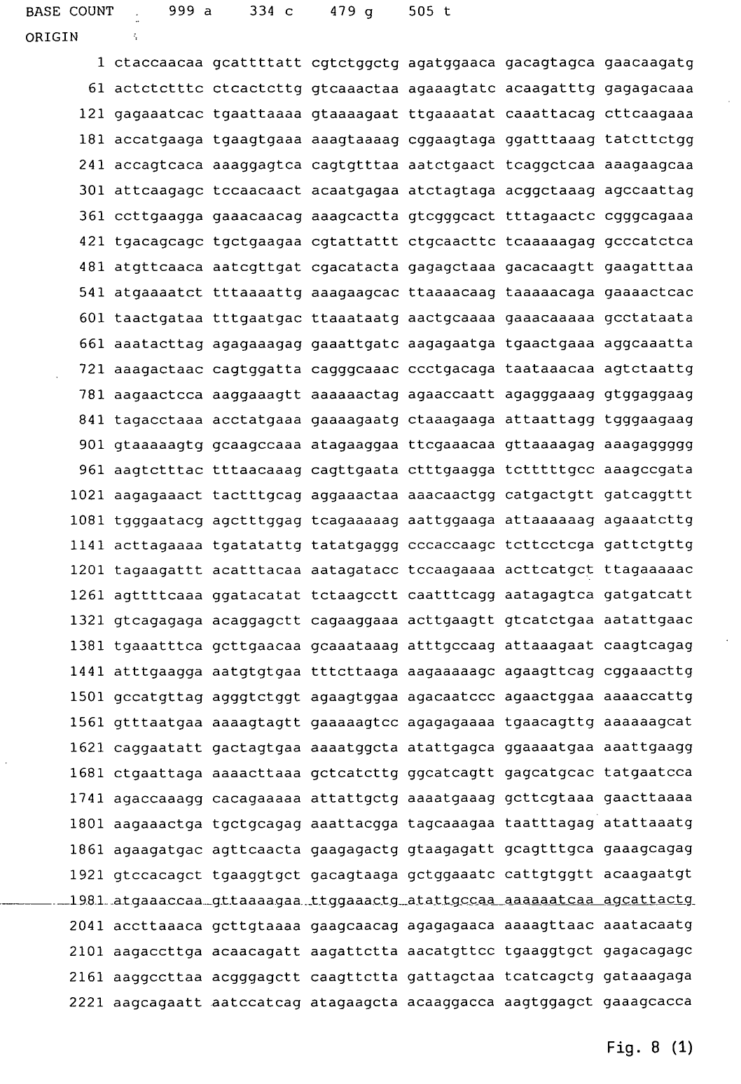

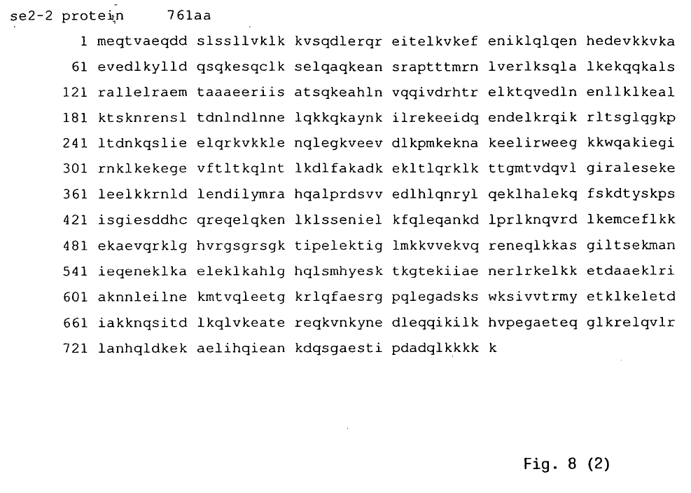

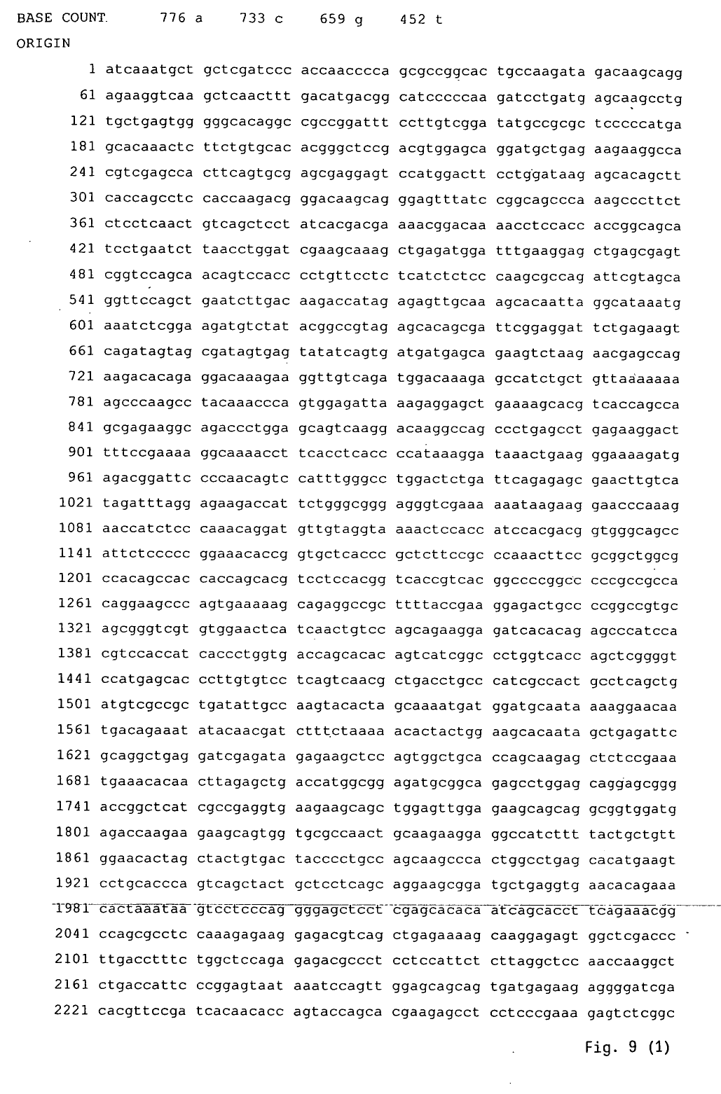

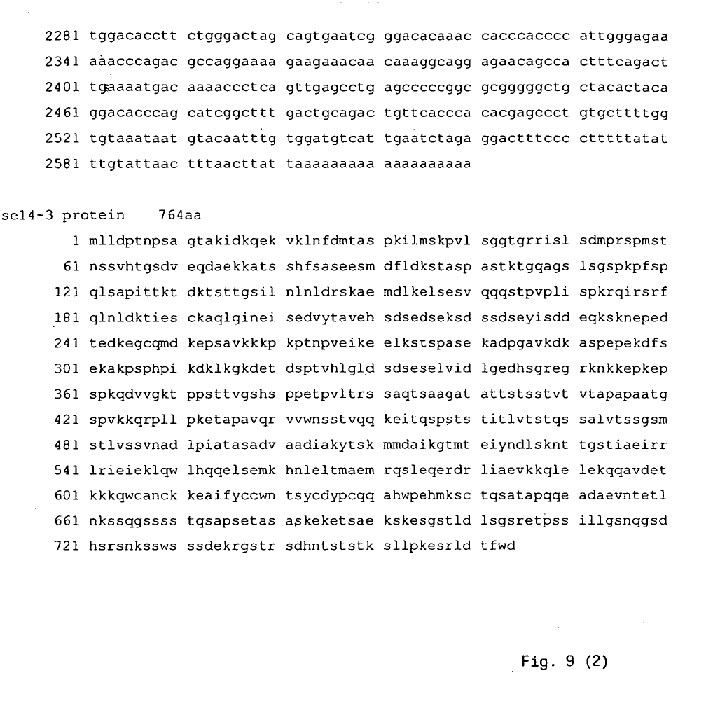

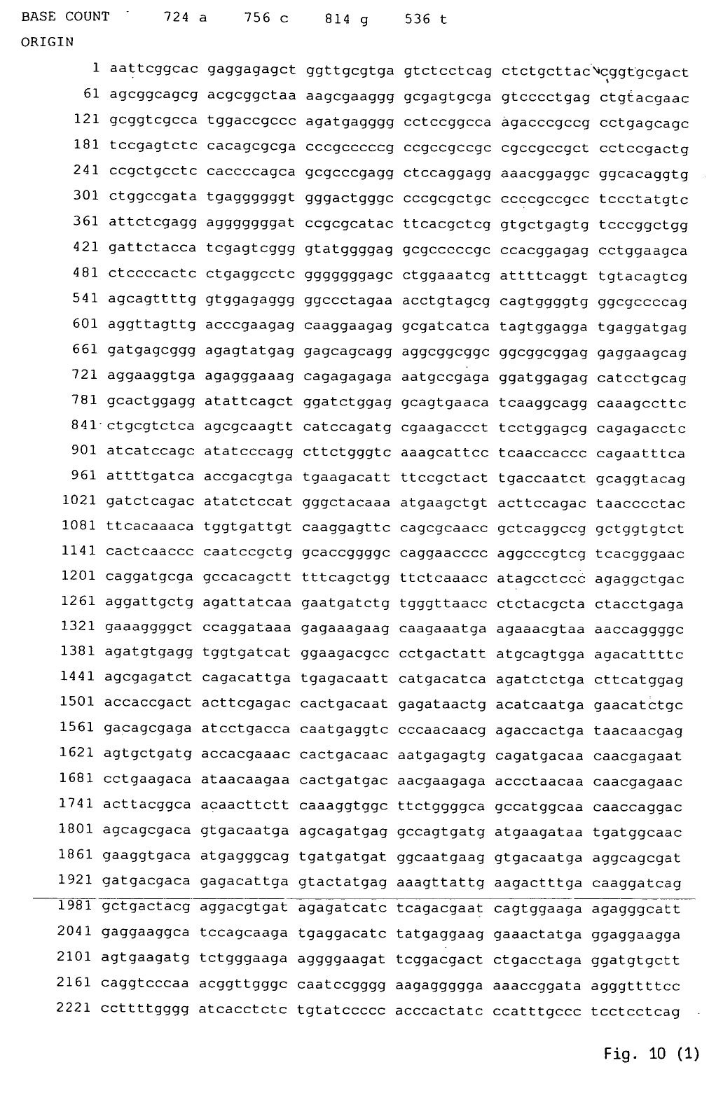

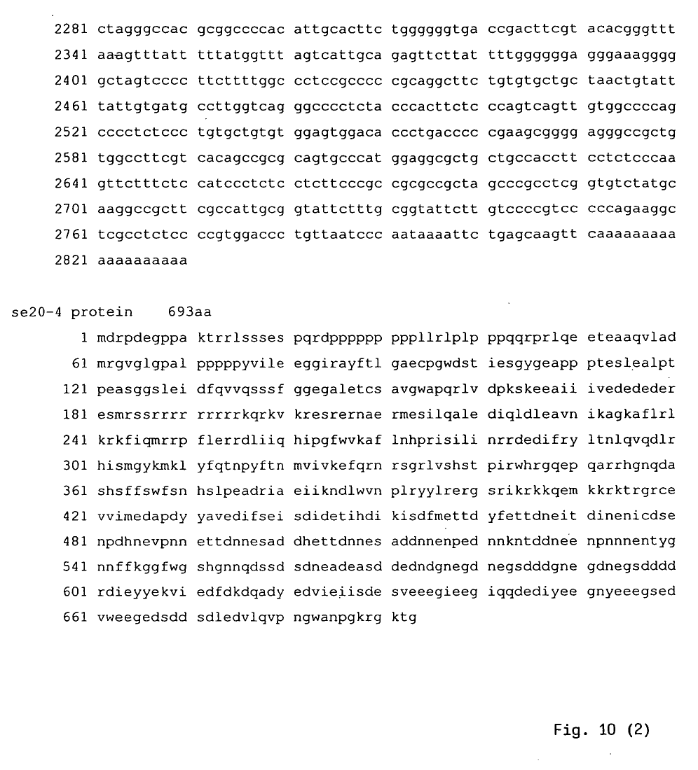

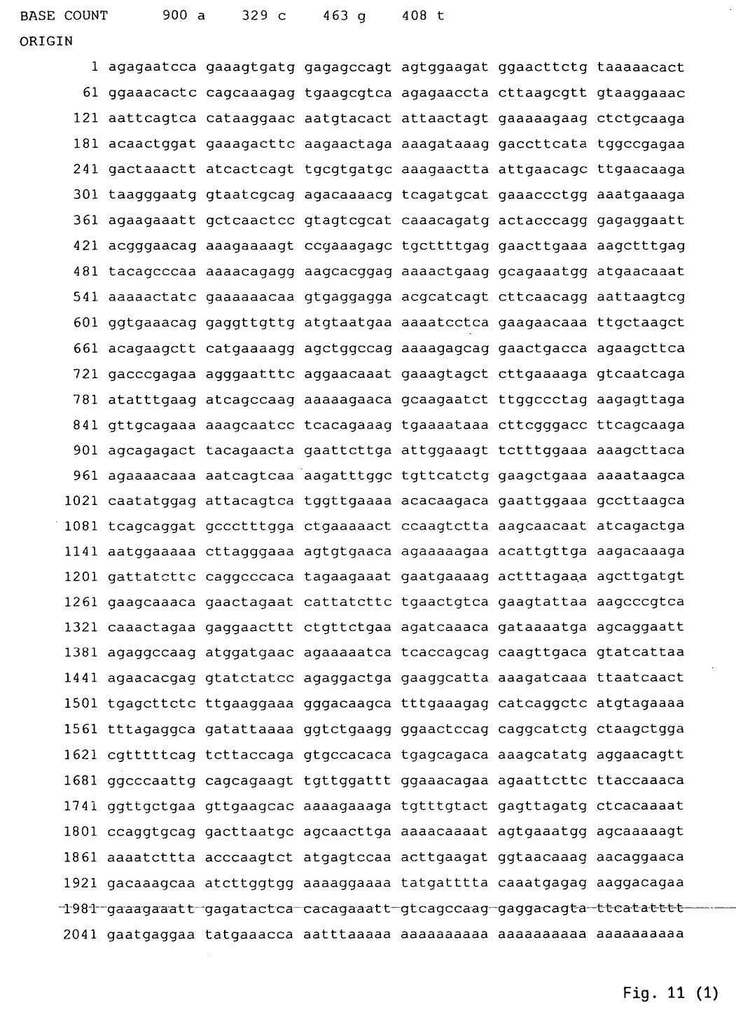

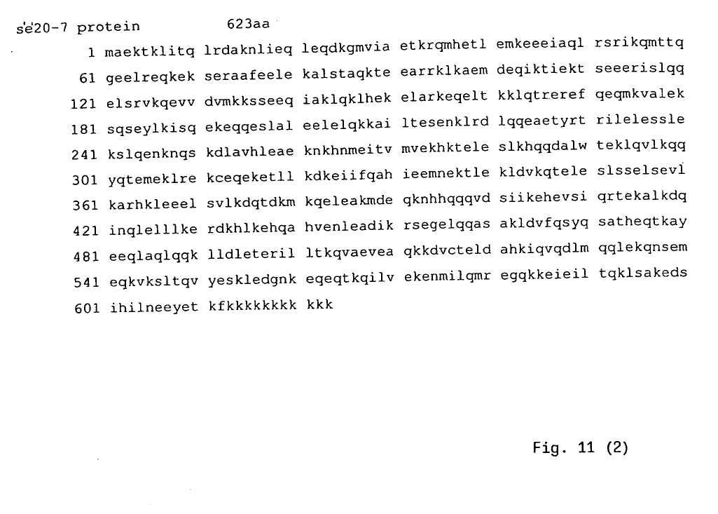

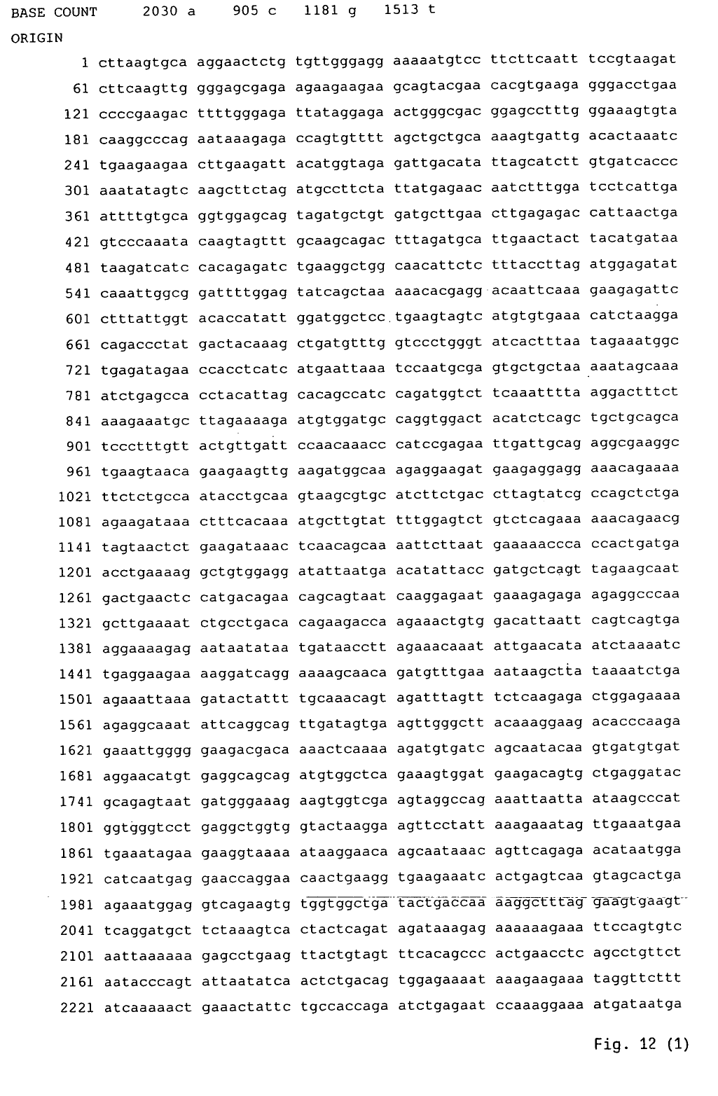

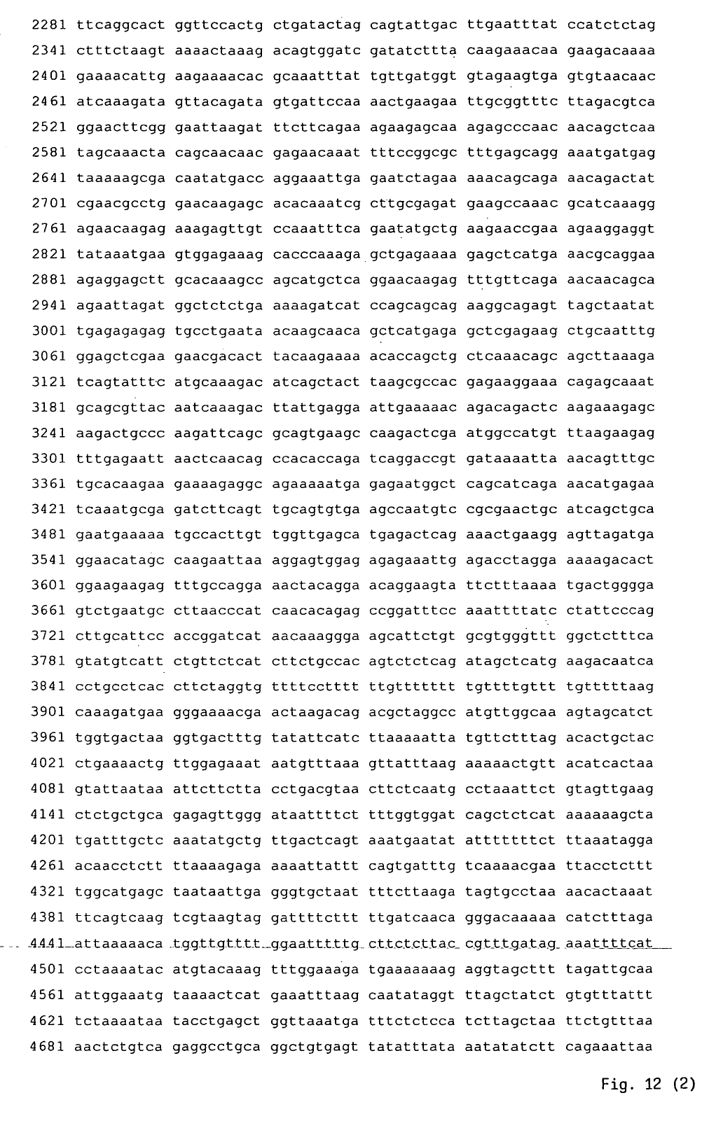

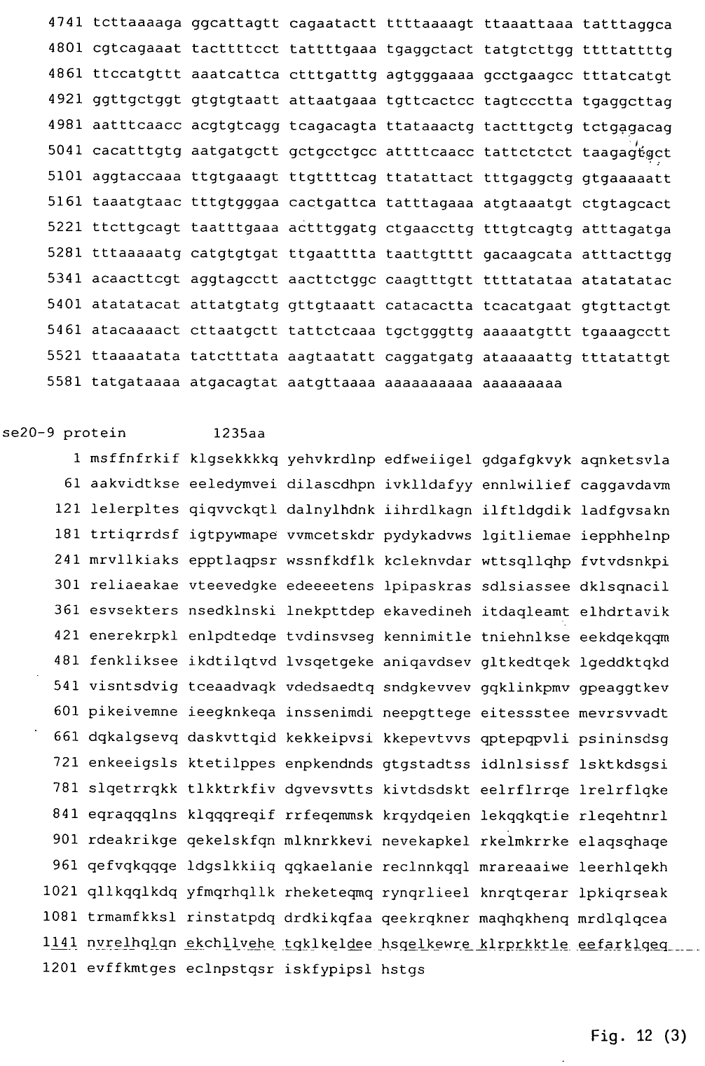

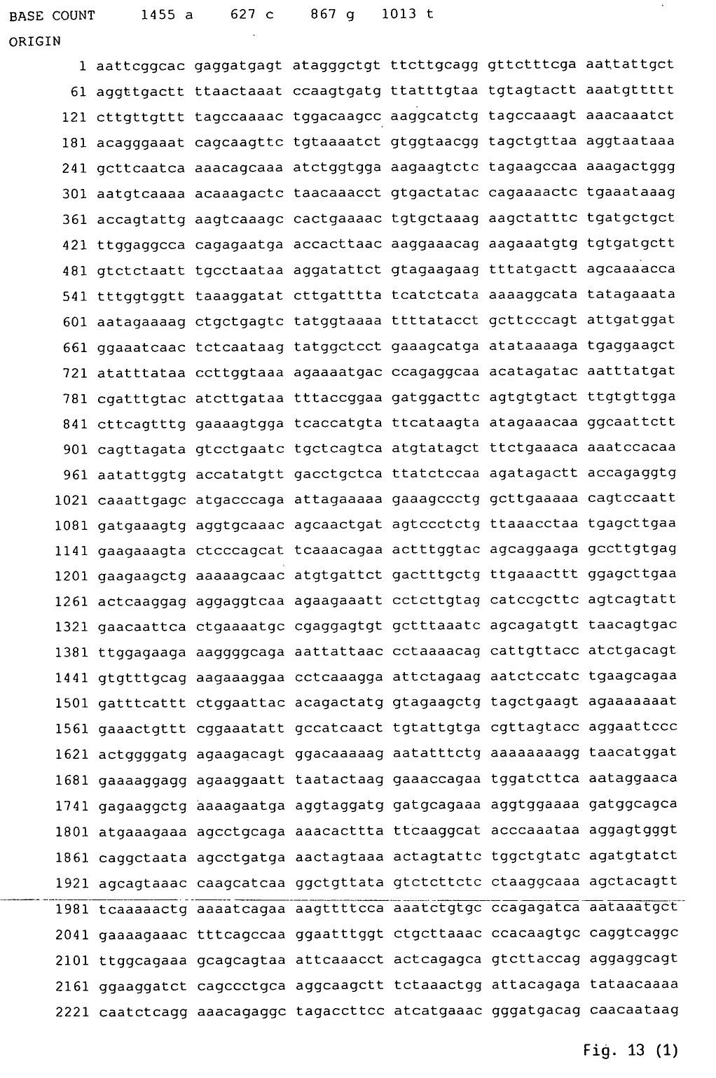

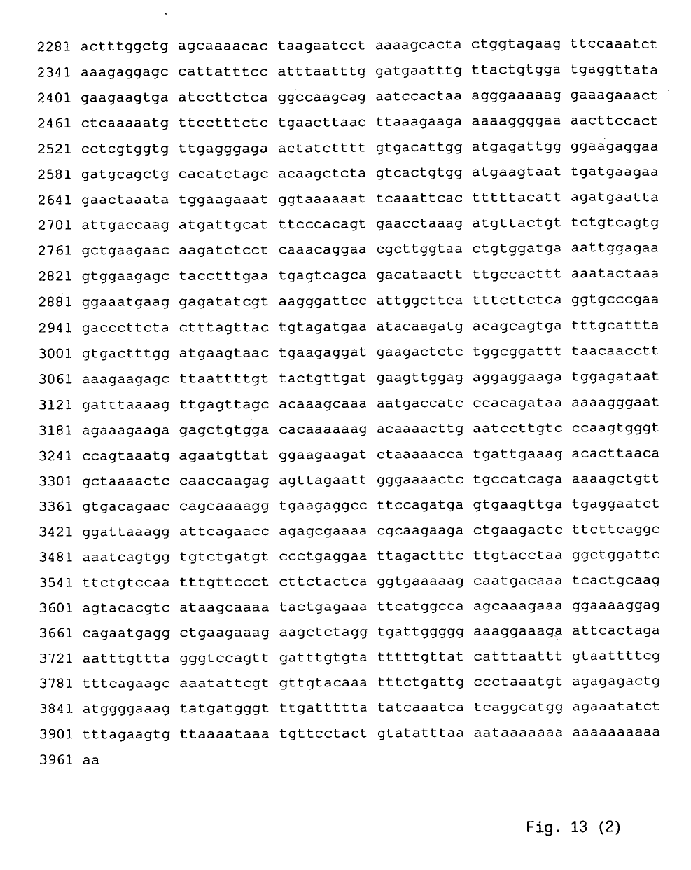

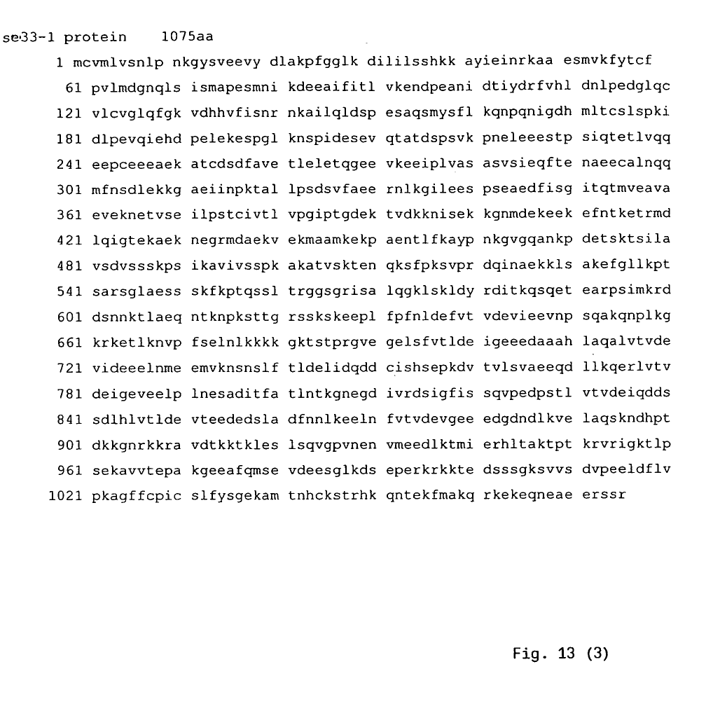

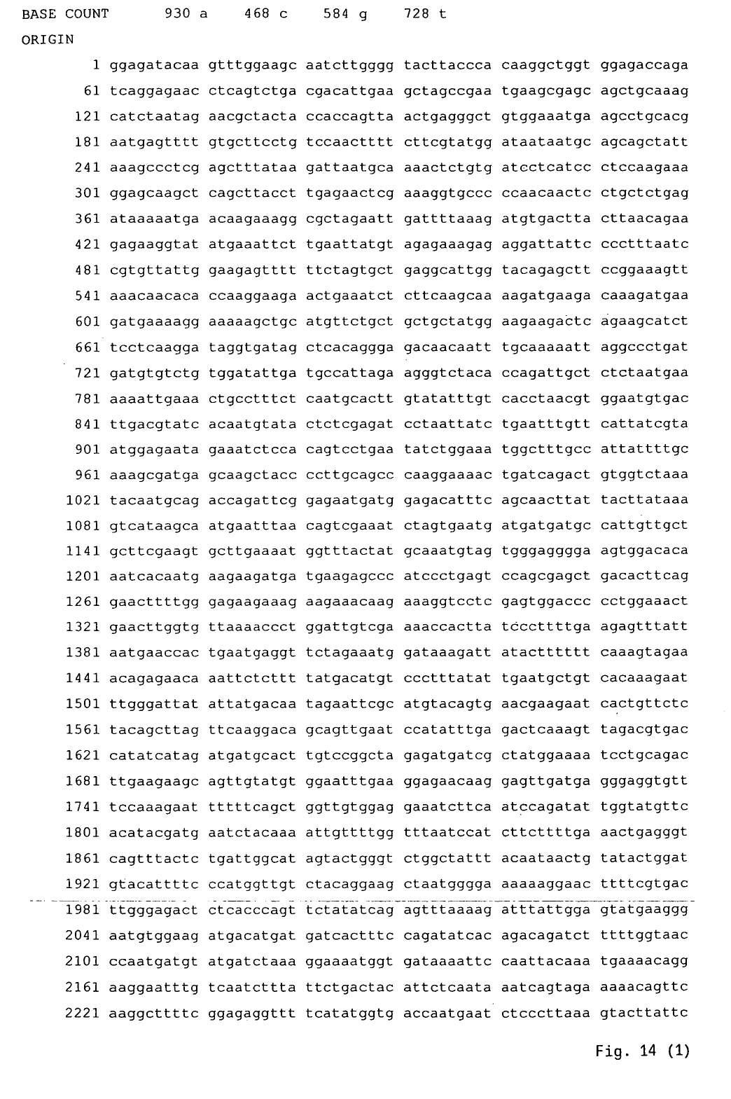

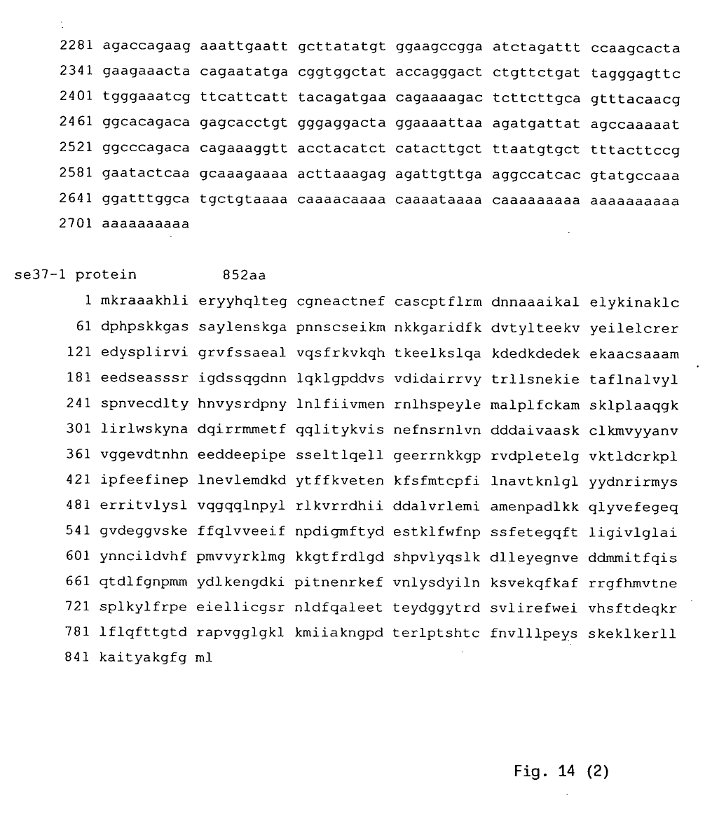

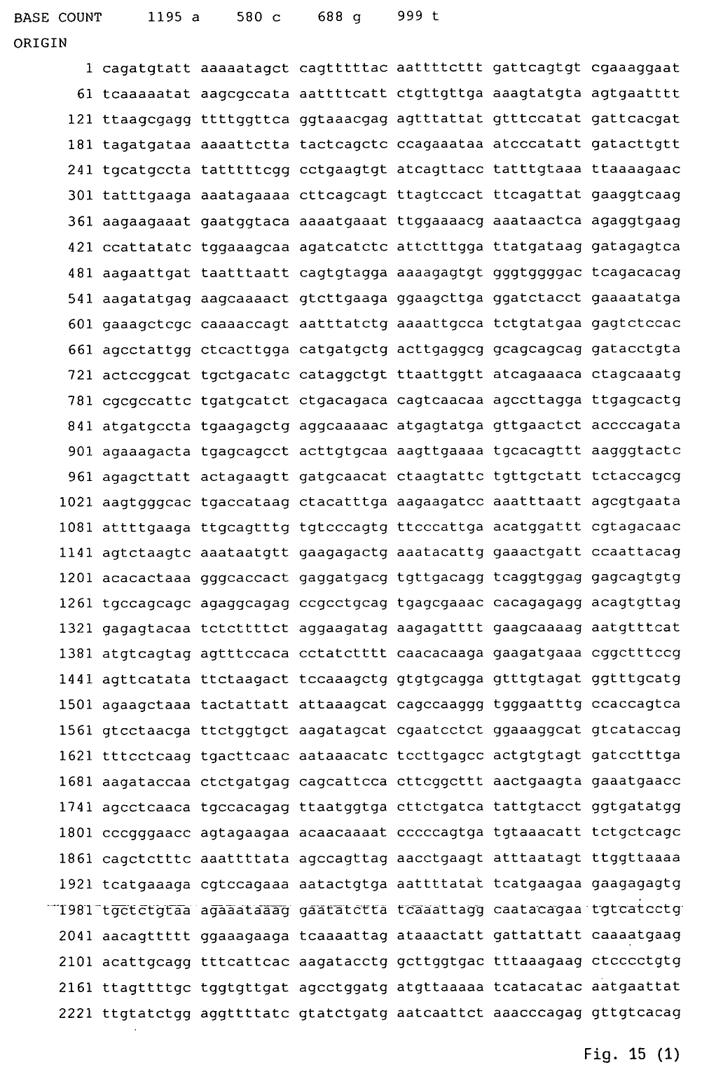

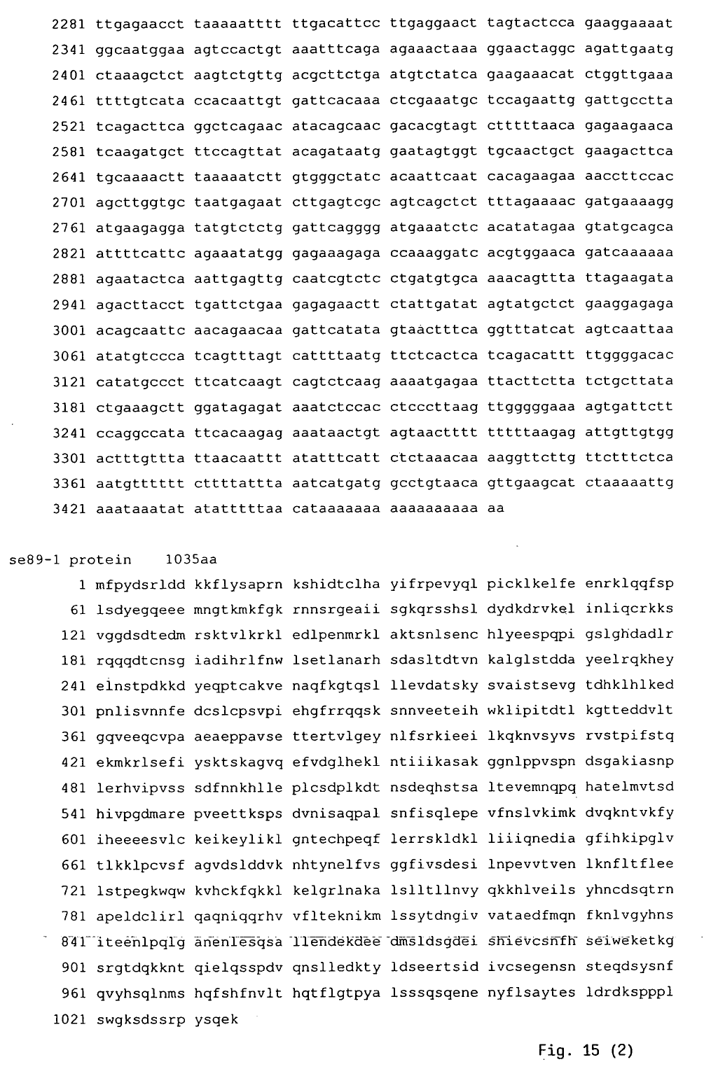

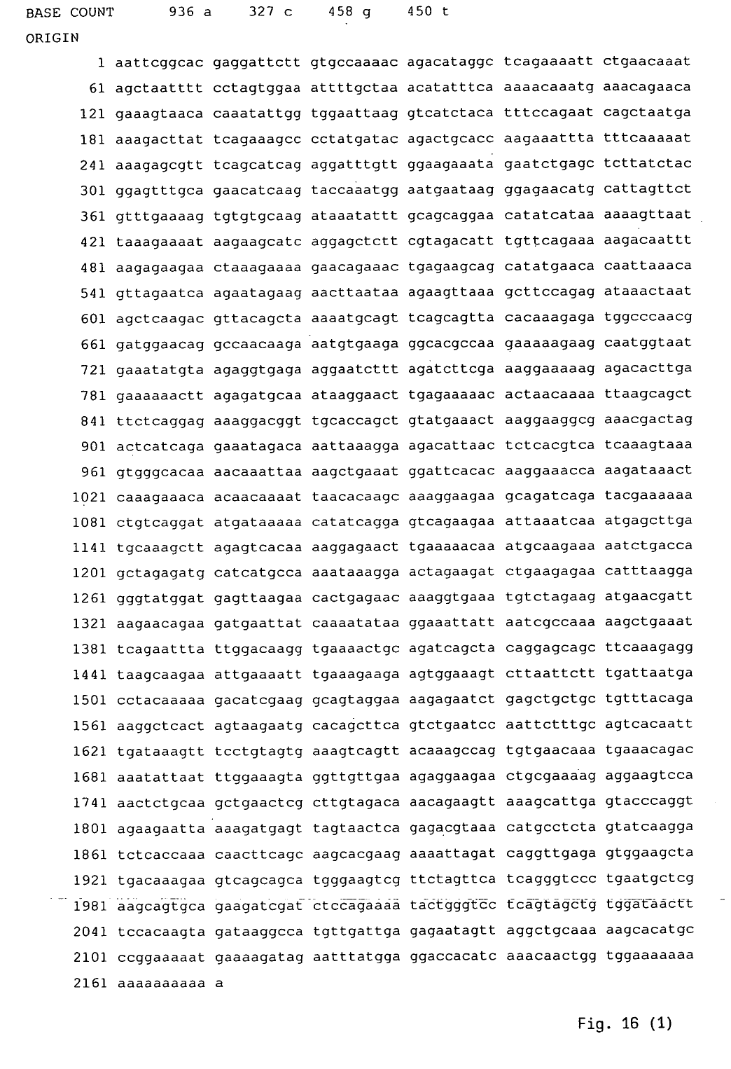

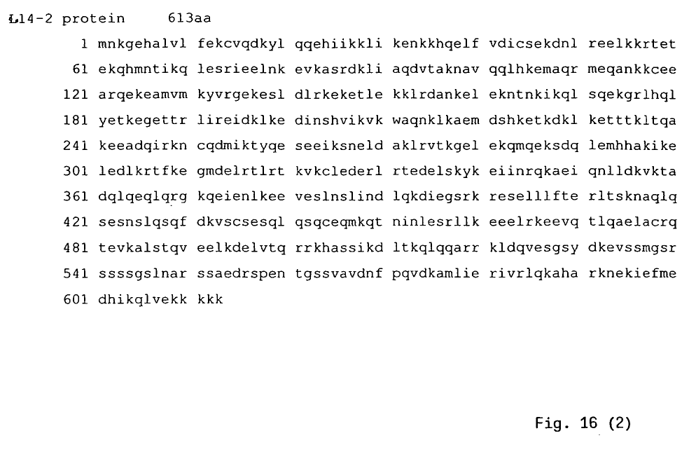

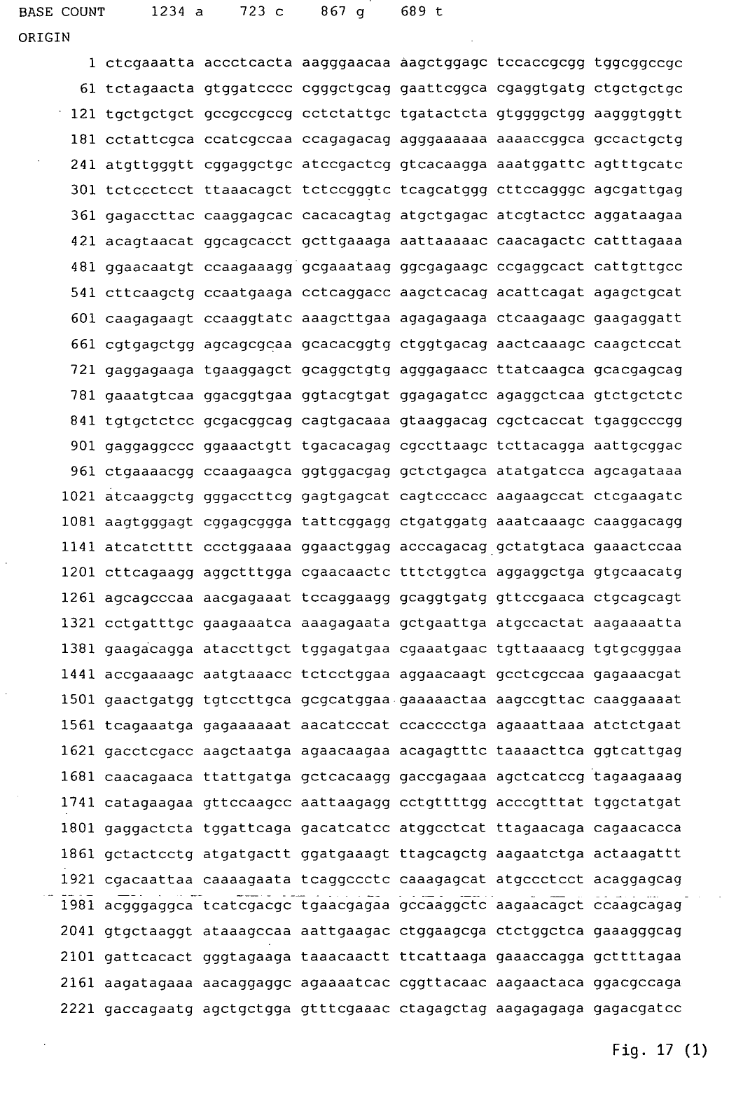

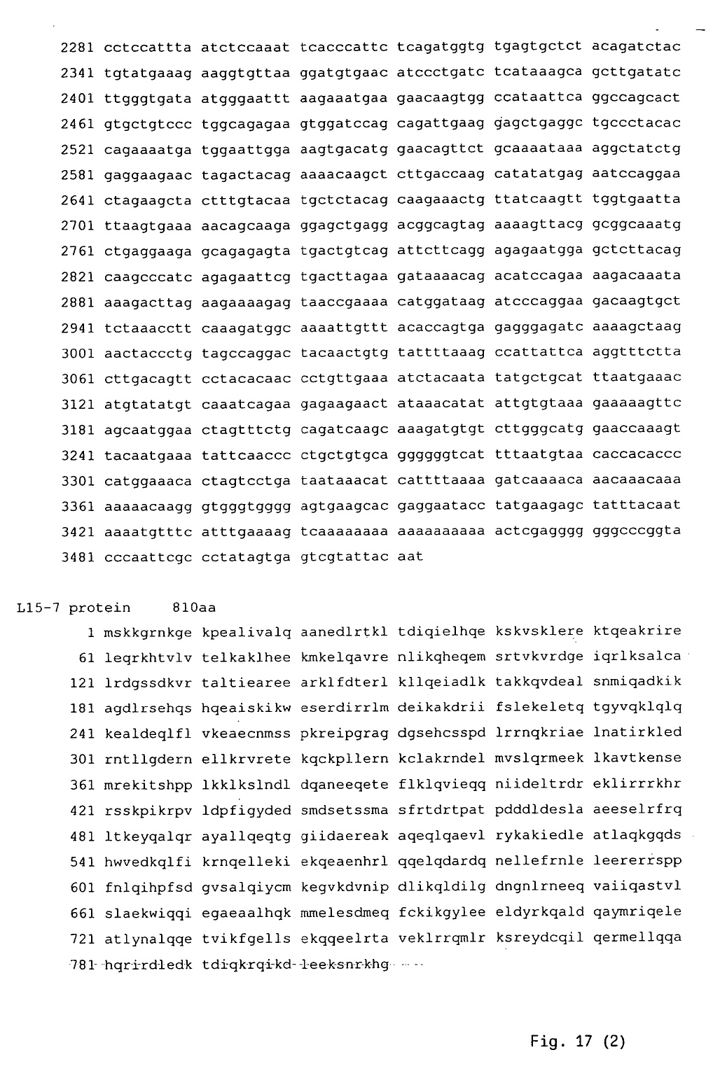

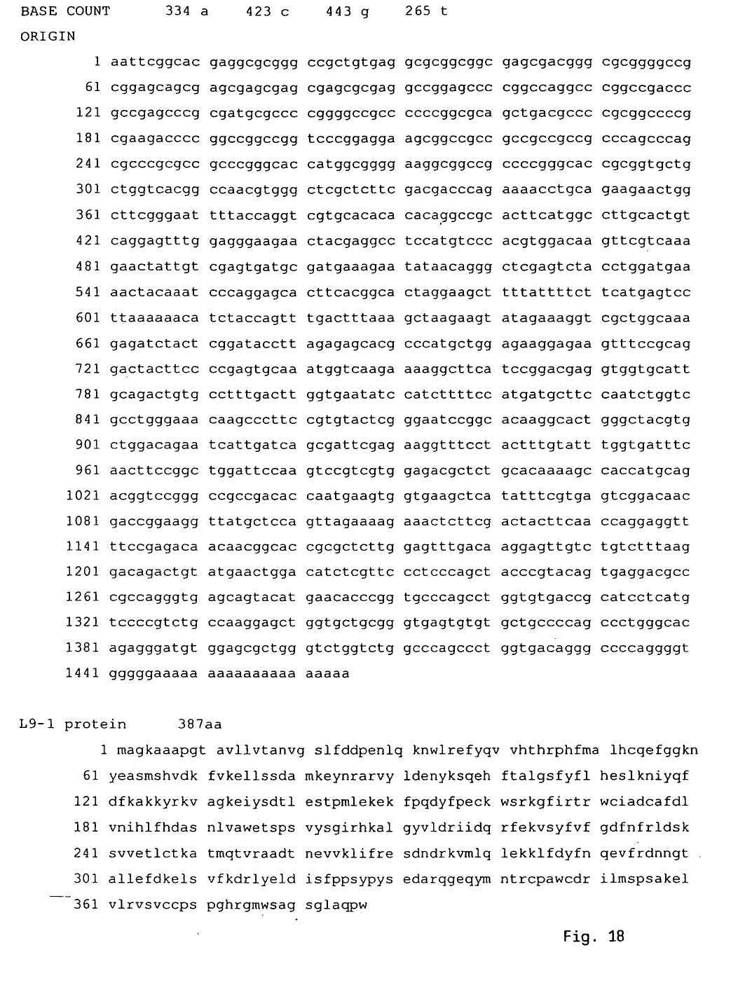

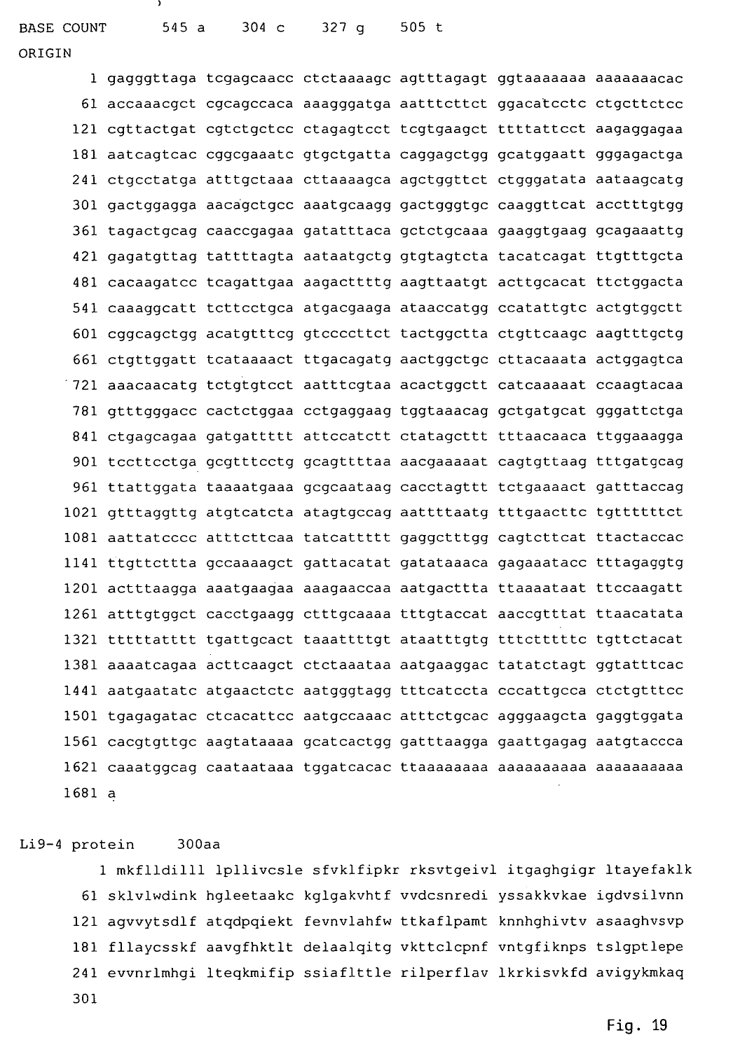

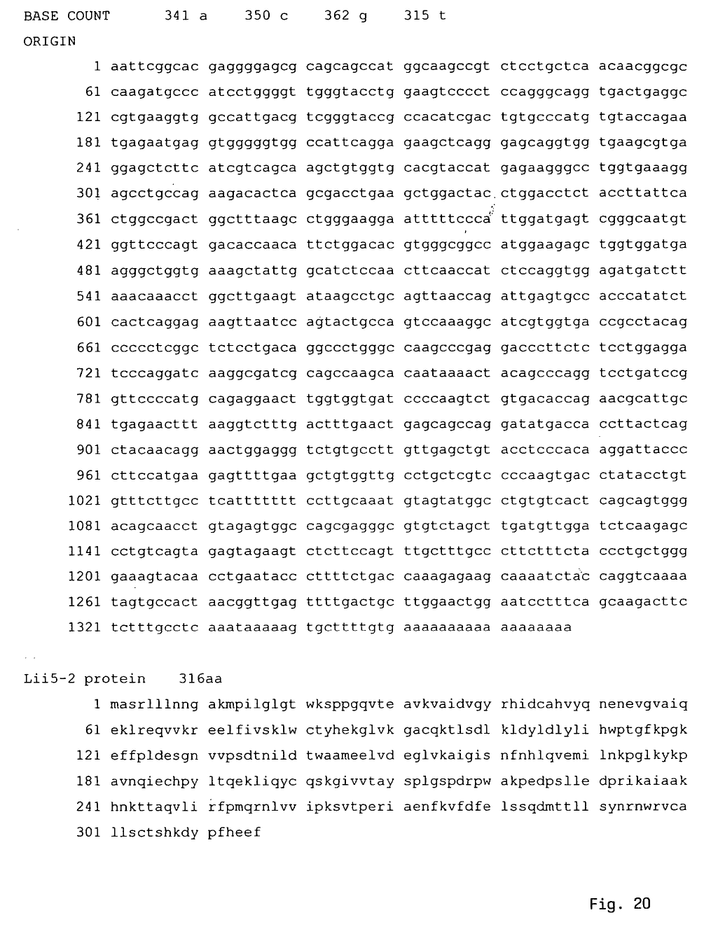

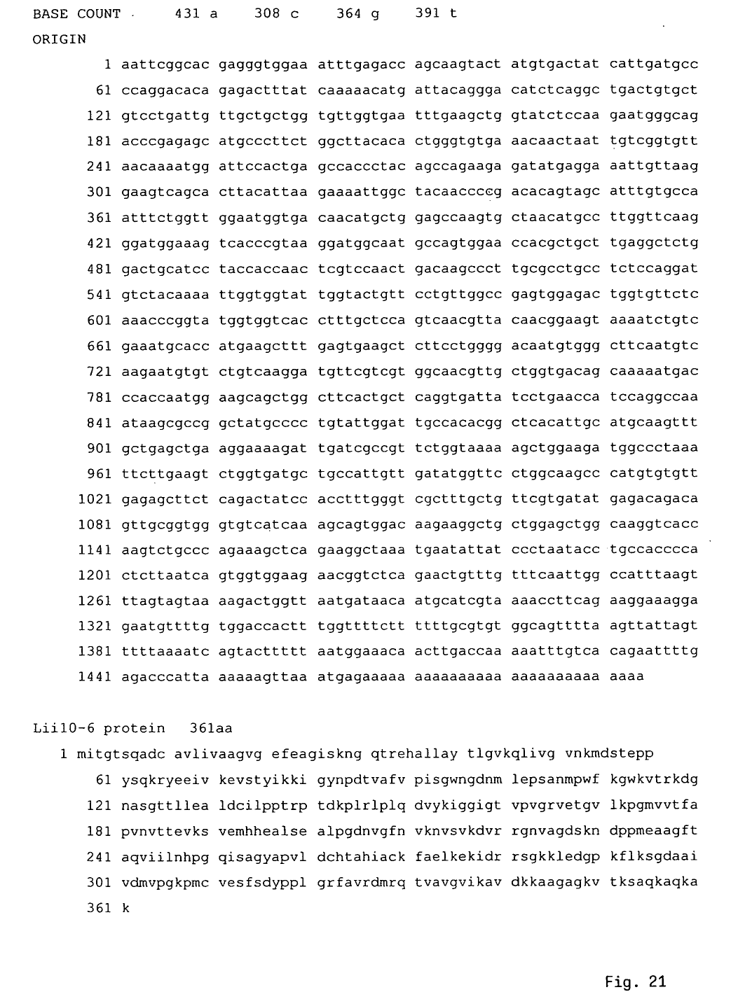

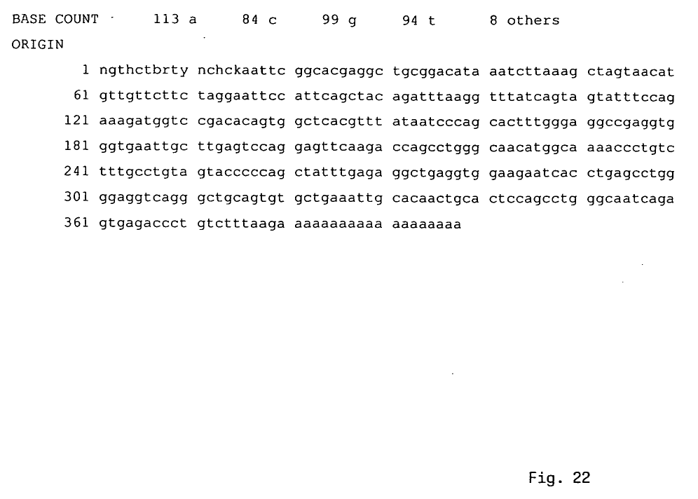

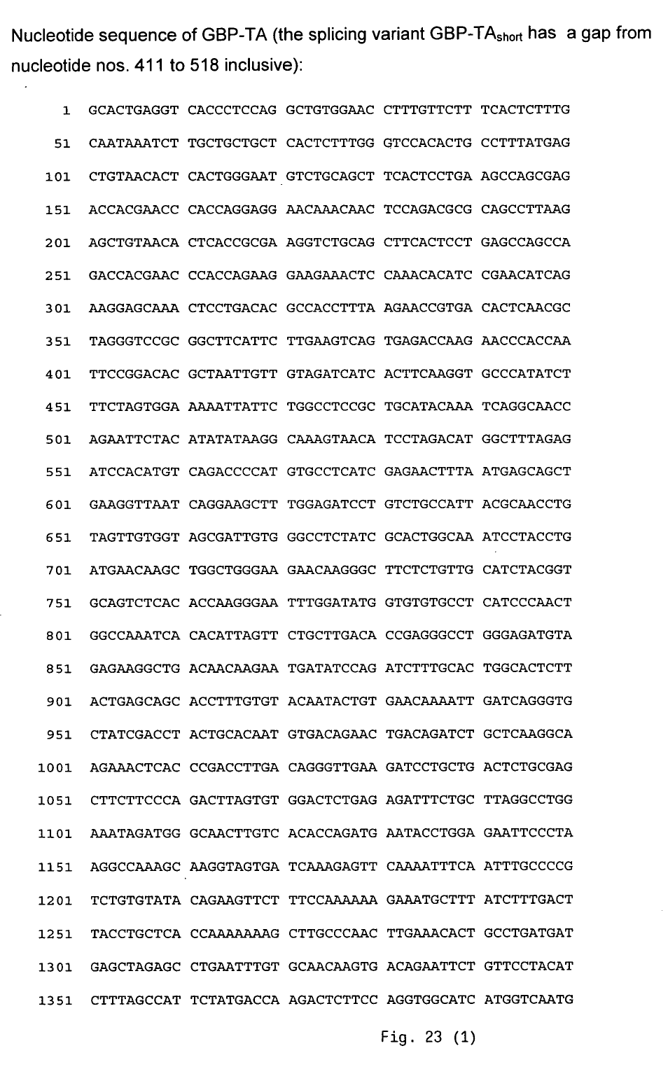

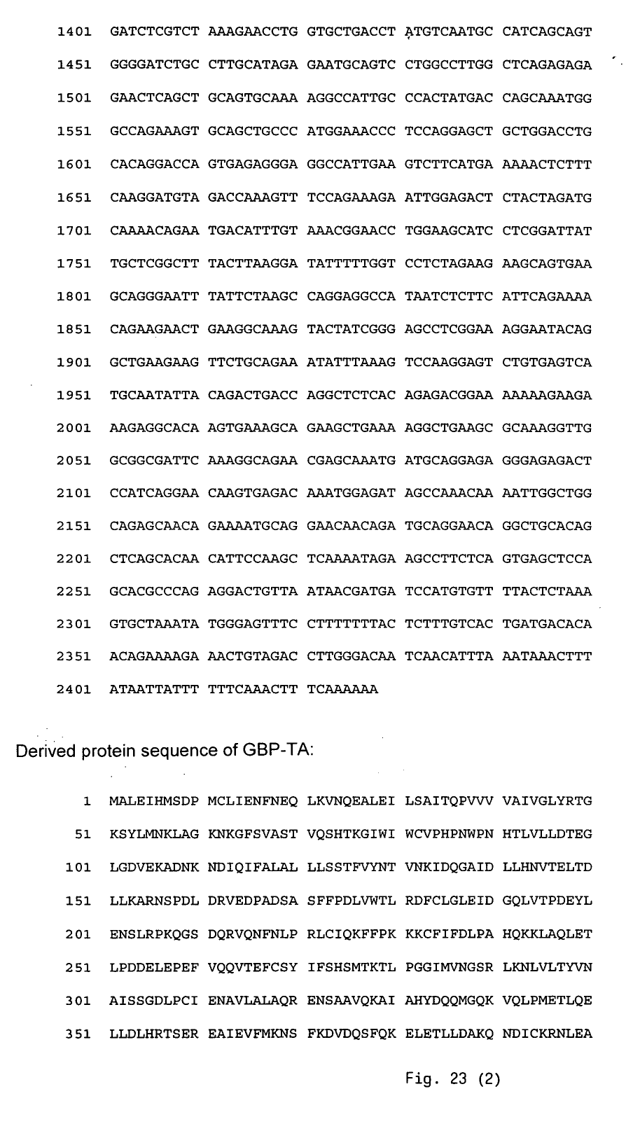

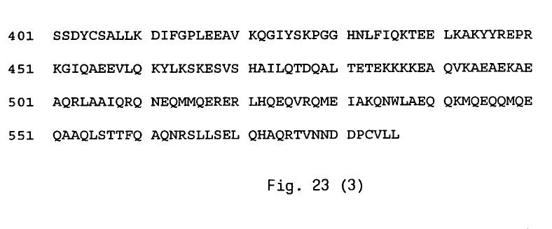

The subject matter of the present invention is thus a diagnostic composition containing at least one nucleic acid whose modified expression is associated with a tumoral disease, the nucleic acid sequence comprising se2-5 (FIG. 1), se20-10 (FIG. 2), se57-1 (FIG. 3), se70-2 (FIG. 4), Lg1-2 (FIG. 5), se1-1 (FIG. 6), se2-1 (FIG. 7), se2-2 (FIG. 8), se14-3 (FIG. 9), se20-4 (FIG. 10), se20-7 (FIG. 11), se20-9 (FIG. 12), se33-1 (FIG. 13), se37-2 (FIG. 14), se89-1 (FIG. 15), L14-2 (FIG. 16), L15-7 (FIG. 17), Li9-1 (FIG. 18), Li9-4 (FIG. 19), Lii5-2 (FIG. 20), Lii10-6 (FIG. 21), Liii4-5 (FIG. 22) or GBP-TA (FIG. 23).

The subject matter of the present invention also relates to a medicament containing a nucleic acid sequence whose modified expression is associated with a tumoral disease, the nucleic acid sequence comprising se20-10 (FIG. 2), se57-1 (FIG. 3), Lg1-2 (FIG. 5), se1-1 (FIG. 6), se2-1 (FIG. 7), se2-2 (FIG. 8), se14-3 (FIG. 9), se20-4 (FIG. 10), se20-7 (FIG. 11), se20-9 (FIG. 12), se33-1 (FIG. 13), se37-2 (FIG. 14), L14-2 (FIG. 16), L15-7 (FIG. 17), Li9-1 (FIG. 18), Li9-4 (FIG. 19), Lii5-2 (FIG. 20), Lii10-6 (FIG. 21), Liii4-5 (FIG. 22) or GBP-TA (FIG. 23).

The present invention also relates to an above defined diagnostic composition or a medicament, the nucleic acid sequence whose modified expression is connected with a malignant tumoral disease comprising a nucleic acid sequence (a) which on account of the degeneration of the genetic code differs from an above nucleic acid sequence shown in FIGS. 1 to 23 as regards the codon sequence;

(b) which hybridizes with a nucleic acid sequence according to any of FIGS. 1 to 23 or according to (a); or

(c) which is a fragment, an allelic variant or another variant of one of the above defined nucleic acid sequences.

The term “hybridizing nucleic acid sequence” refers to a nucleic acid sequence which under normal conditions, in particular 20° C. below the melting point of the nucleic acid, hybridizes with a nucleic acid sequence shown in the figures. The term “hybridize” used in the present invention refers to conventional hybridization conditions, preferably to hybridization conditions using as a solution 5×SSPE, 1% SDS, 1× Denhardt's solution and/or having a hybridization temperature between 50° C. and 70° C., preferably of 65° C. Following hybridization, a wash step is preferably carried out first with 2×SSC, 1% SDS and then with 0.2×SSC at temperatures between 50° C. and 70° C., preferably of 65° C. (for a definition of SSPE, SSC and Denhardt's solution see Sambrook et al., Molecular Cloning: A Laboratory Manual, 2^{nd} edition, Cold Spring Harbor Laboratory Press, Cold Spring Harbor N.Y. (1989)). Stringent hybridization conditions, as described in Sambrook et al., supra, for example, are preferred.

The terms “another variant” or “fragment” used in the present invention comprise nucleic acid sequences which differ from the sequences indicated in the figures or the above hybridizing sequences by deletion(s), insertion(s), substitution(s) and/or other modifications known in the art and a fragment of the original nucleic acid sequence, respectively, the protein encoded by these nucleic acid sequences also comprising one or more properties described above or in the examples. Allele variants are also counted thereamong. The variants show homology of at least 70%, at least 80%, preferably at least 90%, most preferably at least 95%, 96%, 97%, 98% or 99% with respect to the claimed sequences. Methods of producing the above modifications in the nucleic acid sequence are known to a person skilled in the art and are described in standard works of molecular biology, e.g. in Sambrook et al., supra. The person skilled in the art can also determine whether a protein encoded by a nucleic acid sequence modified in this way still has the desired biological properties. Above all in connection with diagnostic applications and/or compositions which use one of the above nucleic acid sequences, the term “fragment” relates to a fragment which has a length of at least 12, preferably at least 20 and more preferably at least 25, nucleotides.

In a preferred embodiment the above defined nucleic acid molecule is a cDNA. In another preferred embodiment, the nucleic acid sequence is a genomic DNA which is preferably derived from a mammal, e.g. a human being. Screening methods based on nucleic acid hybridization permit the isolation of the genomic DNA molecules according to the invention from any organism or derived genomic DNA libraries, probes being used which contain the nucleic acid sequence indicated in the figures or a fragment thereof.

The nucleic acid sequences can also be inserted in a vector or expression vector. A person skilled in the art is familiar with examples thereof. In the case of an expression vector for E. coli, these are e.g. pGEMEX, pUC derivatives (e.g. pUC8), pBR322, pBlueScript, pGEX-2T, pET3b and pQE-8. For the expression in yeast, e.g. pY100 and Ycpad1 have to be mentioned while e.g. pKCR, pEFBOS, cDM8 and pCEV4 have to be indicated for the expression in animal cells. The baculovirus expression vector pAcSGHisNT-A is especially suited for the expression in special insect cells. In a preferred embodiment, the nucleic acid is functionally linked in the vector with regulatory elements permitting the expression thereof in prokaryotic or eukaryotic host cells. Such vectors contain along with the regulatory elements, e.g. a promoter, typically a replication origin and specific genes permitting the phenotypic selection of a transformed host cell. The regulatory elements for the expression in prokaryotes, e.g. E. coli, comprise the lac-, trp promoter or T7 promoter and those for the expression in eukaryotes comprise the AOX1 or GAL1 promoter in yeast and the CMV, SV40, RVS-40 promoter, CMV or SV40 enhancer is used for the expression in animal cells. Further examples of suitable promoters are the metallothionein I and the polyhedrin promoters.

Suitable vectors are in particular also expression vectors based on T7 for the expression in bacteria (Rosenberg et al., Gene 56 (1987), 125) or pMSXND for the expression in mammalian cells (Lee and Nathans, J. Biol. Chem. 263 (1988), 3521).

General methods known in this special field can be used for constructing vectors or plasmids containing the above nucleic acid sequences and suitable control sequences. These methods comprise e.g. in vitro recombination methods, synthetic methods and in vivo recombination methods, as described in Sambrook et al., supra, for example.

Host organisms can be transformed with the above described vectors. These transformants comprise bacteria, yeasts, insect and further animal cells, preferably mammalian cells. The E. coli strains HB101, DH1, x1776, JM101, JM109, BL21, XL1Blue and SG 13009, the yeast strain Saccharomyces cerevisiae and the animal cells L, 3T3, FM3A, CHO, COS, Vero, HeLa as well as the insect cells sf9 are preferred. Methods of transforming these host cells for the phenotypic selection of transformants and for the expression of the above nucleic acid sequences using the above described vectors are known in this special field.

The above mentioned nucleic acids are particularly suited as antigen-coding structure for therapeutic purposes. The objective is to stimulate the immune system and eliminate tumor cells which are identified via a nucleic acid. In this connection there are various possibilities, e.g. giving the patient the naked DNA by injection. To this end, a plasmid having a very active promoter and at least one nucleic acid according to the invention, comprising in particular se20-10, se57-1, Lg1-2, se1-1, se2-1, se2-2, se14-3, se20-7, se20-9, se33-1, se37-2, L14-2, L15-7, Li9-1, Li9-4, Lii5-2, Lii10-6, Liii4-5 or GBP-TA, is introduced e.g. into the muscle or intradermally by injection.

Furthermore, the nucleic acid sequence can be inserted in the vector for the purpose of recombinant production and also, using vectors, to introduce the DNA by injection into patients where this DNA encodes an antigen for therapeutic purposes. The objective is that the cells take up the plasmid, produce antigens, present individual peptides via HLA molecule, thus causing a cytotoxic T-cell immune response which shall then result in combating tumor cells. This procedure is generally described in Conry et al., Clinical Cancer Research 4, pp. 2903-2912 (1998). The gene gun method is an alternative which is described in Fynan et al., Proc. Natl. Acad. Sci. U.S.A. 90, pp. 11478-11482 (1993). Here, the nucleic acid is introduced into in vivo antigen-presenting cells (APCs), e.g. dendritic cells, by means of a vector for the HLA-presentation of the encoded protein. The vector containing the nucleic acid according to the invention can in this case be injected differently:

a) Lipid- or liposome-packed DNA or RNA, e.g. generally described by Nabel et al., Proc. Natl. Acad. Sci. U.S.A. 93, pp. 15388-15393 (1996).

b) Using a bacterium as a transport vehicle for the expression vector. Suitable bacteria are e.g. (attenuated) listerias [e.g. Listeria monocytogenes], salmonella strains [e.g. Salmonella spp.]. This technique is generally described by Medina et al., Eur.

J. Immunol. 29, pp. 693-699 (1999) as well as Guzman et al., Eur. J. Immunol. 28, pp. 1807-1814 (1998).

Reference is also made to WO 96/14087; Weiskirch et al., Immunological Reviews 158, pp. 159-169 (1997) and US-A-5,830,702.

c) By means of gene gun (Williams et al., Proc. Natl.

Acad. Sci. U.S.A. 88, pp. 2726, 2730, 1991).

In a preferred embodiment, the vector containing the above nucleic acid sequences is a virus, e.g. an adenovirus, vaccinia virus or an AAV virus, which is of use for a gene therapy. Retroviruses are particularly preferred. Examples of suitable retroviruses are MoMuLV, HaMuSV, MuMTV, RSV or GaLV. Furthermore, the above mentioned viruses and the fowlpox virus, canarypox virus, influenza virus or sindbis virus can also be used as a basis for a vaccine. Such new vaccines which when administered to the patient give anti-tumor immunity, are described e.g. in N. Restifo, Current Opinion in Immunology 8, pp. 658-663 (1996) or Ying et al., Nature Medicine 5(7), p. 823 et seq., (1999). For the purpose of genetic engineering, the above nucleic acid sequences can also be transported to the target cells in the form of colloidal dispersions. They comprise e.g. liposomes or lipoplexes (Mannino et al., Biotechniques 6 (1988), 682).

In order to produce tumor immunity it is also preferred to transfect an above nucleic acid sequence in antigen-presenting cells and introduce it into the patient by injection. Here, a plasmid is introduced in vitro into an antigen-presenting cell (APCs), e.g. dendritic cells, which then produce antigens and present individual peptides via HLA molecules. In this connection, the plasmid DNA can be introduced into the antigen-presenting cells in various ways:

(a) as a naked DNA, e.g. by means of gene gun or electroporation;

(b) as a lipid- or liposome-packed DNA or RNA (Nair et al., Nature Biotechnology 16, p. 364 et seq. (1998));

(c) with a virus as a vector (Kim et al., J. of Immunotherapy 20(4), pp. 276-286 (1997));

(d) with a bacterium as a transport vehicle for the expression vector (Medina et al., Eur. J. Immunol. 29, pp. 693-699 (1999); Guzman et al., Eur. J. Immunol 28, pp. 1807-1814 (1998)).

It is also possible to prepare the protein encoded by an above nucleic acid and correlated with the presence of a malignant tumoral disease. The method preferably used for this purpose comprises culturing the above described host cells under conditions which permit the expression of the protein (preferably stable expression) and collecting the protein from the culture. Suitable methods for the recombinant production of the protein are generally known (see e.g. Holmgren, Annu. Rev. Biochem. 54 (1985), 237; LaVallie et al., Bio/Technology 11 (1993), 187; Wong, Curr. Opin. Biotech. 6 (1995), 517; Romanos, Curr. Opin. Biotech. 6 (1995), 527; Williams et al., Curr. Opin. Biotech. (1995), 538; and Davies, Curr. Opin. Biotech. 6 (1995), 543). Suitable purification methods (e.g. preparative chromatography, affinity chromatography, e.g. immunoaffinity chromatography, HPLC, etc.) are also generally known. In this connection, it should be noted that the above mentioned protein can be modified according to conventional methods known in this special field. These modifications comprise substitutions, insertions or deletions of amino acids which modify the structure of the protein while substantially maintaining its biological activity. The substitutions preferably comprise “conservative” substitutions of amino acid residues, i.e. substitutions by biologically similar residues, e.g. the substitution of a hydrophobic residue (e.g. isoleucine, valine, leucine, methionine) by another hydrophobic residue, or the substitution of a polar residue by another polar residue (e.g. arginine by lysine, glutamic acid by asparagic acid, etc.). Deletions may results in the preparation of molecules which are markedly reduced in size, i.e. which lack e.g. amino acids at the N-terminus or C-terminus.

Injections of at least one of the proteins or one or several of the peptides derived therefrom are also suited for the desired anti-tumor vaccination. For this purpose, HLA-dependent peptide fragments are determined from the sequence of the protein according to the invention by means of either corresponding computer programs or experiments (e.g. phagocytotic picture of the whole protein, thereafter analysis of the presenting molecules). They are produced artificially by means of methods known to the person skilled in the art and then given the patient by injection (where necessary, with immune system-stimulating factors, e.g. interferons, interleukins, etc.). The objective behind this treatment is that the APCs take up the peptides, present them, thus stimulating in vivo the production of tumor-specific cytotoxic T-cells. This principle was generally described by Melief et al., Current Opinion in Immunology 8, pp. 651-657 (1996).

Just as described above, in place of the vector the above protein or fragment thereof can also be loaded in vitro onto APCs. The loaded cells are then introduced into the patient, e.g. into the lymph nodes, by injection and directly provide for the stimulation and proliferation of tumor-specific cytotoxic T-cells (Nestle et al., Nature Medicine 4(3), p. 328 et seq. (1998); Schadendorf et al., in: Burg, Dummer, Strategies for Immunointerventions in Dermatology, Springer Verlag, Berlin Heidelberg, pp. 399-409, 1997). Regarding the vaccination it may be advantageous to modify individual amino acids with respect to the wild-type antigen, as described above, since under certain circumstances this might increase the bond and improve the effectiveness (Clay et al., The Journal of Immunology 162, pp. 1749-1755, 1999).

As regards the above therapeutic measures particularly preferred is at least one nucleic acid sequence which comprises the nucleic acid sequence se20-10 (FIG. 2), se57-1 (FIG. 3), Lg1-2 (FIG. 5), se1-1 (FIG. 6), se2-1 (FIG. 7), se2-2 (FIG. 8), se 14-3 (FIG. 9), se20-7 (FIG. 11), se20-9 (FIG. 12), se33-1 (FIG. 13), se37-2 (FIG. 14), L14-2 (FIG. 16), L15-7 (FIG. 17), Li9-1 (FIG. 18), Li9-4 (FIG. 19), Lii5-2 (FIG. 20), Lii10-6 (FIG. 21), Liii4-5 (FIG. 22) or GBP-TA or a protein encoded thereby or a fragment thereof.

The present invention also relates to antibodies which detect specifically the above described proteins (tumor antigens). The antibodies may be monoclonal, polyclonal or synthetic antibodies or fragments thereof, e.g. Fab, Fv or svFv fragments. They are preferably monoclonal antibodies. For the preparation thereof it is favorable to immunize animals—particularly rabbits or chickens for a polyclonal antibody and mice for a monoclonal antibody—with an above (fusion) protein or with fragment(s) thereof. Further “boosters” of the animals can be effected with the same (fusion) protein or with fragments thereof. The polyclonal antibody may then be obtained from the animal serum or egg yolk. The antibodies according to the invention can be prepared according to standard methods, the protein encoded by the above mentioned nucleic acid sequences or a synthetic fragment thereof serving as an immunogen. Monoclonal antibodies may be prepared by the method described by Kohler and Milstein (Nature 256 (1975), 495) and Galfré (Meth. Enzymol. 73 (1981), 3), for example, murine myeloma cells being fused with spleen cells derived from immunized mammals. These antibodies can be used for the immunoprecipitation of the above discussed proteins or for the isolation of related proteins from cDNA expression libraries, for example. The antibodies can be bound in immunoassays in the liquid phase or to a solid carrier, for example. Here, the antibodies may be labeled in different ways. Suitable markers and labeling methods are known in the special field. Examples of immunoassays are ELISA and RIA.

Furthermore, along with their diagnostic suitability the antibodies can also be used therapeutically. Here, e.g. a protein encoded by the above nucleic acid sequences serves as a target for bispecific antibodies. Reference is made in this connection to Kastenbauer et al., Laryngorhinootologie 78(1), pp. 31-35 (1999) and Cao et al., Bioconj. Chem. 9(6), pp. 635-644 (1998). The antibodies according to the invention are suited to catch antigen which is overexpressed in tumors, for example, so as to inhibit the tumor growth, since there is reference that in some cases the occurrence of tumor antigens does not only indicate the presence of malignant tumors but actively promotes the tumor growth.

It is also possible to inhibit the translation of the above nucleic acid sequences which have increased expression in tumors using antisense DNA (RNA) or ribozymes so as to exert specifically a therapeutic effect on these nucleic acid sequences or genes. In the corresponding tumor cells, RNA/DNA hybrids form which prevent the transcription in this way and—in the case of the antisense RNA—simultaneously effect a degradation of the hybrids (and thus the RNA) by RNase H (Scanlon et al., The Faseb Journal 9, pp. 1288-1296, 1995).

The present invention thus relates to a medicament or a diagnostic composition which contains the above described nucleic acid sequences, vectors, proteins, antibodies, etc., or combinations thereof and to the use thereof for diagnosis and/or therapy. They are used preferably for the diagnosis or treatment of malignant tumoral diseases, in particular CTCL. The provision of a vaccination agent which, as described above, is based on either the nucleic acid sequence or the protein/peptide is preferred. Herein, the diagnostic composition is suited to detect a malignant tumoral disease and also to carry out a follow-up, e.g. to accompany a therapy.

The above medicaments additionally contain, where appropriate, a pharmaceutically compatible carrier. Suitable carriers and the formulation of such medicaments are known to the person skilled in the art. Suitable carriers are e.g. phosphate-buffered common salt solutions, water, emulsions, e.g. oil/water emulsions, wetting agents, sterile solutions, etc. The medicaments can be administered orally or parenterally. The methods for the parenteral administration comprise the topical, intra-arterial, intramuscular, subcutaneous, intramedullary, intrathekal, intraventricular, intravenous, intraperitoneal or intranasal administration. The suitable dosage is determined by the attending physician and depends on various factors, e.g. the patient's age, sex and weight, the disease stage, the kind of administration, etc.

A above nucleic acid sequence can also be used as a probe to isolate DNA molecules which originate from another species or another organism, for example, and code for a protein having the same biological activity. For this purpose, the probe preferably has a length of at least 20, in particular preferably at least 25 bases. Suitable detection methods based on the hybridization are known to the person skilled in the art, e.g. Southern or Northern blot. The person skilled in the art is also familiar with suitable labelings for the probe, which comprise e.g. labeling with radioisotopes, bioluminescence, chemiluminescence, and fluorescence markers, metal chelates, enzymes, etc.

In addition, this can also be made by PCR (Wiedmann et al., PCR Methods Appl. 3, pp. 551-564 (1994); Saiki et al., Nature 324, pp. 163-166 (1986)) or ligase chain reaction (LCR) (Taylor et al., Curr. Opin. Biotechnol. 6, pp. 24-29 (1995); Rouwendal et al., Methods Mol. Biol., pp. 149-156 (1996)), the primers being derived from the sequence in the figures and suitable primers (as regards length, complementarity with respect to the matrix, the region to be amplified, etc.) being designable by the person skilled in the art according to common methods.

Besides, the present invention relates to a method for the diagnosis of malignant tumoral diseases, in vitro, the above nucleic acid sequences or fragments thereof being used as a probe.

With respect to the diagnostic method it is preferred to provide the nucleic acids and/or proteins in the form of an ELISA kit, a protein chip, nucleic acid chip or membrane loaded with DNA, RNA or protein.

In connection with the above-mentioned method, it is possible to use methods known to the person skilled in the art as regards the preparation of DNA or RNA from biological samples, the restriction digestion of the DNA, the separation of the restriction fragments on gels separating according to size, e.g. agarose gels, the preparation and labeling of the probe and the detection of hybridization, e.g. by means of Southern blot or in situ hybridization.

This diagnostic method is preferably a method comprising the steps of:

isolating nucleic acid from the patient,

carrying out LCR or PCR with suitable primers or a hybridization analysis with one or more suitable probes based on a nucleic acid sequence of the figures;

detecting an amplified product or a hybridization as an indication of the presence (or absence) of a tumoral disease (as a function of whether the respective nucleic acid sequence is expressed to a greater or lesser extent as compared to the control tissue (or is not expressed) in the tumor).

Here, primers are used which flank an above discussed nucleic acid sequence or suitable partial regions. For this purpose, amplification products of mRNA from the respective tissue are of diagnostic significance, which products differ as regards the occurrence of tumor-specific, in particular CTCL-specific, bands from the amplification products of mRNA from healthy tissue.

In an alternative preferred embodiment, a method can be used which comprises the steps of:

isolating RNA from the patient,

carrying out a Northern blot analysis with one or more suitable probes,

comparing the concentration and/or length of the corresponding mRNA of the patient's sample with an mRNA from a healthy person, an increased or lowered concentration of mRNA (as a function of the corresponding marker; see Table 4) as compared to the control mRNA from normal tissue being an indication of a tumoral disease, in particular CTCL.

In this method, it is possible to use methods known to the person skilled in the art as regards the preparation of whole RNA or poly(A)+RNA from biological samples, the separation of the RNAs on gels separating according to size, e.g. denaturing agarose gels, the preparation and labeling of the probe and the detection via Northern blot.

In another alternative embodiment, a possible tumoral disease can also be diagnosed by a method comprising the steps of:

obtaining a cell sample from the patient,

contacting the resulting cell sample with one or more proteins encoded by the nucleic acid sequences according to the invention or fragments thereof as probe(s) under conditions permitting the binding of antibodies, the presence of antibodies in the cell sample being an indication of a tumoral disease, in particular CTCL.

This detection can also be carried out using standard methods with which the person skilled in the art is familiar. He also knows cell digestion methods which permit the isolation of the antibodies in a way such that they can be contacted with the antigen. The bound antibody is preferably detected by means of immunoassays, e.g. Western blot, ELISA, FACS or RIA or immunohistochemical methods. It is preferably carried out by means of ELISA or dot blot. For establishing an ELISA, the above nucleic acid sequences or fragments thereof can be cloned into expression plasmids and the corresponding proteins can be prepared recombinantly, preferably as fusion proteins with a His-Tag, which facilitates the purification thereof. The proteins are then applied to membranes or other suitable surfaces, optionally fixed and incubated with adequately diluted patient serums. After the common wash steps, an incubation is carried out with a secondary labeled antibody according to routine methods for detecting the bound patient's antibodies. The patient serums are preferably incubated with a plurality of marker proteins (antigens) since the detection of the presence (or absence) of different antibodies better indicates the underlying tumoral disease and possibly enables a classification according to the disease stage.

The present invention also relates to a kit for carrying out the diagnostic method according to the invention, which contains the antibody according to the invention or a fragment thereof, an above protein (or a peptide derived therefrom), a nucleic acid sequence according to the invention (as a probe) or a primer suited for PCR or LCR, for example, and based on the nucleic acid sequences according to the invention (or a primer pair), optionally in combination with a suitable detection means.

Depending on the development of the diagnostic method to be carried out with the kit according to the invention, the compounds contained in the kit (nucleic acid molecules, proteins, antibodies or fragments thereof) can be immobilized on a suitable carrier, i.e. in the form of a chip or be bound on a membrane.

All of the above mentioned proteins are detected serologically only by antibodies from the serums of tumor patients but not from the serums of control persons and are thus serologically tumor-specific. Since this specificity is not limited to a tumor type these proteins and antibodies are very well suited to make a distinction between malignity and non-malignity at all. An advantage in this connection is to carry out a study with more than one of the above mentioned tumor markers, i.e. a combination of tumor markers, and chose a therapeutic approach dependent thereon. This means that the medicament according to the invention should also contain more than one of the above mentioned nucleic acids, proteins or antibodies.

The invention is now described in more detail by means of the figures in which:

FIG. 1 shows the nucleic acid sequence of se2-5 and an ORF derived therefrom,

FIG. 2 shows the nucleic acid sequence of se20-10 and an ORF derived therefrom,

FIG. 3 shows the nucleic acid sequence of se57-1 and an ORF derived therefrom,

FIG. 4 shows the nucleic acid sequence of se70-2 and an ORF derived therefrom,

FIG. 5 shows the nucleic acid sequence of Lg1-2 and an ORF derived therefrom,

FIG. 6 shows the nucleic acid sequence of se1-1 and an ORF derived therefrom,

FIG. 7 shows the nucleic acid sequence of se2-1 and an ORF derived therefrom,

FIG. 8 shows the nucleic acid sequence of se2-2 and an ORF derived therefrom,

FIG. 9 shows the nucleic acid sequence of se14-3 and an ORF derived therefrom,

FIG. 10 shows the nucleic acid sequence of se20-4 and an ORF derived therefrom,

FIG. 11 shows the nucleic acid sequence of se20-7 and an ORF derived therefrom,

FIG. 12 shows the nucleic acid sequence of se20-9 and an ORF derived therefrom,

FIG. 13 shows the nucleic acid sequence of se33-1 and an ORF derived therefrom,

FIG. 14 shows the nucleic acid sequence of se37-2 and an ORF derived therefrom,

FIG. 15 shows the nucleic acid sequence of se89-1 and an ORF derived therefrom,

FIG. 16 shows the nucleic acid sequence of L14-2 and an ORF derived therefrom,

FIG. 17 shows the nucleic acid sequence of L15-7 and an ORF derived therefrom,

FIG. 18 shows the nucleic acid sequence of Li9-1 and an ORF derived therefrom,

FIG. 19 shows the nucleic acid sequence of Li9-4 and an ORF derived therefrom,

FIG. 20 shows the nucleic acid sequence of Lii2-5 and an ORF derived therefrom,

FIG. 21 shows the nucleic acid sequence of Lii10-6 and an ORF derived therefrom,

FIG. 22 shows the nucleic acid sequence of Liii4-5 and an ORF derived therefrom,

FIG. 23 shows the nucleic acid sequence of GBP-TA and an ORF derived therefrom,

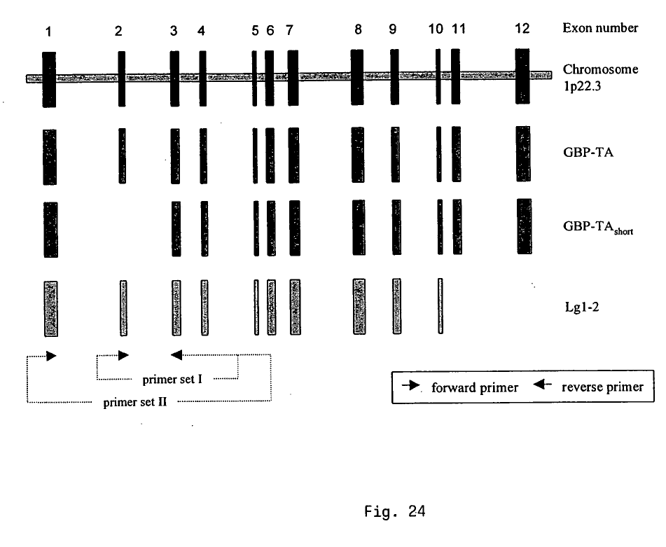

FIG. 24 shows the localization of GBP-TA, GBP-Taₛₕₒᵣₜ and Lg1-2 on chromosome 1p22.3. The primers used for distinguishing the splicing variants are drawn in.

Primer set I: tgt tgt aga tca ctt caa ggt gc (forw.)

cca tat cca aat tcc ctt ggt gtg ag (re.) annealing temperature 63° C.

Primer set II: aga agg aag aaa ctc caa aca cat cc (forw.)

cca tat cca aat tcc ctt ggt gtg ag (re.) annealing temperature 48° C.

The invention is now described below with reference to the examples.

As to the methods used reference is made along with the methods described in Example 1 to Sambrook, J. Fritsch, E. F. and Maniatis, T. (Molecular cloning; a laboratory manual; second edition; Cold Spring Habor Laboratory Press, 1989) and Current Protocols in Molecular Biology (John Wiley and Sons, 1994-1998), the below methods, in particular the screening of cDNA libraries, preparation of DNA or RNA, PCR, RT-PCR or Northern blot, being sufficiently known to, and mastered by, the person skilled in the art.

### EXAMPLE 1

### General Method

### (A) Tissues and Serums

Serums and tumor tissues were obtained in diagnostic or therapeutic routine methods with the patient's consent (and the permission of the competent ethics committee). The tissues and sera were stored at −20° C. or −80° C.

### (B) Preparation of cDNA Libraries

mRNA was extracted from testis samples using a kit for RNA isolation (RNeasy midi kit; Qiagen, Hilden, Germany) and then a kit for mRNA isolation (oligotex mRNA kit; Qiagen) in accordance with the manufacturer's recommendations. A total of 10.4 μg mRNA were used for the construction of the λ-ZAP expression library (UNI-ZAP# XR custom cDNA library; Stratagene, La Jolla, Calif., U.S.A.). The cDNA library consisted of 10⁶ primary recombinants having an insertion length of over 0.4 kbp and was amplified to give 10¹⁰ plaque-forming units (pfu). 4.8 μg mRNA from different samples of cutaneous T-cell and B-cell lymphomas were used for the construction of the CTCL library. The number of primary recombinants was 6×10⁷. The procedure was analogous to the previously described procedure used in the case of the testis library.

### (C) Immunoscreening

Immunoscreening was carried out as described in Sahin et al. (PNAS U.S.A. 92 (1995), 11810-11813) and Tureci et al. (Cancer Res. 56 (1996), 4766-4772). All of the serums were diluted in Tris-buffered saline (TBS with 0.2% milk powder, pH 7.5) and pre-absorbed with E. coli proteins (broken up mechanically or lyzed by phages without insertion). E. coli transduced using recombinant λ-ZAP phages were plated onto NZY agar at a concentration of 2000 plaques/plate, and the expression of the recombinant proteins was induced by means of isopropyl-β-D-thiogalactoside. The plates were incubated at 37° C. overnight and the proteins were transferred at 37° C. for 4 hours on nitrocellulose membranes and bound. The membranes were washed with TBS which contained Tween-20™ (0.05%), saturated with 5% milk powder in TBS and incubated with serums (either from patients or as a control from healthy persons) at a final concentration of 1/100. Reactive proteins were identified with an alkaline phosphatase-coupled secondary antibody (goat anti-human IgC, Fc fragment; Dianova, Hamburg, Germany) and made visible by means of 5-bromo-4-chloro-3-indolylphosphate and nitroblue tetrazolium. Positive phagemides were further investigated by means of serums from patients suffering from Mycosis fungoides (n=15) and Sézary syndrome (n=3) and healthy persons as a control (n=10). Positive phagemides were subcloned for monoclonality and subjected to an in vivo excision of the pBluescript plasmid (in accordance with the protocol from the manufacturer of Genbank, Stratagene, La Jolla, Calif., U.S.A.). DNA was isolated in accordance with the manufacturer's protocol (QIAprep spin miniprep; Qiagen). The size of the insertions was analyzed by SmaI/KpnI cleavage and gel electrophoresis. Sequencing was carried out by means of an automatic fluorescence sequencing device (model 377; Perkin-Elmer/Applied Biosystems, Forster System, Calif., U.S.A.) and the Dye-Terminator method in accordance with the manufacturer's instructions (ABI PRISM Big Dye Ready Reaction Terminator Cycle Sequencing Kit; Perkin-Elmer). Primers were synthesized chemically. The sequences of the clones were completely determined on both complementary strands.

### (D) Tumor Tissues and Cell Lines

Tissue samples obtained from 17 CTCL patients served as a source for the preparation of tumor cDNA: 13 Mycosis fungoides (stages Ib to IVb), mainly IIb), 2 Sézary syndromes (stage III), 1 T-zone lymphoma (stage IVb) and 1 CD30+ CTCL (stage IIb). Furthermore, cDNAs of the following 4 CTCL cell lines were prepared: My-La (Mycosis fungoides; Kaltoft et al., In Vitro Cell Dev. Biol. 28a (1992), 161-167), SeAx (Sézary syndrome; Kaltoft et al., Arch. Dermatol. Res. 280 (1988), 264-267), HH (lymphomatoid papulosis; ATCC No.: CRL-2105) and HuT-78 (Sézary syndrome; ATCC No.: TIB-161). Besides, cDNA was prepared from six leukemia cell lines (ARA-10, Jurkat, KG1, K562, Nalm-2 and SKW6.4 and 22 melanoma cell lines.

For analyzing the tissue distribution within normal tissues, control cDNAs were used in detail, which also comprised three fields of commercially available cDNAs (all from Clontech, Calif., U.S.A.): human multiple tissue cDNA field I, field II and human fetal MTC field. In addition, different commercially available whole RNAs for the preparation of further control cDNAs were produced by means of the above described method. Finally, cDNAS of three activated CD8+ T-cell lines (Möller et al., British Journal of Cancer 77 (1998), 1907-1916) also served as control T-cells.

### (E) RT-PCR

On account of the limited amount of RNA, RT-PCR was preferably used for studying the identified sequences within different normal tissues and tumor tissues. In selected cases, these studies were completed by means of Northern blot analyses. RT-PCR was carried out by means of “MJ Research PCT-200” (Biozym, Oldendorf, Germany) with a one-minute attachment at variable temperature and 35 cycles. All of the RT-PCRs were carried out in at least two independent experiments. As for the rest, the RNA isolation, RT-PCR and Northern blot analyses were carried out according to conventional standard methods and under standard conditions. The primer sequences and annealing temperatures for the different clones are indicated in below table 1:

| | | | Anneal. |
|---|---|---|---|
| Clone | Forward Primer | Reverse Primer | Temp. |
| se1-1 | gca aaa gca att aga cgc tac c | cac agc cct gtt ctt ctt tag c | 57° C. |
| se2-1 | gta cag cag aaa gca agc aac tga atg | gga aat tgg att cta aag cag ttc ctt c | 55° C. |
| se2-2 | cta tga atc caa gac caa agg c | ctc cac ttt ggt cct tgt tag c | 59° C. |
| se2-5 | acc cac gca gat ttg gaa tc | agg ctg atc act ggc tgt g | 59° C. |
| se14-2 | cct tat tgt aca ctg ggg ctt c | cag aca caa gga act gaa gta acg | 60° C. |
| se14-3 | cac tgc caa gat aga caa gca g | gct ctt atc cag gaa gtc cat g | 59° C. |
| se20-4 | tac agg atc tca gac ata tct cca tg | aaa tgt ctt ccc act gca taa tag tc | 59° C. |
| se20-7 | taa gga aac aat tca gtc aca taa gg | ctg tag ctt agc aat ttg ttct tct g | 59° C. |
| se20-9 | tta tga ggc tta gaa ttt caa cca c | aaa ggc ttt caa aac att ttt caa c | 59° C. |
| se20-10 | gta gag atc aga gag ttg tga cat ctg | tat tac ttt tca ctg tta cac tgc tgg | 59° C. |
| se33-1 | gcc aca gag aat gaa cca ctt aac | gag gga cta tca gtt gct gtt tg | 60° C. |
| se37-2 | gca tct aat aga acg cta cta cca cc | ctg tga gct atc acc tat cct tga g | 60° C. |
| se57-1 | gtg aca gtg acc aca gaa att ccc cc | cac gtt tct cag agc tgc tgc tcc | 63° C. |
| se70-2 | gct gca cag aaa acc tta ctt gtt tcc acc | ctc gta aat gca gaa atc tcc aat gcc c | 56° C. |
| se89-1 | tcc aca gcc tat tgg ctc act tgg ac | gcc ctt tag tgt gtc tgt aat tgg aat cag | 57° C. |
| RAP140 | tcc aca gcc tat tgg ctc act tgg ac | gca cac act gct cct cca cct gac | 57° C. |
| L14-2 | gct gct gct gtt tac aga aag gct cac | gga aag tta tcc aca gct act gag gac cc | 64° C. |
| L15-7 | tcc cct cca ttt aat ctc caa att cac cc | ctc agc att tgc cgc cgt aac tt | 62° C. |
| Li9-1 | gaa aac tac aaa tcc cag gag cac | ctc acg aaa tat gag ctt cac cac | 63° C. |
| Li9-4 | tta ctg atc gtc tgc tcc cta gag tcc | atc ttc tgc tca gtc aga atc cca tgc | 67° C. |
| Lg1 − 2 = | tgt tgt aga tca ctt caa ggt gc | cca tat cca aat tcc ctt ggt gtg ag | 63° C. |
| Primer set I | | | |
| (GBP-TA) | | | |
| GBP-TA | tgt tgt aga tca ctt caa ggt gc | cca tat cca aat tcc ctt ggt gtg ag | 48° C. |
| Primer set II | | | |
| Lii5-2 | tga gaa tga ggt ggg ggt gg | tgg gga acc gga tca gga c | 58° C. |
| Lii10-6 | gca tcc tac cac caa ctc gtc c | agt tct gag acc gtt ctt cca cc | 57° C. |
| Liii4-5 | gct gcg gac ata aat ctt aaa gc | agg gtc tca ctc tga ttg cc | 56° C. |

### (F) Northern Blot

10 μg whole RNA are separated electrophoretically on an MOPS gel and transferred to positively charged nylon membranes. The probe is labeled by means of the Roche High Primer Kit using α-³²P-dCTP. The non-incorporated nucleotides are removed by means of the Qiagen Removal Kit (Qiagen company, Hilden). The prehybridization is carried out at 60° C. for 1-2 hours. The hybridization is preferably carried out at 60° C. overnight. The prehybridization and hybridization solutions have the following composition: 10% dextran sulfate, 1% SDS, 10× Denhardt's reagent, 3×SSC. The subsequent wash step is carried out at 42° C. using 2×SSC/0.1% for 2×30 minutes and then at 65° C. using 0.2×SSC/0.1% SDS for 2×30 minutes. A Kodak X-Omat film is applied for an exposure time of 3-10 days.

### EXAMPLE 2

### Screening According to Positive Clones

About 1.9×10⁶ recombinant clones of a cDNA library obtained from normal testis tissue were screened with the serums from patients suffering from cutaneous T-cell lymphomas (CTCL) including Mycosis fungoides (MF) and Sézary syndrome. 28 clones representing 22 different ORFs and/or genes could be detected and they were further investigated as regards serological reactivity and molecular distribution. A secondary confirmation was carried out by the use of additional serums from patients having a positively diagnosed Mycosis fungoides (MF) (n=15) or Sézary syndrome (n=3) and of 10 control serums from healthy volunteers. The reactivity of the serums from the patients was associated with the tumor stage (maximum stage III) (Table 2). However, this was not statistically significant (X² test and Mann-Whitney U test), presumably because of the small number of serums having advanced tumor stages. The reactivity of the patient serums ranged from 11% to 71% of serums identifying recombinant clones.

TABLE 2
Number of positive clones in correlation with the
tumor stage of the serum donor

| | Primary screening | | | Secondary screening | | |
|---|---|---|---|---|---|---|
| | | Screened | | | | |
| Patient's | Number | plaques | | Number | Plaques | |
| tumor | of serums | (positive/ | | of serums | (positive/ | |
| stage | used | tota)⁽¹⁾ | Frequency⁽²⁾ | used | total) | Frequency⁽²⁾ |
| I | 6 | 1/524,000 | 0.2 × 10⁻⁰⁵ | 7 | 10/106 | 0.09 |
| II | 6 | 15/790,000 | 1.9 × 10⁻⁰⁵ | 6 | 28/84 | 0.33 |
| III | 3 | 12/274,000 | 4.4 × 10⁻⁰⁵ | 3 | 19/47 | 0.40 |
| IV | 2 | 0/270,000 | 0 | 2 | 11/39 | 0.28 |
| Total¹ | 17 | 28/1,858,000 | 1.5 × 10⁻⁰⁵ | 18 | 68/276 | 0.25 |

⁽¹⁾Cumulated throughout the tested serums.
⁽²⁾Positive clones divided by the whole number of clones tested.

Primary screening of a testis cDNA library was carried out successively with 17 individual serums originating from patients suffering from tumors in the indicated stage. The number of positive and total plaques assayed are shown in the 3^{rd} column. During the secondary screening every positive plaque (28) was subsequently tested with up to 18 individual serums from different patients in the indicated tumor stage.

The number and probability of all serological responses are comprised with respect to the tumor stage of the patients from which serums were withdrawn for screening a testis cDNA library. Although the data show a greater probability as to the presence of antibodies against tumor antigens (with the peak in stage III), these differences are not statistically relevant.

TABLE 3
Serological analyses of the identified clones

| | |
|---|---|
| CTCL | Controls |

| clone | reactive | n | reactive | n |
|---|---|---|---|---|
| se1-1 | 50% | 10 | 0% | 5 |
| se2-1 | 22% | 18 | 0% | 10 |
| se2-2 | 33% | 9 | 0% | 8 |
| se2-5 | 30% | 10 | 0% | 5 |
| se14-3 | 11% | 9 | 0% | 5 |
| se20-4 | 40% | 10 | 0% | 5 |
| se20-7 | 30% | 10 | 0% | 7 |
| se20-9 | 25% | 8 | 0% | 5 |
| se20-10 | 11% | 18 | 0% | 10 |
| se33-1 | 29% | 17 | 0% | 10 |
| se37-2 | 29% | 17 | 0% | 10 |
| se57-1 | 33% | 15 | 0% | 9 |
| se70-2 | 10% | 10 | 0% | 6 |
| se89-1 | 44% | 18 | 0% | 9 |
| L14-2 | 42% | 19 | 0% | 5 |
| L15-7 | 33% | 21 | 0% | 7 |
| Li9-1 | 19% | 21 | 0% | 5 |
| Li9-4 | 57% | 21 | 0% | 6 |
| Lg1-2 | 56% | 16 | 0% | 9 |
| Lii5-2 | 20% | 15 | 0% | 8 |
| Lii10-6 | 6% | 16 | 0% | 8 |
| Liii4-5 | 29% | 17 | 0% | 8 |

The table shows the percentage reactivity of the serums (number n) against the tested clones in the secondary screening.

The percentages of reacting serums and total number of tested serums (n) during secondary screening are indicated. Clones se2-1 and se20-4 each show additionally one of the homologous clones since they differed as to their reaction pattern, presumably because of sequence differences.

Five antigens represent formerly unknown sequences (se2-5, se20-10, se57-1, se70-2 and Lg1-2). The RNA expression pattern, analyzed by means of RT-PCR, of the identified antigens varied between highly restricted and ubiquitous expression in 28 normal, 17 CTCL tumor tissues and 33 tumor cell lines of different origins (Tables 4 and 5).

TABLE 4
Expression analysis by RT-PCR with antigen-specific
primers and cDNA from different tissues and cell lines

| cDNA | controls | | | | | |
|---|---|---|---|---|---|---|
| Primer | multi-tissue | activated | tumor tissue | cell lines | | |
| against | panels⁽¹⁾ | CTLs⁽²⁾ | CTCL | CTCL | leukemia | melanoma |
| clone | (n) | (n = 3) | (n) | (n = 4) | (n = 5-6) | (n) |
| se1-1 | 100% (17) | 100% | 91% (11) | 100% | 100% | 100% (6) |
| se2-1 | 4% (28)^{(1,3)} | 0% | 6% (17) | 0% | 0% | 0% (11) |
| se2-2 | 100% (18) | 100% | 90% (10) | 100% | 83% | 80% (5) |
| se2-5 | 94% (18) | 0% | 55% (11) | 0% | 100% | 100% (11) |
| se14-3 | 100% (18) | 100% | 100% (11) | 100% | 100% | 100% (11) |
| se20-4 | 100% (18) | 100% | 92% (12) | 100% | 100% | 100% (11) |
| se20-7 | 100% (15) | 100% | 100% (11) | 100% | 50% | 80% (5) |
| se20-9 | 100% (18) | 100% | 82% (11) | 100% | 83% | 45% (11) |
| se20-10 | 46% (28)⁽³⁾ | 67% | 77% (13) | 100% | 100% | 55% (11) |
| se33-1 | 61% (28)⁽³⁾ | 100% | 75% (16) | 100% | 83% | 100% (11) |
| se37-2 | 100% (16) | 100% | 93% (15) | 100% | 83% | 75% (5) |
| se57-1 | 21% (28)⁽³⁾ | 0% | 6% (17) | 0% | 0% | 0% (23) |
| se70-2 | 54% (28)⁽³⁾ | 33% | 31% (16) | 100% | 100% | 45% (22) |
| se89-1 | 87% (23) | 100% | 75% (16) | 100% | 100% | 73% (22) |
| L14-2 | 75% (24) | 33% | 40% (15) | 50% | 60% | 10% (21) |
| L15-7 | 65% (26) | 100% | 33% (15) | 50% | 40% | 20% (20) |
| Li9-1 | 85% (26) | 33% | 80% (15) | 75% | 100% | 42% (12) |
| Li9-4 | 92% (26) | 100% | 73% (15) | 75% | 80% | 60% (15) |
| GBP-TA | 32% (28)^{(3,4)} | 0% | 26% (19) | 75% | 20% | 0% (23) |
| Lii5-2 | 59% (22) | 0% | 21% (14) | 25% | 75% | 21% (24) |
| Lii10-6 | 86% (21) | 100% | 100% (14) | 50% | 100% | nt |
| Liii4-5 | 55% (22) | 33% | 29% (14) | 100% | 75% | nt |

The table shows the percentage frequency of the expression in different tissues and cell lines according to RT-PCR analysis (number of tissues is given in parentheses; nt: not tested).
⁽¹⁾RT-PCRs with testis cDNA always yielded postivie results. In the case of clone se2-1 testis cDNA was the only positive sample. Composition of control panel see Table 5.
⁽²⁾Activated cytotoxic T-cells.
⁽³⁾Details on these results see Table 5.
⁽⁴⁾GBP-TAshort with 7%.

TABLE 5
RT-PCR analyses by specific primers against differentially
expressed sequences and multiple tissue (MTC) cDNA

| | Primer against clone: | | | | | |
|---|---|---|---|---|---|---|
| | | | | | | GBP-TA/- |
| | se2-1 | se20-10 | se33-1 | se57-1 | se70-2 | TAshort |
| intestines | − | − | − | + | − | − |
| small intestine | − | + | + | + | + | −/+ |
| fetal liver | − | − | − | − | + | −/+ |
| fetal lung | − | − | + | − | + | − |
| fetal spleen | − | − | + | − | + | −/+ |
| fetal kidney | − | + | + | − | + | − |
| fetal skeletal | − | − | + | − | + | − |
| muscle | | | | | | |
| fetal thymus | − | − | − | − | + | −/+ |
| fetal brain | − | − | + | − | + | − |
| fetal heart | − | − | + | − | + | −/+ |
| brain | − | + | + | − | + | − |
| skin | − | − | + | − | − | − |
| heart | − | + | − | − | − | − |
| bone marrow | − | + | + | + | − | + |
| liver | − | − | + | − | − | − |
| lung | − | − | + | − | − | − |
| stomach | − | + | + | − | − | + |
| spleen | − | + | + | + | + | −/+ |
| kidney | − | + | + | − | + | − |
| ovary | − | − | + | − | − | − |
| pancreas | − | − | + | − | − | − |
| periph blood | − | − | − | − | − | − |
| lymphocytes | | | | | | |
| placenta | − | + | + | − | − | −/+ |
| prostate | − | + | + | − | − | − |
| skeletal muscle | − | − | − | − | − | − |
| testis | + | + | + | + | + | − |
| thymus | − | − | + | − | + | − |
| trachea | − | + | + | + | + | − |

Either commercially available cDNA assortments were used or cDNA was prepared from RNA assortments (Clontech). Each sample contained cDNA from samples from several individuals. The skin cDNA was prepared from a single sample. RT-PCR against GBP-TA/GB-Tashort distinguishes between both variants.

### EXAMPLE 3

### Tumor-Specific Antigens

Six clones (represented by at least four different recombinants) were homologous to SCP-1, a protein connected with meiosis (Türeci et al. , PNAS U.S.A. 95 (1998) , 5211-5216) . Interestingly enough, the serological reactivity of different serums differed between the different Scp-1 clones: clone se2-1 was determined by serums from 2/15 MF patients and 2/3 patients suffering from Sézary syndrome. By means of RT-PCR it was proved that se2-l is tumor-specific. Another clone homologous to SCP-1 (se37-1) was identified by 3/9 MF serums and 1/3 Sézary syndrome serums. This might reflect different epitopes of SCP-1, since the clones differed as regards length. Clone se33-2 also reacted with 1/5 control serums. Interestingly enough, this clone encoded another peptide sequence within the first reading frame, which was not present in the other clones homologous to SCP-1 and thus could represent an autoantigen responsible for the reactivity of the control serum. The PCR analyses were carried out using the same primers as published by Tureci et al. (1998), which also perfectly fits the clones homologous to SCP-1. Of all the tested normal tissues and tumor samples and cell lines only one testis sample and one MF sample (patient H.S.) was found which expressed SCP-1 mRNA. The positive result of the MF-cDNA could be confirmed by Northern blot yielding a band of about 4.3 kb. Furthermore, the serum of the patient H.S. also reacted with se2-1 and one of the other clones homologous to SCP-1. In addition, according to Northern blot analysis clones se57-1 and L15-7 are tumor-specific.

### EXAMPLE 4

### (A) Antigens with Limited Expression Pattern and (B) Ubiquitously Expressed Antigens

13 antigens with differential or ubiquitous expression (detected by means of RT-PCR) are described below (see Tables 4 and 5).

### (A)

For five new antigens (se2-5, se20-10, se57-1, se70-2 and Lg1-2) and two antigens with homologies to known genes (se33-1: NP220; se89-1: RAP140 protein correlated with retinoblastoma) a differential expression showed on a molecular level. The serological reactivity to these clones (defined as percentage of reactive serums during secondary screening) was 31% on the average. A minor reactivity rate showed with respect to clones se20-10 and se70-2 (2/18 and 1/18 reactive serums, respectively), whereas 71% of the serums from CTCL patients (n=14) reacted positively with clone se89-1. All of the 6 clones with up to 10 control serums showed no reaction.

It was started quantifying the RT-PCR results by means of Northern analyses. Such antigens which in normal tissues do not prove to be equally expressed as compared to tumor samples are considered to be potential therapeutic agents.

It was possible to show by means of RT-PCR analyses that the se-2-5-specific m-RNA is expressed almost ubiquitously in normal tissues but not in activated T-cells and only in 55% of the CTCL tissue samples. In contrast therewith, the Northern blot analysis showed a limited expression pattern even within normal tissues. Intense signals were detectable in kidney, esophagus and testis, weaker ones were identifiable in colon, small intestine, thymus, bone marrow and stomach. While in all of the positive normal tissues three bands were detectable (5.2, 5.4 and 3.9 kB), the only positive CTCL cell line SeAx showed a signal at 3.9 kb. Specific mRNAs for clones se20-10 and se57-1 were found by means of RT-PCR in 43% of the studied control tissues and 21% thereof, respectively. Interestingly enough, the expression of the mRNA specific to se57-1 was greatly down-regulated in all of the tumor tissues and cell lines. In contrast therewith, the expression of the mRNA of clone se70-2 was up-regulated (100%) as compared to normal tissues (54%) in the CTCL and leukemia cell lines, whereas the CTCL tissues and melanoma cell lines showed mean expression levels (33% and 45%, respectively) . All the fetal tissues of the control tissues were RT-PCR positive.

A differential expression showed for two antigens with homologies to known sequences: se33-1 cDNA is homologous to NP220, a DNA-binding protein, and se89-1 cDNA is homologous to RAP140, a retinoblastoma-associated clone. Clone se33-1 showed 99% similarity at its 3′ end of NP220 over an overlapping distance of 3830 bp, however this clone is shortened at its 5′ end, which results in a shortened ORF. The RT-PCR (using se33-1-specific primers) furnished proof of mRNA in 6/8 fetal and 16/20 normal tissues (Table 5) and in CTCL tissues (12/16), activated cytotoxic T-cells and in most cell lines (Table 4).

cDNA of clone se89-1 showed 99% similarity with respect to RAP140 in an overlapping region of 3444 bp and a gap of 60 bp which is located within the ORF and results in an amino acid gap of 20 amino acids. RT-PCR was carried out using different primers: first with the primer combination RAP140 (see Table 1 and Example 1), both RAP140 and se89-1 being identified and three bands showing in testis-cDNA and two bands turning up in various other cDNAs. For the specification of se89-1 expression a new reverse primer (see Table 1) was designed which spans the gap in se89-1. Using this primer together with the forward primer against RAP140, which also detects se89-1, only one band was amplified. The frequency of the positive cDNAs with respect to the se89-1-specific primers did not distinguish substantially between the control tissues (79%), CTCL tissues (75%) and cell lines (CTCL and leukemia cell lines: 100%, melanoma lines: 73%). By means of Northern blot analyses it was possible to confirm the presence of mRNA specific to se89-1 within mRNA originating from brain, kidney, colon and testis and the CTCL line SeAx.

### (B)

Six antigens homologous to known sequences (se1-1, se2-2, se14-3, se20-4, se20-9 and se37-2) proved to be expressed ubiquitously. In all of the control tissues (n=28) mRNA specific to these clones could be detected by means of RT-PCR. In the two clones se14-3 and se20-4, all of the tumor tissues and cell lines were also positive in the RT-PCR. In contrast therewith, clones se2-2, se20-7, se20-9 and se37-2 were only expressed in a subgroup of the melanoma and leukemia cell lines while CTCL tissue and CTCL cell lines showed a greater percentage of cDNAs positive in the RT-PCR.

The reactivity of patient serums with these clones was about 29% on the average, two extremes also being observable: The reactivity to clone se14-3 was revealed in 11% (1/9) and that to clone se1-1 in 50% (5/10) of the CTCL serums. Two control serums (n=10) reacted with clone se20-6 which is homologous to clone se20-4. In connection with se20-6 it was possible to show that the latter codes for a different peptide (72 aa) in the first reading frame, which was not present in either se20-4 or its homologous gene HRIHFB2216. The sequence analysis of these clones and a comparison with the homologous counterparts disclosed in some cases insertions, deletions or elongations.

It should be pointed out that all of the tested clones are serologically specific.

### EXAMPLE 6

### Sequence Analyses as Regards Lg1-2

A number of further clones having major correspondence with Lg1-2 could be isolated, which were completed in the 3′ direction (stop codon and 3′-untranslated region is available). This is shown in FIG. 24. These clones could be comprised in a gene (GBP-TA) which was assignable to the 1p22.3 chromosome. In this connection, it was possible to differentiate between two splicing variants: GBP-TA were assigned to 12 exons while exon no. 2 lacked from GBP-TAₛₕₒᵣₜ.

It was possible to derive from GBP-TA a protein which has certain homologies to the known guanylate-binding proteins GBP-1, GBP-2 and HGBP (U.S. Pat. No. 5,871,965). However, the sequence of HGBP does not comprise exon 2.

### EXAMPLE 7

### Expression Analyses with Respect to GBP-TA

RT-PCR experiments were carried out as to the analysis of GBP-TA expression. Two different primer pairs (see FIG. 24) were used for differentiating the two splicing variants. Here, a large number of control cDNAs were used, each prepared from a collection of tissues from different donors. While 11 control tissues were negative for both primers, GBP-TAₛₕₒᵣₜ was detected in 5 tissues and both variants of GBP-TA were only found in 2 tissues (bone marrow and stomach) (Table 6).

TABLE 6
Detection of GBP-TA and GBP-TAₛₕₒᵣₜ in adult control
tissues

result

| Control tissue | Primer set I | primer set II |
|---|---|---|
| Brain, colon, heart, | − | − |
| kidney, liver, ovary, | | |
| lung, PMNC, prostate, | | |
| testis, thymus, | | |
| trachea | | |
| placenta, small intestine | − | + |
| spleen, activ. CD8 | | |
| T-cells, uterus | | |
| Bone marrow, stomach | + | + |

In contrast to the control tissues, GBP-TA was detected in various tumor tissues by means of RT-PCR: cutaneous T-cell lymphomas (26%, n=19), tumors from the HNO region (21%, n=14) . GBP-TAshort was found in 20% of the HNO tumors (n=15) and 9% of the colon carcinomas (n=35) . Se57-1 was detected in 20% of the colon carcinomas (n=35) and 57% of the HNO tumors (n=28).

Since RT-PCR is extremely sensitive and allows no statement on the presence and amount of protein, the expression was checked by means of Western blot and a GBP-TA-specific antibody. Of the RT-PCR-positive controls several could be tested as protein medleys in a Western blot: placenta, small intestine, spleen, fetal liver, stomach, testis, uterus. Like other control protein medleys tested (mammary gland, testis) they also proved to be negative, while proteins obtained from tumors (CTCL) cell lines (SeAx, MyLa, Hut-78, HH; isolation via Tristar, AGS, Heidelberg, Germany) showed a marked band of corresponding size. This proves that GBP-TA is suited as a specific target structure for the therapy.

### EXAMPLE 8

### Preparation of Antibodies Against GBP-TA

For the preparation of a GBP-TA-specific antibody, the insert of a clone comprising GBP-TA bases from 539 to 1991 inclusive, was cloned into a His vector and expressed in E. coli. The recombinant protein was purified on a nickel column, then separated in an SDS gel for further purification and the corresponding band was cut out. A rabbit was immunized with the cut-out gel piece, whose preimmune serum did not react with the antigen.

Immunization Protocol for Polyclonal Antibodies in Rabbits

600 μg of purified KLH-coupled peptide in 0.7 ml PBS and 0.7 ml complete or incomplete Freund's adjuvant are used per immunization:

Day 0: 1^{st} immunization (complete Freund's adjuvant)

Day 14:2^{nd} immunization (incomplete Freund's adjuvant; icFA)

Day 28: 3^{rd} immunization (icFA)

Day 56: 4^{th} immunization (icFA)

Day 80: bleeding to death.

The rabbit serum is tested in an immunoblot. For this purpose, a peptide used for the purpose of immunization is subjected to SDS polyacrylamide gel electrophoresis and transferred to a nitrocellulose filter (cf. Khyse-Andersen, J., J. Biochem. Biophys. Meth. 10 (1984), 203-209). The Western blot analysis was carried out as described in Bock, C.-T. et al., Virus Genes 8, (1994), 215-229. For this purpose, the nitrocellulose filter is incubated with a first antibody at 37° C. for one hour. This antibody is the rabbit serum (1:10000 in PBS). After several wash steps using PBS, the nitrocellulose filter is incubated with a second antibody. This antibody is an alkaline phosphatase-coupled monoclonal goat anti-rabbit IgG antibody (Dianova company) (1:5000) in PBS. 30 minutes of incubation at 37° C. are followed by several wash steps using PBS and subsequently by the alkaline phosphatase detection reaction with developer solution (36 μM 5′-bromo-4-chloro-3-indolylphosphate, 400 μM nitroblue tetrazolium, 100 mM Tris-HCl, pH 9.5, 100 mM NaCl, 5 mM MgCl₂) at room temperature until bands become visible.

It shows that polyclonal antibodies according to the invention can be prepared.

### EXAMPLE 9

### ELISA

A GBP-TA insert the same as that used for the antibody collection (bases no. 539 to 1991 inclusive of GBP-TA) was cloned into a pGEX vector, expressed recombinantly and used in a GST-ELISA. The ELISA system used follows Sehr et al. (J. of Immunol. Meth. 2001, 253, 153-162). For this purpose, glutathione-casein-coated ELISA plates were loaded with the fusion protein and then serums from CTCL patients and control persons were tested for the presence of specific antibodies. It turned out that 17% of the CTCL serums (n=60) and only 2% of the control serums (n=99) reacted with the GST-GBP-TA fusion protein.

The ELISA is suited for all the indicated marker antigens for diagnostic purposes, for a prognostic estimation and for a follow-up.

1. A diagnostic composition comprising at least one nucleic acid sequence whose modified expression is associated with a tumoral disease, wherein the nucleic acid sequence comprises Lg1-2 (FIG. 5) or GPB-TA (FIG. 23).

2. A medicament comprising at least one nucleic acid sequence, whose modified expression is associated with a tumoral disease, wherein the nucleic acid sequence comprises Lg1-2 (FIG. 5) or GBP-TA (FIG. 23).

3. The diagnostic composition according to claim 1, wherein the nucleic acid sequence whose modified expression is connected with a tumoral disease, comprises a nucleic acid sequence:
(a) which on account of the degeneration of the genetic code differs from a nucleic acid sequence defined in claim 1 as regards the codon sequence;
(b) which hybridizes with a nucleic acid sequence of claim 1; or
(c) which is a fragment, an allelic variant or another variant of a nucleic acid sequence of claim 1.

4. The nucleic acid sequence of claim 1 is a cDNA or a genomic DNA.

5. A protein whose modified concentration is connected with a tumoral disease and is encoded by a nucleic acid sequence of claim 1.

6. A diagnostic composition comprising at least one vector, wherein the vector comprising one of the nucleic acid sequences according to claim 1, at least one protein encoded by a nucleic acid sequence according to claim 1, or at least one antibody directed against the protein.

7. The diagnostic composition according to claim 6, wherein the composition is used for the diagnosis or follow-up of a tumoral disease.

8. The diagnostic composition according to claim 7, wherein the composition is provided in the form of an ELISA, protein chip, nucleic acid chip or a membrane loaded with DNA, RNA or protein.

9. A medicament comprising at least one vector, wherein the vector comprising one of the nucleic acid sequences according to claim 2, at least one protein encoded by a nucleic acid sequence according to claim 2, or at least one antibody directed against the protein.

10. The medicament according to claim 9, wherein the medicament is used for treating tumoral diseases.

11. A method of using a nucleic acid sequence according to claim 1, wherein the nucleic acid is used for diagnosis or therapy of a tumoral disease.

12. A method of using a protein encoded by a nucleic acid sequence of claim 1, wherein the protein is used for diagnosis or therapy of a tumoral disease.

13. A method of using an antibody directed against a protein encoded by a nucleic acid sequence of claim 1, wherein the antibody is used for diagnosis or therapy of a tumoral disease.

14. A method of using a protein encoded by a nucleic acid of claim 1 or an antibody directed against the protein, wherein the antibody or the protein is used as a vaccination agent.

15. The method of claim 12, wherein the protein is used for preparation of peptide-loaded antigen-presenting cells.

16. The method of claim 12, wherein the protein is used for preparation of tumor-specific T-cells.

17. The diagnostic composition according to claim 1, wherein the tumoral disease is cutaneous T-cell lymphomas.

18. The medicament according to claim 2, wherein the tumoral disease is cutaneous T-cell lymphomas.

19. The method according to claim 11, wherein the tumoral disease is cutaneous T-cell lymphomas.
